(19) United States
(12) Patent Application Publication
(10) Pub. No.: US 2025/0304689 A1
(43) Pub. Date: Oct. 2, 2025

(54) BISPECIFIC ANTIBODIES AGAINST CD3 AND CD20

(71) Applicant: GENMAB A/S, Valby (DK)

(72) Inventors: Brian ELLIOTT, Hoboken, NJ (US); Christopher W.L. CHIU, WARREN, NJ (US); Nurgul KILAVUZ, Chatham, NJ (US); Mariana SACCHI, Skillman, NJ (US)

(21) Appl. No.: 18/855,842

(22) PCT Filed: Apr. 13, 2023
(86) PCT No.: PCT/EP2023/059697

§ 371 Date: Oct. 10, 2024

Related U.S. Application Data
(60) Provisional application No. 63/330,535, filed on Apr. 13, 2022.
(60) Provisional application No. 63/330,662, filed on Apr. 13, 2022.
(60) Provisional application No. 63/421,707, filed on Nov. 2, 2022.
(60) Provisional application No. 63/430,875, filed on Dec. 7, 2022.

(51) Int. Cl. C07K 16/28; A61K 39/00; A61P 35/00

(52) U.S. Cl. CPC C07K 16/2809; A61P 35/00; C07K 16/2887; A61K 2039/505; A61K 2039/545; C07K 2317/31

(57) ABSTRACT

The present invention relates to bispecific antibodies (bsAbs) and the use of such antibodies in the treatment of disease in subjects. Moreover, advantageous treatment regimens are provided for the treatment of B-cell Non-Hodgkin Lymphoma (B-NHL).

### FIELD

The present invention relates to bispecific antibodies (bsAbs) targeting both CD3 and CD20 and the use of such antibodies in the treatment of disease in subjects. Moreover, advantageous treatment regimens are provided.

### INTRODUCTION

Monoclonal antibodies (mAbs) have been shown to be highly successful for the treatment of cancer. A further promising approach to improve antibody therapy is by recruiting T cells specifically to the antigen-expressing cancer cells. This can be achieved by utilizing bsAbs targeting both T cells and antigen-expressing cells. However, initial clinical studies were rather disappointing mainly due to low efficacy, severe adverse effects (cytokine storm) and immunogenicity of the bispecific antibodies. Advances in the design and application of bispecific antibodies have partially overcome the initial barrier of cytokine release syndrome and improved clinical effectiveness without dose-limiting toxicities (Garber, 2014, Nat. Rev. Drug Discov. 13:799-801).

The CD20 molecule, also called human B-lymphocyte-restricted differentiation antigen or Bp35, is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin lymphomas (B-NHL), but is not found on hematopoietic stem cells, pro-B cells, normal plasma cells, or other normal tissues. Methods for treating cancer as well as autoimmune and immune diseases by targeting CD20 are known in the art.

For example, the chimeric CD20 antibody rituximab has been used for or suggested for use in treating cancers such as B-NHL and chronic lymphocytic leukemia (CLL). The human monoclonal anti-CD20 antibody ofatumumab has been used for or suggested for use in treating among others various CLL indications, follicular lymphoma (FL), neuromyelitis optica (NMO), diffuse and relapsing-remitting multiple sclerosis (RRMS).

Currently, bispecific antibodies are under development that target both CD20 and CD3. For example, WO2011028952 describes amongst others the generation of CD3×CD20 bispecific molecules using Xencor's XmAb bispecific Fc domain technology, WO2014047231 describes REGN1979 and other CD3×CD20 bispecific antibodies generated using the FcAAdp technology from Regeneron Pharmaceuticals, and Sun et al. (2015, Science Translational Medicine 7, 287ra70) describe a B cell-targeting anti-CD20/CD3 T cell-dependent bispecific antibody constructed using “knobs-into-holes” technology. Such bispecific antibodies are currently being tested in clinical trials for specific indications in humans.

A bispecific antibody of particular interest that is under development is epcoritamab (Duobody CD3×CD20; GEN3013) (Engelberts et al., 2020, EBioMedicine, Vol. 52, 102625, WO2016110576, and WO2019155008, incorporated herein by reference).

Although there are currently treatment regimens available for the treatment of CD20+ cancers, such as B-NHL, there is still a need for further therapeutic options, as there still remain patients that relapse or are refractory to currently available treatments. Epcoritamab is a candidate that can add to the repertoire of treatments options that may benefit patients suffering from cancers such as B-NHL.

Hence, one object of the present invention is to provide for means and methods for treating a cancer by using a bispecific antibody targeting CD3×CD20 as described herein, such as epcoritamab, as well as providing a bispecific antibody targeting CD3×CD20 as described herein, such as epcoritamab, for use in the treatment of a cancer.

Specific dose ranges and/or dosage regimens are provided that are advantageous with regard to such methods or uses, such as for a cancer known or identified as being positive for CD20, such as a B-NHL. More in particular, specific dose ranges and/or dosage regimens are provided herein that are advantageous with regard to the treatment of patients suffering from diffuse large B-cell lymphoma (DLBCL), High Grade B-cell lymphoma, FL. The dose ranges and/or dosage regimens provided herein were assessed to be safe for human use and/or shown to be highly effective in the treatment of B-NHL.

### FIGURES

FIG. 1. Overview of trial design, GCT3013-01

FIG. 2: Expansion Scheme, GCT3013-01.

FIG. 3: Waterfall plot of best reduction in sum of the products of diameters based on IRC assessment according to Lugano criteria in the expansion aNHL cohort.

FIG. 4: Overall Survival by Best Overall Response, GCT3013-01 expansion.

FIG. 5: Probability of progression free survival (PFS) across MRD positive patients and subgroups of MRD negative patients.

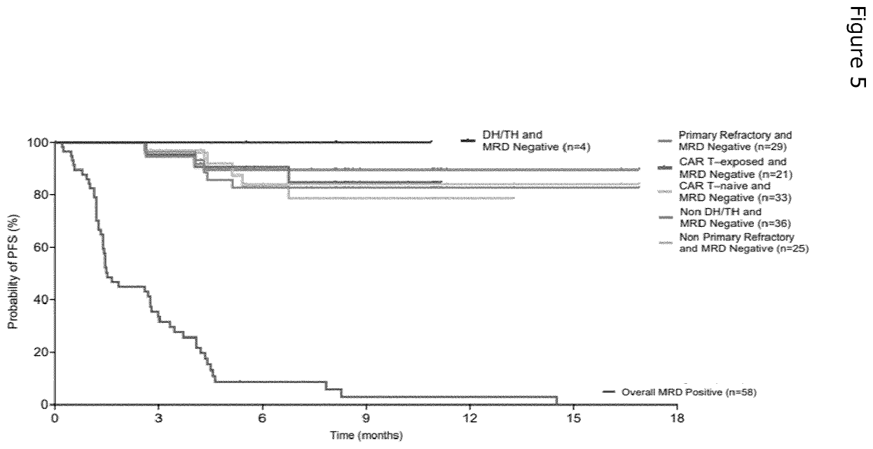

### DETAILED DESCRIPTION

The term “immunoglobulin” herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH or VH) and a heavy chain constant region (abbreviated herein as CH or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules (Brochet X., Nucl Acids Res. 2008; 36: W503-508 and Lefranc MP., Nucleic Acids Research 1999; 27:209-212; see also internet http address http://www.imgt.org/). Unless otherwise stated or contradicted by context, reference to amino acid positions in the constant regions in the present invention is according to the EU-numbering (Edelman et al., Proc Natl Acad Sci USA. 1969 May; 63 (1): 78-85; Kabat et al., Sequences of Proteins of Immunological Interest, Fifth Edition. 1991 NIH Publication No. 91-3242). For example, SEQ ID NO: 15 herein sets forth amino acid positions 118-447 according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antibody-binding region”, as used herein, refers to the region which interacts with the antigen and comprises both the VH and the VL regions. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21 (11): 484-90); (vi) camelid or nanobodies (Revets et al; Expert Opin Biol Ther. 2005 January; 5 (1): 111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “bispecific antibody” or “bs” or “bsAb” in the context of the present invention refers to an antibody having two different antigen-binding regions defined by different antibody sequences. A bispecific antibody can be of any format.

When used herein, the terms “half molecule”, “Fab-arm” and “arm” refer to one heavy chain-light chain pair.

When a bispecific antibody is described to comprise a half-molecule antibody “derived from” a first parental antibody, and a half-molecule antibody “derived from” a second parental antibody, the term “derived from” indicates that the bispecific antibody was generated by recombining, by any known method, said half-molecules from each of said first and second parental antibodies into the resulting bispecific antibody. In this context, “recombining” is not intended to be limited by any particular method of recombining and thus includes all of the methods for producing bispecific antibodies described herein below, including for example recombining by half-molecule exchange (also known as “controlled Fab-arm exchange”), as well as recombining at nucleic acid level and/or through co-expression of two half-molecules in the same cells.

The term “full-length” when used in the context of an antibody indicates that the antibody is not a fragment but contains all of the domains of the particular isotype normally found for that isotype in nature, e.g. the VH, CH1, CH2, CH3, hinge, VL and CL domains for an IgG1 antibody. A full-length antibody may be engineered. An example of a “full-length” antibody is epcoritamab.

When used herein, unless contradicted by context, the term “Fc region” refers to an antibody region consisting of the Fc sequences of the two heavy chains of an immunoglobulin, wherein said Fc sequences comprise at least a hinge region, a CH2 domain, and a CH3 domain.

When used herein the term “heterodimeric interaction between the first and second CH3 regions” refers to the interaction between the first CH3 region and the second CH3 region in a first-CH3/second-CH3 heterodimeric protein.

When used herein the term “homodimeric interactions of the first and second CH3 regions” refers to the interaction between a first CH3 region and another first CH3 region in a first-CH3/first-CH3 homodimeric protein and the interaction between a second CH3 region and another second CH3 region in a second-CH3/second-CH3 homodimeric protein.

As used herein, the term “binding” in the context of the binding of an antibody to a predetermined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

In a preferred embodiment, the antibody of the invention is isolated. An “isolated antibody” as used herein, is intended to refer to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein, refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95LI7), a CD3δ (delta) chain (human CD3δ UniProtKB/Swiss-Prot No P04234, or cynomolgus monkey CD3δ UniProtKB/Swiss-Prot No Q95LI8), two CD3ε (epsilon) chains (human CD3ε UniProtKB/Swiss-Prot No P07766 (SEQ ID NO: 28); cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ε UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

A “CD3 antibody” or “anti-CD3 antibody” is an antibody which binds specifically to the antigen CD3, in particular human CD38 (epsilon) (SEQ ID NO: 28).

The term “human CD20” or “CD20” refers to human CD20 (UniProtKB/Swiss-Prot No P11836) (SEQ ID NO: 29) and includes any variants, isoforms and species homologs of CD20 which are naturally expressed by cells, including tumor cells, or are expressed on cells transfected with the CD20 gene or cDNA. Species homologs include rhesus monkey CD20 (Macaca mulatta; UniProtKB/Swiss-Prot No H9YXP1) and cynomolgus monkey CD20 (Macaca fascicularis; UniProtKB No G7PQ03).

A “CD20 antibody” or “anti-CD20 antibody” is an antibody which binds specifically to the antigen CD20, in particular to human CD20 (SEQ ID NO: 29).

A “CD3×CD20 antibody”, “anti-CD3×CD20 antibody”, “CD20×CD3 antibody” or “anti-CD20×CD3 antibody” is a bispecific antibody, which comprises two different antigen-binding regions, one of which binds specifically to the antigen CD20 and one of which binds specifically to CD3.

“DuoBody-CD3×CD20” herein refers to an IgG1 bispecific CD3×CD20 antibody comprising a first heavy and light chain pair as defined in SEQ ID NO: 24 and SEQ ID NO: 25, respectively, and comprising a second heavy and light chain pair as defined in SEQ ID NO: 26 and SEQ ID NO: 27. The first heavy and light chain pair comprises a binding region binding to human CD3ε (epsilon), the second heavy and light chain pair comprises a binding region binding to human CD20. The first binding region comprising the VH and VL sequences as defined by SEQ ID NO. 6 and 7, and the second binding region comprising the VH and VL sequences as defined by SEQ ID NO.13 and 14. This bispecific antibody can be prepared as described in WO 2016/110576.

The present invention also provides antibodies comprising functional variants of the heavy chain, light chains, VL regions, VH regions, or one or more CDRs of the antibodies of the examples. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity for particular epitopes of CD20 and/or CD3, Fc inertness and PK parameters such as half-life, Tmax, Cmax.

Such functional variants typically retain significant sequence identity to the parent antibody and/or have substantially similar length of heavy and light chains. The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

Exemplary variants include those which differ from heavy and/or light chains, VH and/or VL and/or CDR regions of the parent antibody sequences mainly by conservative substitutions; for instance, 10, such as 9, 8, 7, 6, 5, 4, 3, 2 or 1 of the substitutions in the variant may be conservative amino acid residue replacements.

In the context of the present invention, conservative substitutions may be defined by substitutions within the classes of amino acids reflected in the following table:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

In the context of the present invention the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

The term “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen binding peptide (in other words, the amino acid residue is within the footprint of the specifically antigen binding peptide).

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. The VH and VL of the CD3 arm that is used herein in Duobody-CD3×CD20 represents a humanized antigen-binding region. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The VH and VL of the CD20 arm that is used herein in Duobody-CD3×CD20 represents a human antigen-binding region. Human monoclonal antibodies of the invention can be produced by a variety of techniques, including conventional monoclonal antibody methodology, e.g., the standard somatic cell hybridization technique of Kohler and Milstein, Nature 256:495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery, see e.g. Mendez et al. 1997 Nat Genet. 15 (2): 146-56. The VH and VL of the CD20 arm that is used herein in Duobody-CD3×CD20 represents a human antigen-binding region.

The term “reducing conditions” or “reducing environment” refers to a condition or an environment in which a substrate, here a cysteine residue in the hinge region of an antibody, is more likely to become reduced than oxidized.

The term “recombinant host cell” (or simply “host cell”), as used herein, is intended to refer to a cell into which an expression vector has been introduced, e.g. an expression vector encoding an antibody of the invention. Recombinant host cells include, for example, transfectomas, such as CHO, CHO-S, HEK, HEK293, HEK-293F, Expi293F, PER.C6 or NS0 cells, and lymphocytic cells.

The term “treatment” refers to the administration of an effective amount of a therapeutically active antibody of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

The term “Double/triple hit lymphoma” (DH/TH) refers to an aggressive diffuse large B cell lymphoma (DLBCL), defined as having concurrent MYC, BCL2, and/or BCL6 gene rearrangements. DH/TH is also known as high-grade B-cell lymphoma (HGBL).

The term “non-DH/TH” refers to DLBCL patients, who do not qualify as DH/TH, based on above gene rearrangements.

The term “non-DLBCL subtypes” includes HGBL with MYC and BCL2 and/or BCL6 rearrangements, HGBCL NOS, PMBCL, and FL grade 3B (as per foot note to the table)

The term “Primary refractory” refers to a subject or disease, in particular large B cell lymphoma (LBCL), which has progressed within 6 months following first line therapy.

The term “CAR T-naïve” refers to a subject who has not received prior therapy with chimeric antigen T cell receptor (CAR T) therapy.

The term “CAR T-exposed” refers to a subject who has received prior therapy with CAR T therapy.

The term “refractory to prior CAR T” refers to a subject or disease, in particular large B cell lymphoma (LBCL), which has progressed within 6 months following prior CAR T therapy.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/v or less, such as 3% w/v or less, such as 1% w/v or less such as 0.4% w/v or less, such as below 0.1% w/v or less, such as 0.04% w/v). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates such as polysorbate or polysorbate 80, propylene glycol dilaurate, propylene glycol monolaurate, sorbitan esters sucrose palmitate, sucrose stearate, tricaprylin and TPGS (Nonionic and zwitterionic surfactants).

A “diluent” of interest herein is one which is pharmaceutically acceptable (safe and non-toxic for administration to a human) and is useful for the preparation of dilutions of the pharmaceutical composition. Preferably such dilutions of the composition of the invention dilute only the antibody concentration but not the buffer and stabilizer. Accordingly, in a preferred embodiment the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution, Ringer's solution or dextrose solution. The diluent may comprise or consist essentially of acetate buffer, sorbitol and polysorbate 80 (e.g. 0.04% w/v). In a preferred embodiment the diluent comprises or consists essentially of 0.9% NaCl.

The terms “pharmaceutical composition” and “pharmaceutical formulation” is used interchangeably herein.

The term “systemic administration” refers to a route of administration of medication, nutrition or other substance into the circulatory system so that the entire body is affected. Administration can take place via enteral administration (absorption of the drug through the gastrointestinal tract) or parenteral administration (generally injection, infusion, or implantation). The term “systemic” when used in connection with a medication, nutrition or other substance refers to such medication, nutrition or other substance which is provided to a subject by systemic administration or is intended for systemic administration.

The term “minimal residual disease (MRD)” refers to the small number of cancer cells that remain in the body after treatment. The number of remaining cells may be sufficiently low to not cause any physical signs or symptoms and often cannot even be detected through traditional methods, such as viewing cells under a microscope and/or by tracking abnormal serum proteins in the blood. An MRD positive test result means that residual (remaining) disease was detected. A negative result means that residual disease was not detected.

### EMBODIMENTS

In one aspect, the current invention provides for a method of treating a B-cell non-Hodgkin lymphoma (B-NHL) in a human subject, the method comprising administering subcutaneously (sc) to a subject in need thereof, a bispecific antibody comprising
(i) a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
(ii) a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered at a dose of at least 24 mg and is administered to provide a response in said subject, the response having a duration of 6 months or longer.

In another aspect, the current invention provides for a method of treating a B-cell non-Hodgkin lymphoma (B-NHL) in a human subject, the method comprising administering subcutaneously to a subject in need thereof, a bispecific antibody, which is a full-length antibody with an inert Fc region, comprising
(i) a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
(ii) a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered at a dose of at least 24 mg and is administered to provide a response in said subject, the response having a duration of 6 months or longer.

In yet another aspect, the current invention provides for a method of treating a B-cell non-Hodgkin lymphoma (B-NHL) in a human subject, the method comprising administering subcutaneously to a subject in need thereof, a bispecific antibody, which is a full-length antibody, comprising
(i) a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
(ii) a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered at a dose of at least 24 mg and is administered to provide a response in said subject, the response having a duration of 6 months or longer.

In another aspect, the current invention provides for a method of treating a B-cell non-Hodgkin lymphoma (B-NHL) in a human subject, the method comprising administering subcutaneously to a subject in need thereof, a bispecific antibody, which comprises an inert Fc region, comprising
(i) a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
(ii) a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered at a dose of at least 24 mg and is administered to provide a response in said subject, the response having a duration of 6 months or longer.

As confirmed in the working examples of the present application, the response may have a duration considerably longer than 6 months, such as a duration of 7 months or longer, 8 months or longer, 9 months or longer, 10 months or longer, 11 months or longer, or such as a duration of 12 months or longer.

The bispecific antibody may be administered to the subject over a period which is 6 months or longer, such as 7 months or longer, such as 8 months or longer, 9 months or longer, 10 months or longer, 11 months or longer, or such as a duration of 12 months or longer.

In some embodiments, the response comprises at least a partial response (PR). Preferably the response comprises a complete response.

In further embodiments, the response comprises a complete response, and the response has a duration of 8 months or longer, such as a duration of 9 months or longer, 10 months or longer, 11 months or longer, 12 months or longer, 13 months or longer, 14 months or longer, 15 months or longer, 16 months or longer, 17 months or longer, 18 months or longer, 19 months or longer or such as 20 months or longer.

In some embodiments, the subject shows a best overall response (BOR) of partial response (PR).

In other embodiments, the subject shows a best overall response (BOR) of complete response.

In some embodiments, the subject shows a complete response, and the response has a duration of 8 months or longer, such as a duration of 9 months or longer, 10 months or longer, 11 months or longer, 12 months or longer, 13 months or longer, 14 months or longer, 15 months or longer, 16 months or longer, 17 months or longer, 18 months or longer, 19 months or longer or such as 20 months or longer.

In a further aspect, the invention provides a method of achieving negative minimal residual disease (MRD) status/MRD negativity in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO:6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered to said subject for a time and/or a number of treatment cycles sufficient to provide a negative minimal residual disease (MRD) status/MRD negativity.

Minimal residual disease (MRD) is emerging as a prognostic and sensitive assessment to measure depth of response, with studies demonstrating that undetectable MRD (also referred to as MRD negativity) is associated with improved PFS and OS in multiple myeloma. Moreover, MRD is becoming a key end point in recent clinical studies, and the International Myeloma Working Group (IMWG) has provided updated guidance for the uniform assessment and reporting of MRD negativity. The IMWG MRD criteria state that MRD should be assessed when a patient achieves complete response (CR) or better (≥CR), with a minimum sensitivity of 1 nucleated tumor cell in 100 000 normal cells (10⁻⁵ sensitivity threshold), by either next-generation sequencing (NGS) or next-generation flow cytometry.

In the methods according to the invention, providing treatment of lymphoma, circulating tumor DNA (ctDNA) levels may be assessed using tumor or peripheral blood is to identify tumor associated DNA sequences (rearranged B cell receptor genes and/or mutations that are prevalent in B-cell Non-Hodgkin Lymphoma, such as in LBCL) by targeted next generation sequencing assays including but not limited to clonoSEQ® MRD assay; (Adaptive Biotechnologies; Seattle, WA), Avenio (Roche Diagnostics; Basel, Switzerland) or PhasED-SEQ (Foresight Diagnostics; Aurora, CO).

Blood samples may be taken for assessment of ctDNA levels after administration of the antibody, or when the antibody is administered in multiple treatment cycles such as in 28-day treatment cycles as provided below, during said treatment cycles. The levels of ctDNA may be assessed to determine whether the amount of cancer DNA is declining as a potential measure of early response, e.g., based on log fold change from first day of treatment, e.g, on day 1 of cycle 1, when the antibody is administered in multiple treatment cycles. In certain embodiments, MRD negativity is determined by clone sequences being undetectable/below the limit of detection (LOD) of the assay. Duration of MRD negativity (MRD−) is defined as the number of days from the first documentation of MRD-to the date of MRD status change (not MRD−) or disease progression.

Particular embodiments of the invention provide methods, wherein said bispecific antibody is administered to said subject to provide a complete response and negative MRD status/MRD negativity.

In another aspect, the invention provides a method of decreasing a risk of relapse and/or disease progression in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
iii. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
iv. a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein said bispecific antibody is administered to provide a negative minimal residual disease (MRD) status/MRD negativity, wherein the negative MRD status/MRD negativity is indicative of a decreased risk of relapse and/or disease progression.

It is understood that when herein reference is made to methods of treatments, i.e. a method of treating a B-cell non-Hodgkin lymphoma (B-NHL) in a human subject, which method comprises administering subcutaneously to a human subject in need thereof, a bispecific antibody in accordance with the invention, which preferably is a full length antibody and/or comprises an inert Fc region, phrased differently, reference is made as well to bispecific antibodies in accordance with the invention, for use in the treatment of a B-cell non-Hodgkin lymphoma (B-NHL) in a human subject, which use comprises administering subcutaneously to a human subject in need thereof, a bispecific antibody in accordance with the invention, which preferably is a full length antibody and/or comprises an inert Fc region. Hence, wherein reference is made herein to methods for treatment of B-NHL with a bispecific antibody targeting CD3 and CD20 as defined herein, reference is also made to said bispecific antibody targeting CD3 and CD20 for use in the treatment of B-NHL in a human subject.

In a further aspect, the current invention provides for a method of treating a B-NHL, wherein said bispecific antibody comprises:
(i) a first binding arm comprising a first antigen-binding region binding to human CD3ε (epsilon), wherein said first antigen-binding region comprises a heavy chain variable (VH) region CDR1, CDR2, and CDR3 having the sequences as set forth in SEQ ID NOs. 1, 2, and 3, respectively, and a light chain variable (VL) region CDR1, CDR2, and CDR3 having the sequences as set forth in SEQ ID NO. 4, the sequence GTN, and the sequence as set forth in SEQ ID NO. 5, respectively, and
(ii) a second binding arm comprising a second antigen-binding region binding to human CD20, wherein said second antigen-binding region comprises a heavy chain variable (VH) region CDR1, CDR2, and CDR3 having the sequences as set forth in SEQ ID NOs. 8, 9, and 10, respectively, and a light chain variable (VL) region CDR1, CDR2, and CDR3 having the sequences as set forth in SEQ ID NO. 11, the sequence DAS, and the sequence as set forth in SEQ ID NO. 12, respectively.

In still a further aspect, the current invention provides for a method of treating a B-NHL, wherein said bispecific antibody comprises:
(i) a first binding arm comprising a first antigen-binding region binding to human CD3ε (epsilon), comprising a variable heavy chain region and a variable light chain region wherein the variable heavy chain region comprises SEQ ID NO: 6; and wherein the variable light chain region comprises SEQ ID NO: 7; and
(ii) a second binding arm comprising a second antigen-binding region binding to human CD20, comprising a variable heavy chain region and a variable light chain region wherein the variable heavy chain region comprises SEQ ID No: 13; and wherein the variable light chain region comprises SEQ ID No: 14.

Non-Hodgkin Lymphoma's (NHLs) represent a disease entity characterized by malignant transformation of the cells from lymphoid tissue. NHLs of B-cell origin (“B-NHL” or “B-cell NHL”) as defined herein constitute a diverse set of neoplasms within the larger context of NHL. These are diagnosed, and distinction of B-NHL subtypes determined, using standard classification criteria by a pathologist (based on tissue biopsy), including morphologic features by histology, surface markers (immunohistochemistry/flow cytometry), chromosomal abnormalities/translocations (karyotyping, fluorescence in situ hybridization (FISH)), and molecular (gene mutation) findings. B-NHLs are diagnosed and classified based NL, et al. WHO Classification of Tumours of Haematopoietic and Lymphoid Tissues (Revised ed. 4th). Lyon, France: IARC Press (2017); Swerdlow S H, Campo E, Harris N L, et al. WHO Classification of Tumours of Haematopoietic and Lymphoid Tissues (ed. 4th). Lyon, France: IARC Press (2008)).

Briefly, B-NHLs are typically divided into indolent (slow-growing) and aggressive subtypes. Aggressive B-NHLs have high Ki67 expression, whereas indolent B-NHLs have relatively low Ki67 expression. As a generalization, indolent lymphomas respond to treatment and are kept under control (in remission) with long-term survival of many years but are not cured. Aggressive lymphomas usually require intensive treatments, with some having a good prospect for a permanent cure. Aggressive B-NHL includes: Diffuse large B-cell lymphoma (DLBCL), high-grade B-cell lymphoma (HGBCL), primary mediastinal large B-cell lymphoma (PMBCL), follicular lymphoma (FL) grade 3B, mantle cell lymphoma (MCL). Indolent B-NHL includes FL grades 1-3A, marginal-zone lymphoma (MZL) and small lymphocytic lymphoma (SLL). Diffuse large B-cell lymphoma (DLBCL) is the most common type of NHL accounting for approximately 30% to 40% of all NHL diagnoses, followed by FL (20% to 25% of all NHL diagnoses). The majority of the B-cell lymphomas express B-cell markers, such as CD19, CD20, CD22, and CD79b. The biologic heterogeneity of B-cell malignancies is reflected in the clinical course and outcome of individual diseases. Indolent diseases such as FL G1-3A, MZL, and SLL evolve slowly, with a median survival of 8 to 10 years. In contrast, more aggressive diseases such as DLBCL/HGBCL, if left untreated, have a median survival of 6 months. The median age at diagnosis for most patients with lymphoma is approximately 60 to 65 years (WHO, 2008).

Hence, the method in accordance with the invention further comprises the treatment of B-NHL, wherein said B-NHL is selected from the group consisting of diffuse large B-cell lymphoma (DLBCL), high-grade B-cell lymphoma (HGBCL), primary mediastinal large B-cell lymphoma (PMBCL), mantle cell lymphoma (MCL), follicular lymphoma (FL), marginal-zone lymphoma (MZL) and small lymphocytic lymphoma (SLL). The HGBCL may be selected from HGBCL with MYC and BCL2 and/or BCL6 rearrangement and HGBCL, not otherwise specified (NOS).

In another aspect, the method in accordance with the invention as provided herein comprises the treatment of FL, HGBCL, or DLBCL. In yet another aspect, the method in accordance with the invention as provided herein comprises the treatment of FL. In another aspect, the method in accordance with the invention as provided herein comprises the treatment of HGBCL. In still another aspect, the method in accordance with the invention as provided herein comprises the treatment of DLBCL. In another further aspect, the method in accordance with the invention as provided herein comprises the treatment of HGBCL and/or DLBCL. In yet another aspect, the method in accordance with the invention as provided herein comprises the treatment of FL, MCL, HGBCL, or DLBCL. In another aspect, the method in accordance with the invention as described herein comprises the treatment of MCL.

“DLBCL/HGBCL” or “DLBCL and HGBCL” or, “DLBCL or HGBCL” as defined herein refers to B-NHL classified as either diffuse large B-cell lymphoma or high-grade B-cell lymphoma, in accordance with the WHO classification as defined in Swerdlow S H, Campo E, Harris N L, et al. WHO Classification of Tumours of Haematopoietic and Lymphoid Tissues (ed. 4th). Lyon, France: IARC Press (2008) and Swerdlow S H, Campo E, Harris N L, et al. WHO Classification of Tumours of Haematopoietic and Lymphoid Tissues (Revised ed. 4th). Lyon, France: IARC Press (2017), which are incorporated herein by reference.

“FL”, i.e. follicular lymphoma, as defined herein refers to B-NHL which originates from specific types of B-cells known as centrocytes and centroblasts that typically form follicular or follicle-like structures. FL typically has a slow disease course which persists essentially unchanged for years. FL can be classified in accordance with the WHO classification as defined in Swerdlow S H, Campo E, Harris N L, et al. WHO Classification of Tumours of Haematopoietic and Lymphoid Tissues (ed. 4th). Lyon, France: IARC Press (2008) and Swerdlow S H, Campo E, Harris N L, et al. WHO Classification of Tumours of Haematopoietic and Lymphoid Tissues (Revised ed. 4th). Lyon, France: IARC Press (2017), which are incorporated herein by reference.

“MCL”, i.e. Mantle cell lymphoma, comprises B-cell lymphoma with chromosomal translocation t (11; 14) leading to expression of cyclin D1, also including CD5+. MCL as defined herein includes B-NHL classified as MCL in accordance with the WHO classification as defined in Swerdlow S H, Campo E, Harris N L, et al. WHO Classification of Tumours of Haematopoietic and Lymphoid Tissues (ed. 4th). Lyon, France: IARC Press (2008) and

Swerdlow S H, Campo E, Harris N L, et al. WHO Classification of Tumours of Haematopoietic and Lymphoid Tissues (Revised ed. 4th). Lyon, France: IARC Press (2017), which are incorporated herein by reference.

In some embodiments, the B-NHL is a large B-cell lymphoma (LBCL); e.g. selected from diffuse large B-cell lymphoma (DLBCL), high-grade B-cell lymphoma (HGBCL), primary mediastinal large B-cell lymphoma (PMBCL), and follicular lymphoma grade 3B (FL G3B).

In further embodiments, the human subject has relapsed or refractory large B-cell lymphoma and has received two or more lines of systemic therapy.

In some embodiments, the B-NHL is selected from the group consisting of diffuse large B-cell lymphoma (DLBCL), high-grade B-cell lymphoma (HGBCL), primary mediastinal large B-cell lymphoma (PMBCL), mantle cell lymphoma, follicular lymphoma (FL), marginal-zone lymphoma and small lymphocytic lymphoma.

In some embodiments, the B-NHL is FL, HGBCL, or DLBCL.

In some embodiments, the B-NHL is aggressive B-cell non-Hodgkin lymphoma (aNHL).

In some embodiments, the B-NHL is double-hit or triple-hit lymphoma; such as HGBCL with MYC, BCL2 and/or BCL6 gene rearrangements).

In some embodiments, the LBCL has transformed from another indolent lymphoma or from chronic lymphocytic leukemia (CLL).

In some embodiments, the subject has received treatment of said BNHL prior to the treatment with said bispecific antibody.

In some embodiments, the subject has received 1, 2, 3, 4, 5 or 6 prior lines of treatment of said B-NHL.

In some embodiments, the subject has received prior treatment which is chimeric antigen receptor T (CAR-T) cell therapy.

In some embodiments, the subject is refractory to, or has relapsed from said prior treatment.

In some embodiments, the subject is refractory to, or has relapsed from last prior treatment.

In some embodiments, the last prior treatment received by said subject is which is chimeric antigen receptor T (CAR-T) cell therapy.

In some embodiments, the subject has received prior to the treatment with the bispecific antibody a treatment with a CD20 monospecific antibody, such as rituximab. In some embodiments, the said treatment with the CD20 monospecific antibody, the cancer relapsed. In some embodiments, the treatment with the CD20 monospecific antibody, the cancer was refractory to said treatment. In some embodiments, the CD20 monospecific antibody was used in a combination treatment.

A further aspect of the invention provides a method of treating relapsed/refractory large B-cell lymphoma in a human subject, the method comprising administering to said subject, a bispecific antibody at a dose of at least 24 mg, said bispecific antibody being a full-length antibody, that comprises
i. a first binding arm comprising a first antigen-binding region binding to human CD38 (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein the subject achieves a complete response and a minimal residual disease (MRD) negativity.

In some embodiments relating to this and other aspects of the invention, the subject is in complete response and is minimal residual disease (MRD) negative, when the antibody has been administered for 6 months or more, such as when the antibody has been administered for 7 months or more, 8 months or more, 9 months or more, 10 months or more, 11 months or more or such as for 12 months or more.

In other embodiments, the invention provides methods, wherein when the antibody has been administered to the subject for 9 months:
the probability of progression free survival of said subject is 60% or more, such as 65% or more 70% or more, 75% or more, or such as 80% or more; and
the subject is MRD negative.

In particular, the invention provides methods, wherein when the antibody has been administered to the subject for 10 months:
the probability of progression free survival of said subject is 60% or more, such as 65% or more 70% or more, 75% or more, or such as 80% or more; and
the subject is MRD negative.

In particular, the invention also provides methods, wherein when the antibody has been administered to the subject for 11 months:
the probability of progression free survival of said subject is 60% or more, such as 65% or more 70% or more, 75% or more, or such as 80% or more; and
the subject is MRD negative.

In particular, the invention provides methods, wherein when the antibody has been administered to the subject for 12 months:
the probability of progression free survival of said subject is 60% or more, such as 65% or more 70% or more, 75% or more, or such as 80% or more; and
the subject is MRD negative.

In some embodiments, the subject has primary refractory disease and achieves a complete response and a minimal residual disease (MRD) negativity.

In other embodiments, the subject is CAR T-naïve and achieves a complete response and a minimal residual disease (MRD) negativity.

In other embodiments, the subject has primary refractory disease and achieves a complete response and a minimal residual disease (MRD) negativity.

In still other embodiments, the subject is refractory to prior CAR T treatment and achieves a complete response and a minimal residual disease (MRD) negativity.

In further embodiments, the subject has DH/TH rearrangements and achieves a complete response and a minimal residual disease (MRD) negativity.

In some embodiments, the patient received further prior lines of treatment for said B-cell NHL.

With regard to the dose of at least 24 mg of the bispecific antibody that is to be administered, or any other specified dose, it is understood that this amount refers to the amount of a bispecific antibody representing a full-length antibody, such as epcoritamab as defined in the example section. Hence, phrased differently, one may refer to administering a dose of a bispecific antibody of at least 24 mg as administering a dose of a bispecific antibody in accordance with the invention, wherein said dose corresponds to a dose of at least 24 mg dose of epcoritamab. One can determine the amount of antibody to be administered when for example, antibodies would be used differing substantially in molecular weight from the molecular weight of a full-length antibody such as epcoritamab. The amount of antibody thus required as defined herein can be calculated by dividing the molecular weight of the antibody by the weight of a full-length antibody such as epcoritamab and multiplying the outcome thereof with the specified dose as described herein. As long as the bispecific antibody has highly similar features as Duobody CD3×CD20, with regard to plasma half-life, Fc inertness, and/or binding region features for CD3 and CD20, i.e. with regard to CDRs and epitope binding features, e.g. a functional variant of Duobody CD3×CD20, such antibodies may be contemplated in accordance with the invention and administered at a dose corresponding with the dose as defined herein for a full-length antibody such as epcoritamab.

As said, the bispecific antibody binding to CD3 and CD20 is to be administered subcutaneously. The bispecific antibody hence is formulated in a pharmaceutical composition such that it is compatible with subcutaneous (s.c.) administration, i.e. having a formulation and/or concentration that allows pharmaceutical acceptable s.c. administration at the selected doses as defined herein. Preferably subcutaneous administration is carried out by injection. For example, formulations for Duobody CD3×CD20 that are compatible with subcutaneous formulation that can be contemplated have been described (e.g. as described in WO2019155008, which is incorporated herein by reference). Preferred formulations for the bispecific antibody in accordance with the invention may be formulated using sodium acetate trihydrate, acetic acid, sorbitol, polysorbate 80, water for injection, having a pH of 5.5.

A suitable pharmaceutical composition for the bispecific antibody in accordance with the invention can comprise the bispecific antibody, 20-40 mM acetate, 140-160 mM sorbitol, and a surfactant, such as polysorbate 80, and having a pH of 5.3-5.6. A suitable pharmaceutical formulation for the bispecific antibody in accordance with the invention may comprise an antibody concentration in the range of 5-100 mg/ml, e.g. 5 mg/mL, 48 mg/mL or 60 mg/ml of the bispecific antibody, 30 mM acetate, 150 mM sorbitol, 0.04% w/v polysorbate 80, and having a pH of 5.5. Such a formulation may be appropriately diluted with e.g. the formulation buffer to allow proper dosing and subcutaneous administration. In any case, the volume of the pharmaceutical composition is appropriately selected to allow for subcutaneous administration of the antibody. For example, the volume to be administered is in the range of about 0.3 mL to about 3 mL, such as from 0.3 mL to 3 mL. The volume to be administered can be 0.5 mL, 0.8 mL, 1 mL, 1.2 mL, 1.5 ml, 1.7 mL, 2 mL, or 2.5 mL. The volume to be administered can be 0.5 mL. The volume to be administered can be 0.8 mL. The volume to be administered can be 1.2 mL. The volume to be administered can be 1.5 mL. The volume to be administered can be 1.7 mL. The volume to be administered can be 2 mL. The volume to be administered can be 2.5 mL. In one embodiment the preferred volume for s.c. administration is about 1 mL. In another embodiment the preferred volume for s.c. administration is 1 mL.

As said, the methods (or uses of CD3×CD20 antibodies) in accordance with the invention are for the treatment of human patients suffering from B-NHL. It is understood that the method in accordance with the invention may be the first, or may be part of the first treatment provided to such patients. However, patients may have been subjected to prior treatments of B-NHL. Prior treatments may include, one or more of chemotherapy, radiation therapy, immunotherapy, and targeted therapy, or combination hereof, but not may not be restricted thereto. Most commonly the standard of care comprises treatments with CD20 monoclonal antibodies, alkylating agents, and anthracycline, either alone or in combination. It is understood that methods and uses in accordance with the invention may also be used in combination with other suitable treatments.

For example, the most common chemotherapy combination for the first treatment of aggressive B-NHL is called CHOP and contains 4 medications: cyclophosphamide, doxorubicin, vincristine, and prednisone. Adding an anti-CD20 monoclonal antibody, such as rituximab, has been shown to be an improvement over CHOP alone, also referred to as R-CHOP. Induction with R-CHOP is standard of care in 1st line treatment of DLBCL, as well as being one of more available 1^{st} standard of care treatments in FL. In DLBCL, 2^{nd} line treatments include intensive salvage treatment (rituximab/dexamethasone/high-dose cytarabine/cisplatin [R-DHAP], rituximab/ifosfamide/carboplatin/etoposide [R-ICE], or rituximab/gemcitabine/dexamethasone/cisplatin [R-GDP]) followed by, if chemosensitive, high-dose chemotherapy with autologous hematopoietic stem cell transplantation (HDT-ASCT). For DLBCL patients not being eligible to intensive salvage treatment and HDT-ASCT due to age or comorbidities, 2^{nd} line treatments include rituximab/gemcitabine/oxaliplatin (R-GemOx) and rituximab/bendamustine (RB). There is no clear standard of care for DLBCL late-line relapse, but interventions include allogeneic hematopoietic stem cell transplantation, lenalidomide, ibrutinib, and chimeric antigen receptor T (CAR-T) cell therapy (Chavez et al., Best Pract Res Clin Haematol, 2018 June; 31 (2): 135-146).

Hence, in a further embodiment, in a method in accordance with the invention, a human subject having B-NHL has received at least one line of treatment prior to the treatment in accordance with the invention. In another embodiment, a human subject having B-NHL has received one line of treatment prior to the treatment in accordance with the invention. In another further embodiment, a human subject having B-NHL has received two lines of treatment prior to the treatment in accordance with the invention. In still another further embodiment, a human subject having B-NHL has received three lines of treatment prior to the treatment in accordance with the invention. In yet another further embodiment, a subject having B-NHL has received more than three lines of treatment prior to the treatment in accordance with the invention. In another further embodiment, a subject having B-NHL has received one, two, three, or more lines of treatment prior to the treatment in accordance with the invention. In a further embodiment, a prior line of treatment comprises R-CHOP. In yet another further embodiment, a prior line treatment comprises a CAR-T cell therapy.

Human subjects having B-NHL will generally have disease that is positive for CD19 and CD20. Hence, prior cancer treatments such human subjects may have received include anti-CD20 mAbs, and may also include engineered T cells targeting CD19, e.g. a CAR-T therapy (Schuster S J, Bishop M R, Tam C S, et al. Tisagenlecleucel in adult relapsed or refractory diffuse large B-cell lymphoma. N Engl J Med. 2019; 380 (1): 45-56). During such treatments, or any other treatments, the B-NHL may be refractory or have relapsed to said treatment. Hence, in one aspect of the invention, said human subject has received prior to the treatment with the bispecific antibody a treatment with a CD20 monospecific antibody, such as rituximab. Furthermore, during said prior treatment with the CD20 monospecific antibody, the B-NHL cancer relapsed, or, the B-NHL cancer was refractory to said treatment. Such prior treatment with a CD20 monospecific antibody may be a treatment wherein the CD20 monospecific antibody was used in a combinatorial approach.

In a further embodiment, said bispecific antibody is administered at a dose of at least 40 mg. In another further embodiment, said bispecific antibody is administered at a dose in the range of between 30 mg to 100 mg, or between 35 mg and 90 mg. More preferably, in another further embodiment, said bispecific antibody is administered at a dose in the range of between 40 mg and 70 mg. In another further embodiment, said bispecific antibody is administered at a dose of at least 48 mg. In another further embodiment, said bispecific antibody is administered at a dose of at least 60 mg. It may also be contemplated to administer said bispecific antibody at a dose of 60 mg. In another embodiment, said bispecific antibody is administered at a dose of 72 mg. In yet another further embodiment, said bispecific antibody is administered at a dose of 84 mg.

In still a further embodiment, said bispecific antibody is administered at a dose of 40, 41, 42, 43, 44, 45, 46, 47, 48, 49, 50, 51, 52, 53, 54, 55, 56, 57, 58, 59, 60, 61, 62, 63, 64, 65, 66, 67, or 69 mg. In a preferred embodiment, said bispecific antibody is administered at a dose of 48 mg.

Said dose preferably is administered weekly, and preferably said dose is administered (at least) 4 times. After said weekly administration, one may reduce the interval of administration to an administration once every two weeks, also referred to as biweekly administration. Such biweekly administration may be performed (at least) 6 times. After said biweekly administration, the interval may be reduced further to once every four weeks. Such administration every four weeks may be performed for an extended period.

It is understood that herein the said dose as referred herein above, may also be referred to as a full or a flat dose in the scenarios above wherein e.g. the said weekly dose, said dose every two weeks and/or said dose every four weeks, is administered is at the same level. Hence, when a dose of 48 mg is selected, preferably, at e.g. each weekly administration, each biweekly administration, and each administration every four weeks, the same dose of 48 mg is administered each time. Prior to administering said dose (to which can be referred as full dose, or flat dose) a priming or a priming and subsequent intermediate (second priming) dose may be administered. This may be advantageous as it may allow for effectively mitigating CRS risk and severity, which is a side-effect that can occur during treatment with the bispecific antibody binding both CD3 and CD20 in accordance with the invention. Such priming, or priming and intermediate doses, are at a lower dose as compared with the flat or full dose.

Hence, in the method or use in accordance with the invention, prior to administering said weekly dose, a priming dose of said bispecific antibody may be administered. Said priming dose preferably is administered one week prior to administering the first dose of said weekly dose. A priming dose may be selected which is in the range of 20-2000 μg, preferably in the range of 50-1000 μg, more preferably in the range of 70-350 μg. A priming dose that may be selected is 80, 100, 120, 140, 160, 180, 200, 220, 240, 260, 280, 300, 320, 350, 400, 450, 500, 600, 700, 800, 900, or 1000 μg. A preferred priming dose that may be selected is 160 μg of the full-length bispecific antibody. A further preferred priming dose that may be selected is 800 μg of the full-length bispecific antibody.

Also, in the method or use in accordance with the invention, after administering said priming dose and prior to administering said weekly dose, an intermediate dose of said bispecific antibody may be administered. Preferably, said priming dose is administered two weeks, and said intermediate dose is administered one week, before administering the first dose of said weekly dose. Said intermediate dose typically is selected from a range in between the priming dose and the flat or full dose. Said intermediate dose may be selected in the range of 200-8000 μg, or preferably in the range of 400-4000, more preferably in the range of 600-2000 μg. An intermediate dose that may be selected is 200, 300, 400, 500, 600, 700, 800, 900, 1000, 1100, 1200, 1300, 1400, 1500, or 1600 μg. A preferred intermediate dose that may be selected is 800 μg of the full-length bispecific antibody. A further preferred intermediate dose that may be selected is 4000 μg of the full-length bispecific antibody.

In some embodiments, the bispecific antibody is administered subcutaneously to the subject in 28-day cycles.

Accordingly, in another embodiment, advantageous dosing regimens that may be contemplated in the methods and uses of the invention may include administering the bispecific antibody subcutaneously in 28-day cycles, wherein on
a) Day 1, 8, 15 and 22 the first cycle, a priming dose is administered at day 1, an intermediate dose at day 8, and a dose of 48 mg at days 15, 22; for the first cycle; on
b) Day 1, 8, 15 and 22 of cycles 2-3, a dose of 48 mg is administered; on
c) Day 1, 15 of cycles 4-9, a dose of 48 mg is administered; and on
d) Day 1, of further subsequent cycles, a dose of 48 mg is administered.

In yet a further embodiment, an advantageous dosing regimen that may be contemplated in the methods and uses of the invention may include administering the bispecific antibody subcutaneously in 28-day cycles, wherein on
a) Day 1, 8, 15 and 22 the first cycle, a priming dose of 160 μg is administered at day 1, an intermediate dose of 800 μg at day 8, and a dose of 48 mg at days 15, 22; for the first cycle; on
b) Day 1, 8, 15 and 22 of cycles 2-3, a dose of 48 mg is administered;
c) Day 1, 15 of cycles 4-9, a dose of 48 mg is administered; and on
d) Day 1, of further subsequent cycles, a dose of 48 mg is administered.

In still another embodiment, an advantageous dosing regimen that may be contemplated in the methods and uses of the invention may include administering the bispecific antibody subcutaneously in 28-day cycles, wherein on
a) Day 1, 8, 15 and 22 the first cycle, a priming dose is administered at day 1, an intermediate dose at day 8, and a dose of 60 mg at days 15, 22; for the first cycle; on
b) Day 1, 8, 15 and 22 of cycles 2-3, a dose of 60 mg is administered; on
c) Day 1, 15 of cycles 4-9, a dose of 60 mg is administered; and on
d) Day 1, of further subsequent cycles, a dose of 60 mg is administered.

In yet another further embodiment, an advantageous dosing regimen that may be contemplated in the methods and uses of the invention comprises administering the bispecific antibody subcutaneously in 28-day cycles, wherein on
a) Day 1, 8, 15 and 22 the first cycle, a priming dose of 160 μg is administered at day 1, an intermediate dose of 800 μg at day 8, and a dose of 60 mg at days 15, 22; for the first cycle; on
b) Day 1, 8, 15 and 22 of cycles 2-3, a dose of 60 mg is administered;
c) Day 1, 15 of cycles 4-9, a dose of 60 mg is administered;
d) Day 1, of further subsequent cycles, a dose of 60 mg is administered.

In one embodiment, alternatively, on days 1 and 8 of the first cycle, a priming dose of 80 μg and an intermediate dose of 800 μg is selected. In one embodiment, alternatively, on days 1 and 8 of the first cycle, a priming dose of 80 μg and an intermediate dose of 1200 μg is selected. In one embodiment, alternatively, on days 1 and 8 of the first cycle, a priming dose of 80 μg and an intermediate dose of 1600 μg is selected. In another embodiment, on days 1 and 8 of the first cycle, a priming dose of 160 μg and an intermediate dose of 1200 μg is selected. In another embodiment, on days 1 and 8 of the first cycle, a priming dose of 160 μg and an intermediate dose of 1600 μg is selected.

As said, such uses and methods are advantageous for the treatment of B-NHL, more preferably in the treatment of FL, DLBCL and/or HGBCL. The uses and methods are further advantageous for treatment of large B-cell lymphoma, such as large B-cell lymphoma selected from the group consisting of DLBCL, HGBCL, PMBCL, and FL grade 3B (G3B), Said treatment in accordance with the current invention, is maintained continuously, using treatment regimens as outlined above. When progressive disease develops, unacceptable toxicity occurs, or by death of the patient, treatment may be terminated.

Cytokine release syndrome (CRS) can occur when means and methods are used in human subjects that utilize immune cell- and bi-specific antibody-based approaches that function by activation of immune effector cell, such as by engaging CD3 (Lee et al., Biol Blood Marrow Transplant. 2019 April; 25 (4): 625-638, which is incorporated herein by reference). Hence, CRS mitigation is preferred in the methods and uses in accordance with the invention. As part of CRS mitigation, the selection of a priming dose and/or intermediate dose is highly preferred prior to administering the at least 24 mg dose, i.e. a full dose or flat dose, in accordance with the invention. CRS can be classified in accordance with standard practice (e.g. as outlined in Lee et al., Biol Blood Marrow Transplant. 2019 April; 25 (4): 625-638, which is incorporated herein by reference). CRS may include excessive release of cytokines, for example of proinflammatory cytokines, e.g. IL-6, TNF-alpha or IL-8, that may result in adverse effects like fever, nausea, vomiting and chills. Thus, despite the unique anti-tumor activity of bispecific antibodies such as epcoritamab, their immunological mode of action may trigger unwanted “side” effects, i.e. in the induction of unwanted inflammatory reactions. Hence, patients may be further subjected to a concomitant treatment, prophylaxis and/or premedication with e.g. analgesics, antipyretics, and/or anti-inflammatory drugs to mitigate possible CRS symptoms.

In one embodiment, said human subjects in methods and uses in accordance with the invention are treated with prophylaxis for cytokine release syndrome. Preferably, such a prophylaxis includes the administration of a corticosteroid. Said prophylaxis in one embodiment is administered at the same day as the bispecific antibody. Said prophylaxis can be administered on the subsequent day as well, more preferably at subsequent days 2 and 3. Optionally, said prophylaxis may be further administered at day 4, It is understood that days 2, 3 and 4 when relating to further medication, such as prophylaxis, is relative to the administration of the bispecific antibody which is administered at day 1. Hence, when e.g. in a cycle on day 15 the antibody is administered, and prophylaxis is administered, said prophylaxis corresponding to days 2, 3 and 4 are days 16, 17, and 18 of the cycle. In a further embodiment, said prophylaxis is administered at the day when the bispecific antibody is administered and at subsequent days 2-3, and day 4 is optional. When said prophylaxis is administered at the same day as the bispecific antibody, said prophylaxis is preferably administered 30-120 minutes prior to said administration of the bispecific antibody. A corticosteroid that may be preferred is prednisolone, e.g. at an intravenous dose of 100 mg, or an equivalent thereof, including an oral dose.

Furthermore, in one embodiment, said human subjects in methods and uses in accordance with the invention are treated with premedication to reduce reactions to injections. In one embodiment, said premedication includes the administration of antihistamines. In another embodiment, said premedication includes the administration of antipyretics. In a further embodiment, said premedication includes systemic antihistamines and antipyretics. An antihistamine that may be selected is diphenhydramine, e.g. at an intravenous or oral dose 50 mg, or an equivalent thereof. An antipyretic that may be selected is acetaminophen, e.g. at an oral dose of 650-1000 mg, or equivalent thereof. More preferably, said premedication is administered at the same day as the bispecific antibody, most preferably prior to the injection with the bispecific antibody, such as 30-120 minutes prior to said administration of the bispecific antibody.

It is understood that said premedication and/or prophylaxis are to be administered at least in the initial phase of the treatment. More preferably, during the first four administrations of the bispecific antibody. For example, said prophylaxis can be administered as described hereinbefore, during the first 28 day cycle of the bispecific antibody administration. Said premedication is preferably administered as well during said first cycle.

Usually, risk of reactions during the initial treatment subside after a few administrations, e.g. after the first four administrations (first cycle). Hence, and when the human subject does not experience CRS, prophylaxis medication for CRS may be stopped. However, preferably, when the human subject would experience a CRS greater than grade 1, CRS prophylaxis may continue. Likewise, premedication may also optionally continue.

In a further embodiment, in methods and uses in accordance with the invention as described herein, said prophylaxis is administered during the second 28-day cycle when the human subject experiences CRS greater than grade 1 after the fourth administration of the bispecific antibody in cycle 1. Furthermore, said prophylaxis can be continued during a subsequent cycle, when in the last administration of the bispecific antibody of the previous cycle, the human subject experiences CRS greater than grade 1. Any premedication may be optionally administered during the second cycle. Further premedication may be optionally administered during subsequent cycles as well.

In one embodiment, premedication and prophylaxis for CRS is administered, including an antihistamine, said antihistamine may be diphenhydramine, e.g. at an intravenous or oral dose 50 mg, or an equivalent thereof, and an antipyretic, said antipyretic may be acetaminophen, e.g. at an oral dose of 650-1000 mg, or an equivalent thereof, and the administration of a corticosteroid, which corticosteroid may be prednisolone, e.g. at an intravenous dose of 100 mg, or an equivalent thereof, said premedication and prophylaxis is administered 30-120 minutes prior to administration of the bispecific antibody. On subsequent days 2, 3, and optionally day 4, further prophylaxis is administered comprising the systemic administration of a corticosteroid, which corticosteroid may be prednisolone, e.g. at an intravenous dose of 100 mg, or an equivalent thereof. Preferably, on subsequent days 2, 3, and 4, said further prophylaxis is administered comprising the systemic administration of a corticosteroid, which corticosteroid may be prednisolone, e.g. at an intravenous dose of 100 mg, or an equivalent thereof. Such premedication and prophylaxis schedule preferably is administered during the first four administrations of the bispecific antibody in accordance with the invention. For example, during the first 28-day cycle of bispecific antibody administration as described herein. Furthermore, subsequent cycles, in case of e.g. CRS greater than grade 1 occurring during the last administration of the prior cycle, can include the same administration schedule, wherein the premedication as part of the administration schedule is optional.

At the provided advantageous doses and/or treatment regimens as provided herein CRS can be well managed while at the same time allowing for effectively controlling and/or treating B-NHL. As shown in the example section, in the methods and uses as described herein, in said human subjects, manageable cytokine release syndrome CRS may occur. Human subjects receiving treatments in accordance with the invention may have CRS of grade 1 as defined in accordance with standard practice. Human subjects receiving treatments in accordance with the invention may have manageable CRS of grade 2 as defined in accordance with standard practice. Hence, human subjects receiving treatments in accordance with the invention may have manageable CRS of grade 1 or grade 2 during as defined in accordance with standard practice. In accordance with standard classification for CRS, a grade 1 CRS includes a fever to at least 38° C., no hypotension, no hypoxia, and a grade 2 CRS includes a fever to at least 38° C. plus hypotension, not requiring vasopressors and/or hypoxia requiring oxygen by low flow nasal cannula or blow by. Such manageable CRS can occur during cycle 1. Human subjects receiving treatments in accordance with the invention may also have CRS greater than grade 2 during said treatments as defined in accordance with standard practice. Hence, human subjects receiving treatments in accordance with the invention may also have CRS of grade 3 during said treatments as defined in accordance with standard practice. Such manageable CRS may further occur during cycle 1 and subsequent cycles.

Said human subjects may experience in methods and uses in accordance with the invention, pyrexia. Fatigue and injection site reactions may also occur. Human subjects may experience neurotoxicity, partial seizures, agraphia related to CRS, or confusional state related to CRS. Moreover, said human subjects may be observed not to experience tumor lysis syndrome.

Because of the safety profile of epcoritamab, it may not be required to hospitalize patients to administer epcoritamab. In a further embodiment, the methods and uses in accordance with the invention for the treatment of B-NHL involves the administration of epcoritamab in an outpatient setting. Administration in an outpatient setting does not involve hospitalization. In another embodiment, patients are hospitalized for administration only of the first full dose. Said first full dose is administered for example on day 15 of cycle 1, wherein days 1 and 8, respectively involve step up doses, as described herein. A preferred dose, in methods and uses as described herein, for outpatient use of epcoritamab includes the full dose, which may be up to 60 mg. More preferably, a dose for administration in outpatient use is 48 mg. In another embodiment, the methods and uses in accordance with the invention for the treatment of B-NHL involves the administration of epcoritamab exclusively in an outpatient setting.

As described herein, in the methods and uses in accordance with the invention, the bispecific antibody in accordance with the invention aimed at targeting CD3 and CD20 comprises:
(i) a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
(ii) a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14.

CDR1, CDR2 and CDR3 regions can be identified from variable heavy and light chain regions using methods known in the art. The CDR regions from said variable heavy and light chain regions can be annotated according to IMGT (see Lefranc M P. et al., Nucleic Acids Research, 27, 209-212, 1999] and Brochet X. Nucl. Acids Res. 36, W503-508 (2008)). Hence, also disclosed in the methods and uses in accordance with the invention, the bispecific antibody in accordance with the invention aimed at targeting CD3 and CD20 comprises:
(i) a first binding arm comprising a first antigen-binding region binding to human CD3ε (epsilon), wherein said first antigen-binding region comprises a heavy chain variable (VH) region CDR1, CDR2, and CDR3 having the sequences as set forth in SEQ ID NOs. 1, 2, and 3, respectively, and a light chain variable (VL) region CDR1, CDR2, and CDR3 having the sequences as set forth in SEQ ID NO. 4, the sequence GTN, and the sequence as set forth in SEQ ID NO. 5, respectively, and
(ii) a second binding arm comprising a second antigen-binding region binding to human CD20, wherein said second antigen-binding region comprises a heavy chain variable (VH) region CDR1, CDR2, and CDR3 having the sequences as set forth in SEQ ID NOs. 8, 9, and 10, respectively, and a light chain variable (VL) region CDR1, CDR2, and CDR3 having the sequences as set forth in SEQ ID NO. 11, the sequence DAS, and the sequence as set forth in SEQ ID NO. 12, respectively.

In yet a further embodiment, in the methods and uses in accordance with the invention, the bispecific antibody comprises
(i) a first binding arm comprising a first antigen-binding region binding to human CD38 (epsilon), comprising a variable heavy chain region and a variable light chain region wherein the variable heavy chain region comprises SEQ ID NO: 6; and wherein the variable light chain region comprises SEQ ID NO: 7; and
(ii) a second binding arm comprising a second antigen-binding region binding to human CD20, comprising a variable heavy chain region and a variable light chain region wherein the variable heavy chain region comprises SEQ ID No: 13; and wherein the variable light chain region comprises SEQ ID No: 14.

As said, the bispecific antibody in accordance with the invention is most preferably a full-length antibody and may have an inert Fc region. More preferably, the first binding arm for CD3 is derived from a humanized antibody, preferably from a full-length IgG1,λ (lambda) antibody such as H1L1 described in WO2015001085, which is incorporated herein by reference) and/or the second binding arm for CD20 is derived from a human antibody, preferably from a full-length IgG1,κ (kappa) antibody such as clone 7D8 as described in WO2004035607, which is incorporated herein by reference. The bispecific antibody may be produced from two half molecule antibodies. Each of the two half molecule antibodies comprising e.g. the respective first and second binding arms as listed herein in SEQ ID NOs. 6 and 7, and SEQ ID NOs. 13 and 14. The half-antibodies may be produced in CHO cells and the bispecific antibodies generated by e.g. Fab-arm exchange. In one embodiment, said bispecific antibody in accordance with the invention is a functional variant of Duobody CD3×CD20.

Various constant regions or variants thereof may be contemplated in accordance with the invention. Nevertheless, most preferably, the bispecific antibody in accordance with the invention comprises a human IgG1 constant region, such as a human IgG1 constant region as defined in SEQ ID NO. 15, or any other suitable IgG1 allotype. As said, the first binding arm of the bispecific antibody preferably is derived from a humanized antibody, preferably from a full-length IgG1,λ (lambda) antibody, hence comprising a λ light chain constant region. More preferably, said first binding arm as defined herein comprises a λ light chain constant region as defined in SEQ ID NO.22. Furthermore, said second binding arm of the bispecific antibody is derived from a human antibody, preferably from a full-length IgG1,κ (kappa) antibody, hence comprising a k light chain constant region. More preferably, said second binding arm comprises a k light chain constant region as defined in SEQ ID NO.23.

It is understood that the constant region, as it is part of a bispecific antibody, said constant region may comprise modifications to allow for efficient formation/production of bispecific antibodies and/or provide for an inert Fc region. Such modifications are I known in the art.

Different formats and uses of bispecific antibodies are known in the art (reviewed by Kontermann; Drug Discov Today, 2015 July; 20 (7): 838-47 and; MAbs, 2012 March-April; 4 (2): 182-97) and not necessarily be limited to any particular bispecific format or method of producing it. For example, bispecific antibodies may include but are not limited to (i) bispecific antibodies with complementary CH3 domains to force heterodimerization;), the so-called Knobs-into-Holes molecules (Genentech, WO9850431), CrossMAbs (Roche, WO2011117329) or electrostatically-matched molecules (Amgen, EP1870459 and WO2009089004; Chugai, US201000155133; Oncomed, WO2010129304).

Preferably, the bispecific antibody of the invention comprises an Fc-region comprising a first heavy chain with a first Fc sequence comprising a first CH3 region, and a second heavy chain with a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. More details on these interactions and how they can be achieved are provided in e.g. WO2011131746 and WO2013060867 (Genmab), which are hereby incorporated by reference. In a preferred embodiment, the bispecific antibody in the methods and uses in accordance with the invention comprises in said first heavy chain (i) the amino acid L in the position corresponding to F405 in a human IgG1 heavy chain of SEQ ID NO: 15, and comprises in said second heavy chain the amino acid R in the position corresponding to K409 in a human IgG1 heavy chain of SEQ ID NO: 15 is R, or vice versa.

Antibodies according to the present invention may comprise modifications in the Fc region to render the Fc region of the antibody inert, or non-activating. Hence, the bispecific antibodies disclosed herein, one or both heavy chains may be modified so that the antibody induces Fc-mediated effector function to a lesser extent relative to the bispecific antibody which does not have such modification. The Fc-mediated effector function may be measured by determining Fc-mediated CD69 expression on T cells (i.e. CD69 expression as a result of CD3 antibody-mediated, Fcγ receptor-dependent CD3 crosslinking), by binding to Fcγ receptors, by binding to C1q, or by induction of Fc-mediated cross-linking of FcγRs. In particular, the heavy chain constant sequences may be modified so that the Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g. as described in Example 3 of WO2015001085. Modifications of the heavy and light chain constant sequences may also result in reduced binding of C1q to said antibody. As compared to an unmodified antibody the reduction may be by at least 70%, at least 80%, at least 90%, at least 95%, at least 97%, or 100% and the C1q binding may be determined by ELISA. Further, the Fc region which may be modified so that said antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay. Examples of amino acid positions that may be modified, e.g. in an IgG1 isotype antibody, include positions L234 and L235. Hence, the antibody according to the invention may comprises a first and a second heavy chain, and wherein in both the first and the second heavy chain, the amino acid residues at the positions corresponding to positions L234 and L235 in a human IgG1 heavy chain according to Eu numbering are F and E, respectively. In addition, a D265A amino acid substitution can decrease binding to all Fcγ receptors and prevent ADCC (Shields et al., 2001, J. Biol. Chem. (276): 6591-604). Therefore, the antibody according to the invention may comprise a first and a second heavy chain, wherein in both the first and the second heavy chain, the amino acid residue at the position corresponding to position D265 in a human IgG1 heavy chain according to Eu numbering is A.

Most preferred, in the methods and uses in accordance with the invention, bispecific antibodies are provided wherein said first and second heavy chains, the amino acids in the positions corresponding to positions L234, L235, and D265 in a human IgG1 heavy chain, are F, E, and A, respectively. An antibody having these amino acids at these positions, is an example of an antibody having an inert Fc region, or a non-activating Fc region.

In the present application antibodies, which have the combination of three amino acid substitutions L234F, L235E and D265A and in addition the K409R or the F405L mutation disclosed herein above may be termed with the suffix “FEAR” or “FEAL”, respectively.

As said, an amino acid sequence of a wild type IgG1 heavy chain constant region is identified herein as SEQ ID NO: 15. Consistent with the embodiments disclosed above, the antibody of the invention may comprise an IgG1 heavy chain constant region carrying the F405L substitution and may have the amino acid sequence set forth in SEQ ID NO: 17 and/or an IgG1 heavy chain constant region carrying the K409R substitution and may have the amino acid sequence set forth in SEQ ID NO: 18, and have further substitutions that render the Fc region inert or non-activating. Hence, most and highly preferred, a combination of IgG1 heavy chain constant regions that are comprised in a bispecific antibody in accordance with the invention are an amino acid sequence of an IgG1 heavy chain constant region carrying the L234F, L235E, D265A and F405L substitutions such as identified herein as SEQ ID NO: 19 and an amino acid sequence of an IgG1 heavy chain constant region carrying the L234F, L235E, D265A and K409R substitutions such as identified herein as SEQ ID NO: 20.

In one further highly preferred embodiment, the bispecific antibody in the methods and uses as described herein comprises a first binding arm having a heavy chain and a light chain as defined in SEQ ID NOs. 24 and 25, respectively, and a second binding arm having a heavy chain and a light chain as defined in SEQ ID NOs. 26 and 27. Such an antibody may be referred to herein as Duobody CD3×CD20. Also, variants of such antibodies can be contemplated for the methods and uses as described herein. In still a further embodiment, the bispecific antibody in accordance with the invention is epcoritamab (CAS 2134641-34-0), or a biosimilar thereof.

In a further aspect, the invention provides a method of predicting a likelihood of relapse and/or disease progression in a subject having B-cell Non-Hodgkin Lymphoma (B-NHL), the method comprising: Measuring a minimal residual disease (MRD) status in the subject, wherein the subject receives or has received a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID No: 13; and wherein the variable light chain region comprises the CDR1, CDR2 and CDR3 sequences of SEQ ID No: 14;
wherein a positive MRD status is indicative of a likelihood of relapse and/or disease progression.

It will be understood that in relation to this aspect of the invention, the B-NHL is as defined above. Also, the subject has received prior treatment as defined in any one of claims 18 to 28. The bispecific antibody which is administered to the subject according to this aspect of the invention is preferably as defined above.

It will further be understood that the subject may treated with prophylaxis for cytokine release syndrome (CRS) as defined above. Likewise, the subject may be treated with premedication, e.g. to reduce reactions to injections, as defined in any one of the aspects and embodiments above.

Finally, it will be understood that the bispecific antibody administered according to this aspect may have any and all of the features defined in relation to other aspects of the invention.

### Further Embodiments

1. A method of treating a B-cell Non-Hodgkin Lymphoma (B-NHL) in a human subject, the method comprising administering subcutaneously to a human subject in need thereof, a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to provide a response in said subject, the response having a duration of 6 months or longer.

2. The method in accordance with embodiment 1, wherein the response has a duration of 7 months or longer, such as 8 months or longer, 9 months or longer, 10 months or longer, 11 months or longer, or such as a duration of 12 months or longer.

3. The method in accordance with embodiment 1 or 2, wherein said bispecific antibody is administered to the subject over a period which is 6 months or longer, such as 7 months or longer, such as 8 months or longer, 9 months or longer, 10 months or longer, 11 months or longer, or such as a duration of 12 months or longer.

4. The method in accordance with any one of the preceding embodiments, wherein said response comprises at least a partial response (PR), preferably wherein said response comprises a complete response.

5. The method in accordance with any one of the preceding embodiments, wherein said response comprises a complete response, and the response has a duration of 8 months or longer, such as a duration of 9 months or longer, 10 months or longer, 11 months or longer, 12 months or longer, 13 months or longer, 14 months or longer, 15 months or longer, 16 months or longer, 17 months or longer, 18 months or longer, 19 months or longer or such as 20 months or longer.

6. The method in accordance with any one of embodiments 1 to 5, wherein said subject shows a best overall response (BOR) of partial response (PR).

7. The method in accordance with any one of embodiments 1 to 5, wherein said subject shows a best overall response (BOR) of complete response.

8. The method in accordance with any one of embodiments 1 to 5 and 7, wherein said subject shows a complete response, and the response has a duration of 8 months or longer, such as a duration of 9 months or longer, 10 months or longer, 11 months or longer, 12 months or longer, 13 months or longer, 14 months or longer, 15 months or longer, 16 months or longer, 17 months or longer, 18 months or longer, 19 months or longer or such as 20 months or longer.

9. A method of achieving negative minimal residual disease (MRD) status/MRD negativity in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to said subject for a time and/or a number of treatment cycles sufficient to provide a negative minimal residual disease (MRD) status/MRD negativity.

10. The method in accordance with any one of embodiments 1 to 5 and 7 to 9, wherein said bispecific antibody is administered to said subject to provide a complete response and negative MRD status/MRD negativity.

11. A method of decreasing a risk of relapse and/or disease progression in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
i. a first binding arm comprising a first antigen-binding region binding to human CD38 (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to provide a negative minimal residual disease (MRD) status/MRD negativity, wherein the negative MRD status/MRD negativity is indicative of a decreased risk of relapse and/or disease progression.

12. The method in accordance with any one of the preceding embodiments, wherein the said B-NHL is a large B-cell lymphoma (LBCL); e.g. selected from diffuse large B-cell lymphoma (DLBCL), high-grade B-cell lymphoma (HGBCL), primary mediastinal large B-cell lymphoma (PMBCL), and follicular lymphoma grade 3B (FL G3B).

13. The method in accordance with any one of the preceding embodiments, wherein the human subject has relapsed or refractory large B-cell lymphoma and has received two or more lines of systemic therapy.

14. The method in accordance with any one of the preceding embodiments, wherein said B-NHL is selected from the group consisting of diffuse large B-cell lymphoma (DLBCL), high-grade B-cell lymphoma (HGBCL), primary mediastinal large B-cell lymphoma (PMBCL), mantle cell lymphoma, follicular lymphoma (FL), marginal-zone lymphoma and small lymphocytic lymphoma.

15. The method in accordance with any one of the preceding embodiments, wherein said B-NHL is FL, HGBCL, or DLBCL.

16. The method in accordance with any one of the preceding embodiments, wherein the B-NHL is aggressive B-cell non-Hodgkin lymphoma (aNHL).

17. The method in accordance with any one of the preceding embodiments, wherein said B-NHL is double-hit or triple-hit lymphoma; such as HGBCL with MYC, BCL2 and/or BCL6 gene rearrangements).

18. The method in accordance with any one of embodiments 13 to 17, wherein the LBCL has transformed from another indolent lymphoma or from chronic lymphocytic leukemia (CLL)

19. The method in accordance with any one of the preceding embodiments, wherein said subject has received treatment of said BNHL prior to the treatment with said bispecific antibody.

20. The method in accordance with embodiment 19, wherein said subject has received 1, 2, 3, 4, 5 or 6 prior lines of treatment of said B-NHL.

21. The method in accordance with embodiment 20, wherein said subject has received prior treatment which is chimeric antigen receptor T (CAR-T) cell therapy.

22. The method in accordance with any one of embodiments 19 to 21, wherein said subject is refractory to, or has relapsed from said prior treatment.

23. The method in accordance with any one of embodiments 19 to 22, wherein said subject is refractory to, or has relapsed from last prior treatment.

24. The method in accordance with any one of embodiments 21 to 23, wherein last prior treatment received by said subject is chimeric antigen receptor T (CAR-T) cell therapy.

25. The method in accordance with any one of the preceding embodiments, wherein said subject has received prior to the treatment with the bispecific antibody a treatment with a CD20 monospecific antibody, such as rituximab.

26. The method in accordance with embodiment 25, wherein during said treatment with the CD20 monospecific antibody, the cancer relapsed.

27. The method in accordance with embodiment 25 or 26, wherein during said treatment with the CD20 monospecific antibody, the cancer was refractory to said treatment.

28. The method in accordance with any one of embodiments 25 to 27, wherein said CD20 monospecific antibody was used in a combination treatment.

29. A method of treating relapsed/refractory large B-cell lymphoma in a human subject, the method comprising administering to said subject, a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein the subject achieves a complete response and a minimal residual disease (MRD) negativity.

30. The method in accordance with embodiment 29, wherein the subject is in complete response and is minimal residual disease (MRD) negative, when the antibody has been administered for 6 months or more, such as when the antibody has been administered for 7 months or more, 8 months or more, 9 months or more, 10 months or more, 11 months or more or such as for 12 months or more.

31. The method in accordance with embodiment 29 or 30, wherein when the antibody has been administered to the subject for 9 months:
the probability of progression free survival of said subject is 60% or more, such as 65% or more 70% or more, 75% or more, or such as 80% or more; and the subject is MRD negative.

32. The method in accordance with any one of embodiments 29 to 31, wherein the subject has primary refractory disease and achieves a complete response and a minimal residual disease (MRD) negativity.

33. The method in accordance with any one of embodiments 29 to 31, wherein the subject is CAR T-naïve and achieves a complete response and a minimal residual disease (MRD) negativity.

34. The method in accordance with any one of embodiments 29 to 31, wherein the subject has primary refractory disease and achieves a complete response and a minimal residual disease (MRD) negativity.

35. The method in accordance with any one of embodiments 29 to 31, wherein the subject is refractory to prior Car T treatment and achieves a complete response and a minimal residual disease (MRD) negativity.

36. The method in accordance with any one of embodiments 29 to 31, wherein the subject has DH/TH rearrangements and achieves a complete response and a minimal residual disease (MRD) negativity.

37. The method in accordance with any one of the preceding embodiments, wherein the patient received further prior lines of treatment for said B-cell NHL.

38. The method in accordance with any one of the preceding embodiments, wherein said bispecific antibody is administered at a dose of at least 40 mg.

39. The method in accordance with any one of the preceding embodiments, wherein said bispecific antibody is administered at a dose in the range of between 40 mg to 100 mg.

40. The method in accordance with any one of the preceding embodiments, wherein said bispecific antibody is administered at a dose of at least 48 mg.

41. The method in accordance with any one of the preceding embodiments, wherein said bispecific antibody is administered at a dose of at least 60 mg.

42. The method in accordance with any one of the preceding embodiments, wherein said bispecific antibody is administered at a dose of 48 mg.

43. The method in accordance with any one of the preceding embodiments, wherein said bispecific antibody is administered at a dose of 60 mg.

44. The method in accordance with any one of the preceding embodiments, wherein said dose is administered weekly.

45. The method in accordance with embodiment 44, wherein said weekly administration is performed at least 4 times.

46. The method in accordance with embodiment 44 or 45, wherein after said weekly administration, said antibody is administered once every two weeks.

47. The method in accordance with embodiment 46, wherein said biweekly administration is performed (at least) 6 times.

48. The method in accordance with embodiment 46 or 47, wherein after said administration once every two weeks, said antibody is administered once every four weeks.

49. The method in accordance with any one of embodiments 44 to 48, wherein prior to administering said weekly dose, a priming dose of said bispecific antibody is administered.

50. The method in accordance with embodiment 49, wherein said priming dose is administered one week prior to administering the first dose of said weekly dose.

51. The method in accordance with embodiment 49 or 50, wherein said priming dose is in the range of 50-1000 μg.

52. The method in accordance with any one of embodiments 49 to 51, wherein said priming dose is 160 μg.

53. The method in accordance with any one of embodiments 49 to 51, wherein said priming dose is 800 μg.

54. The method in accordance with any one of embodiments 49 to 53 wherein after administering said priming dose and prior to administering said weekly dose, an intermediate dose of said bispecific antibody is administered.

55. The method in accordance with embodiment 54, wherein said priming dose is administered two weeks, and said intermediate dose is administered one week, before administering the first dose of said weekly dose.

56. The method in accordance with any one of embodiments 54 or 55, wherein said intermediate dose is in the range of 600-5000 μg, such as 1200 μg.

57. The method in accordance with any one of embodiments 54 to 56, wherein said intermediate dose is 800 μg.

58. The method in accordance with any one of embodiments 54 to 56, wherein said intermediate dose is 4000 μg.

59. The method in accordance with any one of embodiments 29 to 58, wherein the bispecific antibody is administered subcutaneously to the subject in 28-day cycles.

60. The method in accordance with any one of embodiments 54 to 59, wherein the method of treatment comprises administering the bispecific antibody subcutaneously in 28-day cycles, wherein on:
a) Day 1, 8, 15 and 22 of the first cycle, a priming dose is administered at day 1, an intermediate dose at day 8, and a dose of 48 mg at days 15, 22; for the first cycle; on
b) Day 1, 8, 15 and 22 of cycles 2-3, a dose of 48 mg is administered; on
c) Day 1, 15 of cycles 4-9, a dose of 48 mg is administered; and on
d) Day 1, of further subsequent cycles, a dose of 48 mg is administered.

61. The method in accordance with embodiment 54 to 59, wherein the method of treatment comprises administering the bispecific antibody subcutaneously in 28-day cycles, wherein on:
a) Day 1, 8, 15 and 22 the first cycle, a priming dose of 160 μg is administered at day 1, an intermediate dose of 800 μg at day 8, and a dose of 48 mg at days 15, 22; for the first cycle; on
b) Day 1, 8, 15 and 22 of cycles 2-3, a dose of 48 mg is administered;
c) Day 1, 15 of cycles 4-9, a dose of 48 mg is administered; and on
d) Day 1, of further subsequent cycles, a dose of 48 mg is administered.

62. The method in accordance with any one of embodiments 54 to 59, wherein the method of treatment comprises administering the bispecific antibody subcutaneously in 28-day cycles, wherein on:
a) Day 1, 8, 15 and 22 the first cycle, a priming dose is administered at day 1, an intermediate dose at day 8, and a dose of 60 mg at days 15, 22; for the first cycle; on
b) Day 1, 8, 15 and 22 of cycles 2-3, a dose of 60 mg is administered; on
c) Day 1, 15 of cycles 4-9, a dose of 60 mg is administered; and on
d) Day 1, of further subsequent cycles, a dose of 60 mg is administered.

63. The method in accordance with any one of embodiments 54 to 57, wherein the method of treatment comprises administering the bispecific antibody subcutaneously in 28-day cycles, wherein on:
a) Day 1, 8, 15 and 22 the first cycle, a priming dose of 160 μg is administered at day 1, an intermediate dose of 800 μg at day 8, and a dose of 60 mg at days 15, 22; for the first cycle; on
b) Day 1, 8, 15 and 22 of cycles 2-3, a dose of 60 mg is administered;
c) Day 1, 15 of cycles 4-9, a dose of 60 mg is administered;
d) Day 1, of further subsequent cycles, a dose of 60 mg is administered.

64. The method in accordance with any one of embodiments 60 to 63, wherein said B-cell NHL is FL, DLBCL and/or HGBCL.

65. The method in accordance with any one of the preceding embodiments, wherein said subject has manageable cytokine release syndrome (CRS) of grade 1 or grade 2 during treatment.

66. The method in accordance with any one of the preceding embodiments, wherein said subject does not experience tumor lysis syndrome.

67. The method in accordance with any one of the preceding embodiments, wherein said subject is treated with prophylaxis for cytokine release syndrome (CRS).

68. The method in accordance with embodiment 67, wherein said prophylaxis includes the administration of a corticosteroid.

69. The method in accordance with embodiment 67 or 68, wherein said prophylaxis is administered at the same day as the bispecific antibody.

70. The method in accordance with any one of embodiment 67 to 69, wherein said prophylaxis is administered at subsequent days 2-3, and optionally day 4, or at subsequent days 2-4.

71. The method in accordance with any one of embodiments 67 to 70, wherein when said prophylaxis is administered at the same day as the bispecific antibody, said prophylaxis is administered 30-120 minutes prior to said administration of the bispecific antibody.

72. The method in accordance with any one of embodiments 68 to 71, wherein said corticosteroid is prednisolone, e.g. at an intravenous dose of 100 mg, or equivalent thereof, including oral dose.

73. The method in accordance with any one of the preceding embodiments, wherein said human subject is treated with premedication to reduce reactions to injections.

74. The method in accordance with embodiment 73, wherein said premedication includes the administration of antihistamines.

75. The method in accordance with embodiment 73 or 74, wherein said premedication includes the administration of antipyretics.

76. The method in accordance with any one of embodiments 74 or 75, wherein said antihistamine is diphenhydramine, e.g. at an intravenous or oral dose 50 mg, or equivalent thereof.

77. The method in accordance with any one of embodiments 75 or 76, wherein said antipyretic is acetaminophen, e.g. at an oral dose of 650-1000 mg, or equivalent thereof.

78. The method in accordance with any one of embodiment 73 to 77, wherein said premedication is administered at the same day as the bispecific antibody.

79. The method in accordance with embodiment 73, wherein said premedication administered 30-120 minutes prior to said administration of the bispecific antibody.

80. The method in accordance with any one of the preceding embodiments, wherein said prophylaxis as defined in embodiments 67 to 80 is administered during the first cycle.

81. The method in accordance with any one of the preceding embodiments, wherein said premedication as defined in embodiments 73 to 80, is administered during the first cycle.

82. The method in accordance with embodiment 80 or 81, wherein said prophylaxis is administered during the second cycle when the human subject experiences CRS greater than grade 1 after the fourth administration of the bispecific antibody in cycle 1.

83. The method in accordance with any one of embodiments 80 to 82, wherein said prophylaxis is continued during a subsequent cycle, when in the last administration of the bispecific antibody of the previous cycle, the human subject experiences CRS greater than grade 1.

84. The method in accordance with any one of embodiments 73 to 83, wherein said premedication is optionally administered during the second cycle.

85. The method in accordance with embodiment 84, wherein said premedication is optionally administered during subsequent cycles.

86. The method in accordance with any one of the preceding embodiments, wherein the bispecific antibody comprises:
i. a first binding arm comprising a first antigen-binding region binding to human CD3ε (epsilon), wherein said first antigen-binding region comprises a heavy chain variable (VH) region CDR1, CDR2, and CDR3 having the sequences as set forth in SEQ ID NOS: 1, 2, and 3, respectively, and a light chain variable (VL) region CDR1, CDR2, and CDR3 having the sequences as set forth in SEQ ID NO: 4, the sequence GTN, and the sequence as set forth in SEQ ID NO: 5, respectively, and
ii. a second binding arm comprising a second antigen-binding region binding to human CD20, wherein said second antigen-binding region comprises a heavy chain variable (VH) region CDR1, CDR2, and CDR3 having the sequences as set forth in SEQ ID NOS: 8, 9, and 10, respectively, and a light chain variable (VL) region CDR1, CDR2, and CDR3 having the sequences as set forth in SEQ ID NO: 11, the sequence DAS, and the sequence as set forth in SEQ ID NO: 12, respectively.

87. The method in accordance with any one of the preceding embodiments, wherein the bispecific antibody comprises:
i. a first binding arm comprising a first antigen-binding region binding to human CD3ε (epsilon), comprising a variable heavy chain region and a variable light chain region wherein the variable heavy chain region comprises SEQ ID NO: 6; and wherein the variable light chain region comprises SEQ ID NO: 7; and
ii. a second binding arm comprising a second antigen-binding region binding to human CD20, comprising a variable heavy chain region and a variable light chain region wherein the variable heavy chain region comprises SEQ ID NO: 13; and wherein the variable light chain region comprises SEQ ID NO: 14.

88. The method in accordance with any one of the preceding embodiments, wherein the first binding arm of the bispecific antibody is derived from a humanized antibody, preferably from a full-length IgG1,λ (lambda) antibody.

89. The method in accordance with embodiment 88, wherein said bispecific antibody comprises a λ light chain constant region as defined in SEQ ID NO: 22.

90. The method in accordance with any one of the preceding embodiments, wherein the second binding arm of the bispecific antibody is derived from a human antibody, preferably from a full-length IgG1,κ (kappa) antibody.

91. The method in accordance with embodiment 90, wherein said bispecific antibody comprises a k light chain constant region as defined in SEQ ID NO: 23.

92. The method in accordance with any one of the preceding embodiments, wherein the bispecific antibody is a full-length antibody with a human IgG1 constant region.

93. The method in accordance with any one of the preceding embodiments, in which the bispecific antibody comprises an inert Fc region.

94. The method in accordance with embodiment 93, wherein the bispecific antibody comprises in the first and second heavy chain at the positions corresponding to positions L234, L235, and D265 in the human IgG1 heavy chain of SEQ ID NO: 15 of both the first heavy chain and the second heavy chain, the amino acids F, E, and A, respectively.

95. The method in accordance with any one of the preceding embodiments, wherein the bispecific antibody comprises in said first heavy chain the amino acid L in the position corresponding to F405 in a human IgG1 heavy chain of SEQ ID NO: 15, and wherein said second heavy chain comprises the amino acid R in the position corresponding to K409 in a human IgG1 heavy chain of SEQ ID NO: 15 is R, or vice versa.

96. The method in accordance with any one of the preceding embodiments, wherein the bispecific antibody comprises in the first and second heavy chain at the positions corresponding to positions L234, L235, and D265 in the human IgG1 heavy chain of SEQ ID NO: 15 of both the first heavy chain and the second heavy chain, the amino acids F, E, and A, respectively, and wherein in said first heavy chain the amino acid L in the position corresponding to F405 in a human IgG1 heavy chain of SEQ ID NO: 15, and wherein said second heavy chain comprises the amino acid R in the position corresponding to K409 in a human IgG1 heavy chain of SEQ ID NO: 15 is R, or vice versa.

97. The method in accordance with embodiment 96, wherein the bispecific antibody comprises constant regions as defined in SEQ ID NOs: 19 and 20.

98. The method in accordance with any one of the preceding embodiments, wherein the bispecific antibody comprises a heavy chain and a light chain as defined in SEQ ID NOS: 24 and 25, respectively, and a heavy chain and a light chain as defined in SEQ ID NOS: 26 and 27.

99. The method in accordance with any one of the preceding embodiments, the bispecific antibody consists of a heavy chain and a light chain as defined in SEQ ID NOs: 24 and 25, respectively, and a heavy chain and a light chain as defined in SEQ ID NOs: 26 and 27.

100. The method in accordance with any one of the preceding embodiments, wherein the bispecific antibody is epcoritamab, or a biosimilar thereof.

101. A method of predicting a likelihood of relapse and/or disease progression in a subject having B-cell Non-Hodgkin Lymphoma (B-NHL), the method comprising: Measuring a minimal residual disease (MRD) status in the subject, wherein the subject receives or has received a bispecific antibody at a dose of at least 24 mg, said bispecific antibody being a full-length antibody, that comprises
i. a first binding arm comprising a first antigen-binding region binding to human CD3ε (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein a positive MRD status is indicative of a likelihood of relapse and/or disease progression.

102. The method in accordance with embodiment 101, wherein the B-NHL is as defined in any one of clams 12 to 18.

103. The method in accordance with embodiment 101 or 102, wherein the subject has received prior treatment as defined in any one of embodiments 19 to 28 and 37.

104. The method in accordance with any one of embodiments 101 to 103, wherein the bispecific antibody is administered as defined in any one of embodiments 37 to 63.

105. The method in accordance with any one of embodiments 101 to 104, wherein the subject is treated with prophylaxis for cytokine release syndrome (CRS) as defined in any one of embodiments 67 to 72 and 82 to 83.

106. The method in accordance with any one of embodiments 101 to 105, wherein the subject is treated with premedication to reduce reactions to injections as defined in any one of embodiments 73 to 81 and 84 to 85.

107. The method in accordance with any one of embodiments 101 to 106, wherein the bispecific antibody is as defined in any one of embodiments 86 to 100.

### Sequences

TABLE 1
Bold and underlined are FE; A; L and R, corresponding with positions 234 and 235;
265; 405 and 409, respectively, said positions being in accordance with EU-numbering. In
variable regions, said CDR regions that were annotated in accordance with IMGT definitions
are underlined.

| SEQ ID NO: | Clone name | Sequence |
|---|---|---|
| SEQ ID NO: 1 | huCD3 VH CDR1 | GFTFNTYA |
| SEQ ID NO: 2 | huCD3 VH CDR2 | IRSKYNNYAT |
| SEQ ID NO: 3 | huCD3 VH CDR3 | VRHGNFGNSYVSWFAY |
| SEQ ID NO: 4 | huCD3 VL CDR1 | TGAVTTSNY |
| | huCD3 VL CDR2 | GTN |
| SEQ ID NO: 5 | huCD3 VL CDR3 | ALWYSNLWV |
| SEQ ID NO: 6 | huCD3 VH1 | EVKLVESGGGLVQPGGSLRLSCAASGFTFNTYAMNWVRQA |
| | | PGKGLEWVARIRSKYNNYATYYADSVKDRFTISRDDSKSSL |
| | | YLQMNNLKTEDTAMYYCVRHGNFGNSYVSWFAYWGQGTL |
| | | VTVSS |
| SEQ ID NO: 7 | huCD3 VL1 | QAVVTQEPSFSVSPGGTVTLTCRSSTGAVTTSNYANWVQQT |
| | | PGQAFRGLIGGTNKRAPGVPARFSGSLIGDKAALTITGAQA |
| | | DDESIYFCALWYSNLWVFGGGTKLTVL |
| SEQ ID NO: 8 | VH CD20-7D8 | GFTFHDYA |
| | CDR1 | |
| SEQ ID NO: 9 | VH CD20-7D8 | ISWNSGTI |
| | CDR2 | |
| SEQ ID NO: 10 | VH CD20-7D8 | AKDIQYGNYYYGMDV |
| | CDR3 | |
| SEQ ID NO: 11 | VL CD20-7D8 | QSVSSY |
| | CDR1 | |
| | VL CD20-7D8 | DAS |
| | CDR2 | |
| SEQ ID NO: 12 | VL CD20-7D8 | QQRSNWPIT |
| | CDR3 | |
| SEQ ID NO: 13 | VH CD20-7D8 | EVQLVESGGGLVQPDRSLRLSCAASGFTFHDYAMHWVRQA |
| | | PGKGLEWVSTISWNSGTIGYADSVKGRFTISRDNAKNSLYL |
| | | QMNSLRAEDTALYYCAKDIQYGNYYYGMDVWGQGTTVTVS |
| | | S |
| SEQ ID NO: 14 | VL CD20-7D8 | EIVLTQSPATLSLSPGERATLSCRASQSVSSYLAWYQQKPGQ |
| | | APRLLIYDASNRATGIPARFSGSGSGTDFTLTISSLEPEDFA |
| | | VYYCQQRSNWPITFGQGTRLEIK |
| SEQ ID NO: 15 | IgG1 heavy chain | ASTKGPSVFPLAPSSKSTSGGTAALGCLVKDYFPEPVTVSW |
| | constant region- | NSGALTSGVHTFPAVLQSSGLYSLSSVVTVPSSSLGTQTYI |
| | WT | CNVNHKPSNTKVDKRVEPKSCDKTHTCPPCPAPELLGGPSV |
| | (amino acids | FLFPPKPKDTLMISRTPEVTCVVVDVSHEDPEVKFNWYVDG |
| | positions 118-447 | VEVHNAKTKPREEQYNSTYRVVSVLTVLHQDWLNGKEYKCK |
| | according to EU | VSNKALPAPIEKTISKAKGQPREPQVYTLPPSREEMTKNQV |
| | numbering). | SLTCLVKGFYPSDIAVEWESNGQPENNYKTTPPVLDSDGSF |
| | CH3 region italics | FLYSKLTVDKSRWQQGNVFSCSVMHEALHNHYTQKSLSLSP |
| | | G |
| SEQ ID NO: 16 | IgG1-LFLEDA | ASTKGPSVFPLAPSSKSTSGGTAALGCLVKDYFPEPVTVSW |
| | heavy chain | NSGALTSGVHTFPAVLQSSGLYSLSSVVTVPSSSLGTQTYIC |
| | constant region | NVNHKPSNTKVDKRVEPKSCDKTHTCPPCPAPEFEGGPSVF |
| | (amino acids | LFPPKPKDTLMISRTPEVTCVVVAVSHEDPEVKFNWYVDGV |
| | positions 118-447 | EVHNAKTKPREEQYNSTYRVVSVLTVLHQDWLNGKEYKCK |
| | according to EU | VSNKALPAPIEKTISKAKGQPREPQVYTLPPSREEMTKNQVS |
| | numbering). | LTCLVKGFYPSDIAVEWESNGQPENNYKTTPPVLDSDGSFFL |
| | | YSKLTVDKSRWQQGNVFSCSVMHEALHNHYTQKSLSLSPG |
| SEQ ID NO: 17 | IgG1 F405L | ASTKGPSVFPLAPSSKSTSGGTAALGCLVKDYFPEPVTVSW |
| | (amino acids | NSGALTSGVHTFPAVLQSSGLYSLSSVVTVPSSSLGTQTYIC |
| | positions 118-447 | NVNHKPSNTKVDKRVEPKSCDKTHTCPPCPAPELLGGPSVF |
| | according to EU | LFPPKPKDTLMISRTPEVTCVVVDVSHEDPEVKFNWYVDGV |
| | numbering) | EVHNAKTKPREEQYNSTYRVVSVLTVLHQDWLNGKEYKCK |
| | | VSNKALPAPIEKTISKAKGQPREPQVYTLPPSREEMTKNQVS |
| | | LTCLVKGFYPSDIAVEWESNGQPENNYKTTPPVLDSDGSFL |
| | | LYSKLTVDKSRWQQGNVFSCSVMHEALHNHYTQKSLSLSP |
| | | G |
| SEQ ID NO: 18 | IgG1-K409R | ASTKGPSVFPLAPSSKSTSGGTAALGCLVKDYFPEPVTVSW |
| | (amino acids | NSGALTSGVHTFPAVLQSSGLYSLSSVVTVPSSSLGTQTYIC |
| | positions 118-447 | NVNHKPSNTKVDKRVEPKSCDKTHTCPPCPAPELLGGPSVF |
| | according to EU | LFPPKPKDTLMISRTPEVTCVVVDVSHEDPEVKFNWYVDGV |
| | numbering) | EVHNAKTKPREEQYNSTYRVVSVLTVLHQDWLNGKEYKCK |
| | | VSNKALPAPIEKTISKAKGQPREPQVYTLPPSREEMTKNQVS |
| | | LTCLVKGFYPSDIAVEWESNGQPENNYKTTPPVLDSDGSFFL |
| | | YSRLTVDKSRWQQGNVFSCSVMHEALHNHYTQKSLSLSPG |
| SEQ ID NO: 19 | IgG1-LFLEDA- | ASTKGPSVFPLAPSSKSTSGGTAALGCLVKDYFPEPVTVSW |
| | F405L (FEAL) | NSGALTSGVHTFPAVLQSSGLYSLSSVVTVPSSSLGTQTYIC |
| | (amino acids | NVNHKPSNTKVDKRVEPKSCDKTHTCPPCPAPEFEGGPSVF |
| | positions 118-447 | LFPPKPKDTLMISRTPEVTCVVVAVSHEDPEVKFNWYVDGV |
| | according to EU | EVHNAKTKPREEQYNSTYRVVSVLTVLHQDWLNGKEYKCK |
| | numbering) | VSNKALPAPIEKTISKAKGQPREPQVYTLPPSREEMTKNQVS |
| | | LTCLVKGFYPSDIAVEWESNGQPENNYKTTPPVLDSDGSFL |
| | | LYSKLTVDKSRWQQGNVFSCSVMHEALHNHYTQKSLSLSP |
| | | G |
| SEQ ID NO: 20 | IgG1-LFLEDA- | ASTKGPSVFPLAPSSKSTSGGTAALGCLVKDYFPEPVTVSW |
| | K409R (FEAR) | NSGALTSGVHTFPAVLQSSGLYSLSSVVTVPSSSLGTQTYIC |
| | (amino acids | NVNHKPSNTKVDKRVEPKSCDKTHTCPPCPAPEFEGGPSVF |
| | positions 118-447 | LFPPKPKDTLMISRTPEVTCVVVAVSHEDPEVKFNWYVDGV |
| | according to EU | EVHNAKTKPREEQYNSTYRVVSVLTVLHQDWLNGKEYKCK |
| | numbering) | VSNKALPAPIEKTISKAKGQPREPQVYTLPPSREEMTKNQVS |
| | | LTCLVKGFYPSDIAVEWESNGQPENNYKTTPPVLDSDGSFFL |
| | | YSRLTVDKSRWQQGNVFSCSVMHEALHNHYTQKSLSLSPG |
| SEQ ID NO: 21 | IgG1 CH3 region | GQPREPQVYTLPPSREEMTKNQVSLTCLVKGFYPSDIAVEW |
| | | ESNGQPENNYKTTPPVLDSDGSFFLYSKLTVDKSRWQQGN |
| | | VFSCSVMHEALHNHYTQKSLSLSPG |
| SEQ ID NO: 22 | Constant region | GQPKAAPSVTLFPPSSEELQANKATLVCLISDFYPGAVTVAW |
| | human lambda LC | KADSSPVKAGVETTTPSKQSNNKYAASSYLSLTPEQWKSHR |
| | | SYSCQVTHEGSTVEKTVAPTECS |
| SEQ ID NO: 23 | Constant region | RTVAAPSVFIFPPSDEQLKSGTASVVCLLNNFYPREAKVQW |
| | human kappa LC | KVDNALQSGNSQESVTEQDSKDSTYSLSSTLTLSKADYEKH |
| | | KVYACEVTHQGLSSPVTKSFNRGEC |
| SEQ ID NO: 24 | huCD3-LFLEDA- | EVKLVESGGGLVQPGGSLRLSCAASGFTFNTYAMNWVRQA |
| | F405L (FEAL) | PGKGLEWVARIRSKYNNYATYYADSVKDRFTISRDDSKSSL |
| | heavy chain | YLQMNNLKTEDTAMYYCVRHGNFGNSYVSWFAYWGQGTL |
| | | VTVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKRVEPKSCDKTHTCPPCPAPEFEG |
| | | GPSVFLFPPKPKDTLMISRTPEVTCVVVAVSHEDPEVKFNWY |
| | | VDGVEVHNAKTKPREEQYNSTYRVVSVLTVLHQDWLNGKE |
| | | YKCKVSNKALPAPIEKTISKAKGQPREPQVYTLPPSREEMTK |
| | | NQVSLTCLVKGFYPSDIAVEWESNGQPENNYKTTPPVLDSD |
| | | GSFLLYSKLTVDKSRWQQGNVFSCSVMHEALHNHYTQKSL |
| | | SLSPG |
| SEQ ID NO: 25 | huCD3 VL+CL | QAVVTQEPSFSVSPGGTVTLTCRSSTGAVTTSNYANWVQQ |
| | light chain | TPGQAFRGLIGGTNKRAPGVPARFSGSLIGDKAALTITGAQA |
| | | DDESIYFCALWYSNLWVFGGGTKLTVLGQPKAAPSVTLFPP |
| | | SSEELQANKATLVCLISDFYPGAVTVAWKADSSPVKAGVET |
| | | TTPSKQSNNKYAASSYLSLTPEQWKSHRSYSCQVTHEGST |
| | | VEKTVAPTECS |
| SEQ ID NO: 26 | CD20-7D8- | EVQLVESGGGLVQPDRSLRLSCAASGFTFHDYAMHWVRQA |
| | LFLEDA-K409R | PGKGLEWVSTISWNSGTIGYADSVKGRFTISRDNAKNSLYL |
| | (FEAR) | QMNSLRAEDTALYYCAKDIQYGNYYYGMDVWGQGTTVTVS |
| | heavy chain | SASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKRVEPKSCDKTHTCPPCPAPEFEGGPS |
| | | VFLFPPKPKDTLMISRTPEVTCVVVAVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSREEMTKNQ |
| | | VSLTCLVKGFYPSDIAVEWESNGQPENNYKTTPPVLDSDGS |
| | | FFLYSRLTVDKSRWQQGNVFSCSVMHEALHNHYTQKSLSL |
| | | SPG |
| SEQ ID NO: 27 | CD20-7D8 | EIVLTQSPATLSLSPGERATLSCRASQSVSSYLAWYQQKPG |
| | VL+CL | QAPRLLIYDASNRATGIPARFSGSGSGTDFTLTISSLEPED |
| | light chain | FAVYYCQQRSNWPITFGQGTRLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| SEQ ID NO: 28 | Human CD3 | MQSGTHWRVLGLCLLSVGVWGQDGNEEMGGITQTPYKVSI |
| | (epsilon) | SGTTVILTCPQYPGSEILWQHNDKNIGGDEDDKNIGSDED |
| | | HLSLKEFSELEQSGYYVCYPRGSKPEDANFYLYLRARVCE |
| | | NCMEMDVMSVATIVIVDICITGGLLLLVYYWSKNRKAKAK |
| | | PVTRGAGAGGRQRGQNKERPPPVPNPDYEPIRKGQRDLYS |
| | | GLNQRRI |
| SEQ ID NO: 29 | Human CD20 | MTTPRNSVNGTFPAEPMKGPIAMQSGPKPLFRRMSSLVGPT |
| | | QSFFMRESKTLGAVQIMNGLFHIALGGLLMIPAGIYAPICV |
| | | TVWYPLWGGIMYIISGSLLAATEKNSRKCLVKGKMIMNSLS |
| | | LFAAISGMILSIMDILNIKISHFLKMESLNFIRAHTPYINI |
| | | YNCEPANPSEKNSPSTQYCYSIQSLFLGILSVMLIFAFFQE |
| | | LVIAGIVENEWKRTCSRPKSNIVLLSAEEKKEQTIEIKEEV |
| | | VGLTETSSQPKNEEDIEIIPIQEEEEEETETNFPEPPQDQE |
| | | SSPIENDSSP |

### EXAMPLES

### DuoBody-CD3×CD20

DuoBody-CD3×CD20 is a bsAb recognizing the T-cell antigen CD3 and the B-cell antigen CD20. DuoBody-CD3×CD20 triggers potent T-cell-mediated killing of CD20-expressing cells. DuoBody-CD3×CD20 has a regular IgG1 structure.

Two parental antibodies, IgG1-CD3-FEAL, a humanized IgG1X, CD3E-specific antibody having heavy and light chain sequences as listed in SEQ ID NOs. 24 and 25, respectively, and IgG1-CD20-FEAR, derived from human IgG1K CD20-specific antibody 7D8 having heavy and light chain sequences as listed in SEQ ID NOs. 26 and 27, respectively, were manufactured as separate biological intermediates. Each parental antibody contains one of the complementary mutations in the CH3 domain required for the generation of DuoBody molecules (F405L and K409R, respectively). The parental antibodies comprised three additional mutations in the Fc region (L234F, L235E and D265A; FEA). The parental antibodies were produced in mammalian Chinese hamster ovary (CHO) cell lines using standard suspension cell cultivation and purification technologies. DuoBody-CD3×CD20 was subsequently manufactured by a controlled Fab-arm exchange (cFAE) process (Labrijn et al. 2013, Labrijn et al. 2014, Gramer et al. 2013). The parental antibodies are mixed and subjected to controlled reducing conditions. This leads to separation of the parental antibodies that, under re-oxidation, re-assemble. This way, highly pure preparations of DuoBody-CD3×CD20 (˜ 93-95%) were obtained. After further polishing/purification, final product was obtained, close to 100% pure. The DuoBody-CD3×CD20 concentration was measured by absorbance at 280 nm, using the theoretical extinction coefficient 8=1.597 mL·mg⁻¹ cm⁻¹. The final product was stored at 4° C. The glycosylation profile of DuoBody-CD3×CD20 indicated that it predominantly contains core-fucosylated N linked bi-antennary glycans with varying degrees of terminal galactose. For the product produced, an international proprietary name was obtained, i.e. epcoritamab.

DuoBody-CD3×CD20 was prepared (5 mg/ml or 60 mg/mL) as a sterile clear colorless to slightly yellow solution supplied as concentrate for solution for subcutaneous (SC) injection. DuoBody-CD3×CD20 contains buffering and tonicifying agents. All excipients (Sodium acetate trihydrate, Acetic acid, Sodium hydroxide, Sorbitol, Polysorbate 80, Water for injection) and amounts thereof in the formulated product were pharmaceutically acceptable for subcutaneous injection products. Appropriate doses were reconstituted to a volume of about 1 mL for subcutaneous injection.

### Clinical Trials:

### GCT3013-01 (NCT03625037)

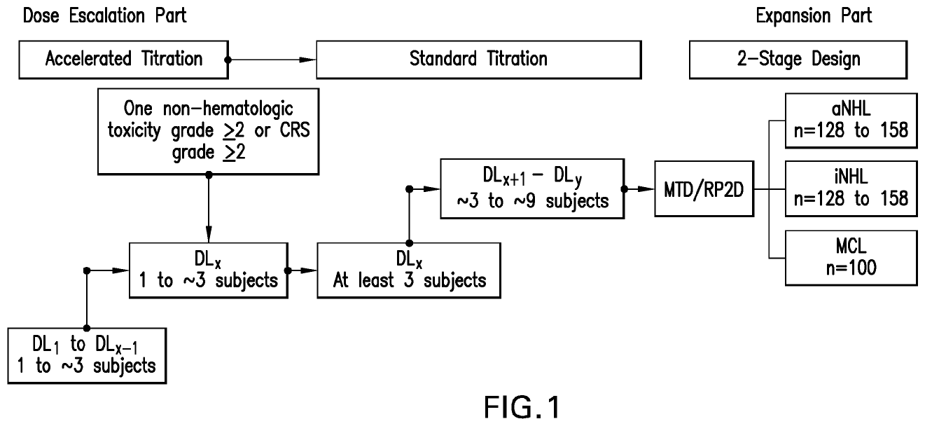

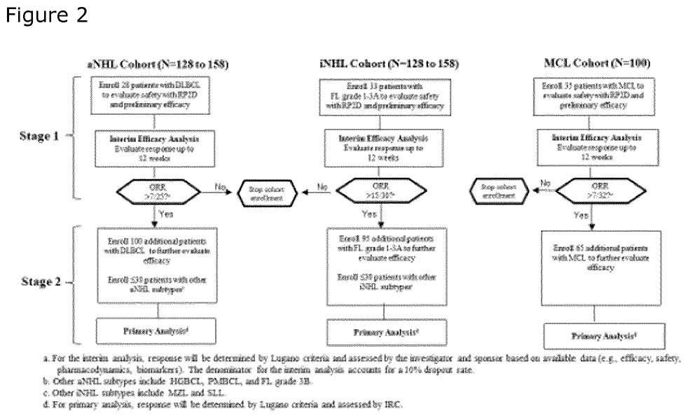

GCT3013-01 is an open-label, 2-part (dose escalation and expansion) trial in patients, human subjects, 18 years or older with a documented CD20+ mature B-cell neoplasm, and relapsed and/or refractory B-cell NHL. The trial design is provided in FIGS. 1 and 2.

### Dose Escalation

A fixed dose administration approach was selected to simplify dose administration because the number of T-cells, i.e. the effector cells for the function of GEN3013, were not expected to be correlated to weight and/or body surface area of the patients. The EMA Guideline on the Evaluation of Anti-cancer Medicinal Products in Man (EMA, 2012; EMA, 2017) acknowledges that the scientific support for body size-based dosing is weak and recommends modelling and simulation approaches using i.a. PK data to define a dosing strategy.

The purpose of the dose escalation part was to establish the maximum tolerated dose (MTD) and/or arrive at a dosing strategy for epcoritamab. Details on the design of the dose escalation part and PK/PD modelling are provided in WO 2021/224499 together with preliminary conclusions.

### Inclusion Criteria

Each potential patient must satisfy all of the following criteria to be enrolled in the dose escalation part of trial.
1. Patient must be 18 years of age or older.
2. Documented CD20+ mature B-cell neoplasm according to WHO classification (Swerdlow et al., 2016, Blood 127, 2375-2390.) or WHO classification 2008.
a. Diffuse large B-cell lymphoma-de novo or transformed.
b. High-grade B-cell lymphoma (Swerdlow et al., 2016).
c. Primary mediastinal large B-cell lymphoma.
d. Follicular lymphoma.
e. Mantle cell lymphoma.
f. Small lymphocytic lymphoma.
g. Marginal zone lymphoma (nodal, extranodal or mucosa associated).
3. Relapsed, progressive and/or refractory disease (Cheson et al., 2007, J Clin Oncol 25, 579-586) following treatment with an anti-CD20 monoclonal antibody (e.g., rituximab) potentially in combination with chemotherapy and/or relapsed after autologous stem cell rescue.
a. Patients must have exhausted or are ineligible for all standard therapeutic options.
b. Patients with indolent lymphoma (follicular, marginal zone or small lymphocytic lymphoma) must have a need for treatment initiation based on symptoms and/or disease burden.
4. Documentation of CD20+ mature B-cell neoplasm based on any representative pathology report.
5. At least 1 measurable site of disease based on computed tomography (CT) (or magnetic resonance imaging [MRI]) with involvement of 2 or more clearly demarcated lesions/nodes with a long axis >1.5 cm and short axis >1.0 cm or 1 clearly demarcated lesion/node with a long axis >2.0 cm and short axis ≥1.0 cm AND baseline FDG positron emission tomography (PET) scans must demonstrate positive lesion compatible with CT (or MRI) defined anatomical tumor sites.
6. ECOG performance status 0, 1 or 2.
7. Lymphocyte counts <5×109/L.
8. Platelet counts ≥75×109/L.
9. Hemoglobin level ≥9 g/dl (≥5.6 mmol/L) with or without transfusion.
10. Absolute neutrophil counts ≥1.0×109/L; growth factor support allowed in case of bone marrow involvement.
11. At least 4 weeks from last dose of unconjugated anti-CD20 targeting therapy till first dose of GEN3013.
12. At least 12 weeks from last dose of radio-conjugated or toxin-conjugated compound till first dose of GEN3013.
13. At least 4 weeks from last dose of investigational monoclonal antibodies, investigational chemotherapy, or other investigational anti-cancer agent till first dose of GEN3013.
14. Resolution of toxicities from prior therapy to a grade that does not contraindicate trial participation in the opinion of the investigator.
15. If receiving glucocorticoid treatment at screening, treatment must be tapered down and administered with a maximum of 25 mg daily in the last 14 days before the first dose of GEN3013.
16. Before the first dose of GEN3013, during the trial and for 12 months after last administration of GEN3013, a woman must be either
a. Not of childbearing potential *: premenarchal; postmenopausal (>45 years of age with amenorrhea for at least 12 months or any age with amenorrhea for at least 6 months and a serum follicle stimulating hormone [FSH] level >40 IU/L or mIU/mL); permanently sterilized (e.g., bilateral tubal occlusion [which includes tubal ligation procedures as consistent with local regulations], hysterectomy, bilateral salpingectomy, bilateral oophorectomy); or otherwise be incapable of pregnancy
b. Of childbearing potential and practicing a highly effective method of birth control (as defined by the EU Clinical Trial Facilitation Group) consistent with local regulations regarding the use of birth control methods for patients participating in clinical trials: e.g., established use of oral, injected or implanted combined (estradiol and progesterone containing) hormonal contraception; placement of an intrauterine device (IUD) or intrauterine system (IUS); male partner sterilization (the vasectomized partner should be the sole partner for that patient); true abstinence (when this is in line with the preferred and usual lifestyle of the patient).
   If the childbearing potential changes after start of the trial (e.g., woman who is not heterosexually active becomes active, premenarchal woman experiences menarche) a woman must begin a highly effective method of birth control, as described under 16b.
17. A man who is sexually active with a woman of childbearing potential must agree to use a barrier method of birth control (that is the use of condom) during the trial and for 12 months after receiving the last dose of GEN3013.
18. Women must agree not to donate eggs (ova, oocytes) for the purposes of assisted reproduction during the trial and for 12 months after receiving the last dose of GEN3013. Men must also not donate sperm during the trial and for 12 months after receiving the last dose of GEN3013.
19. The patient understands the purpose of the trial and procedures required for the trial and is capable of giving signed informed consent as which includes compliance with the requirements and restrictions listed in the informed consent form (ICF) and in this protocol.
20. Each patient must sign a separate ICF if he or she agrees to provide sample(s) for evaluation of DNA. Refusal to give consent for the optional DNA research samples does not exclude a patient from participation in the dose escalation part of the trial.

### Exclusion Criteria

Any potential patient who meets any of the following criteria will be excluded from participating in the dose escalation part of the trial:
1. Primary central nervous system (CNS) lymphoma or known CNS involvement by lymphoma at screening as confirmed by magnetic resonance imaging (MRI)/computed tomography (CT) scan (brain) and, if clinically indicated, by lumbar puncture.
2. Known past or current malignancy other than inclusion diagnosis, except for:
   a. Cervical carcinoma of Stage 1B or less.
   b. Non-invasive basal cell or squamous cell skin carcinoma.
   c. Non-invasive, superficial bladder cancer.
   d. Prostate cancer with a current PSA level <0.1 ng/ml. e. Any curable cancer with a complete response (CR) of >2 years duration
3. AST, and/or ALT >3× upper limit of normal.
4. Total bilirubin >1.5× upper limit of normal.
5. Creatinine clearance <45 mL/min (see Appendix 1).
6. Known clinically significant cardiac disease, including:
   Onset of unstable angina pectoris within 6 months of signing ICF
   Acute myocardial infarction within 6 months of signing ICF.
   Congestive heart failure (grade III or IV as classified by the New York Heart Association (see Appendix 2) and/or known decrease ejection fraction of <45%.
7. Chronic ongoing infectious diseases (except hepatitis B or hepatitis C) requiring treatment (excluding prophylactic treatment) at the time of enrolment or within the previous 2 weeks prior to the first dose of GEN3013.
8. Confirmed history or current autoimmune disease or other diseases resulting in permanent immunosuppression or requiring permanent immunosuppressive therapy. Low-dose prednisolone for rheumatoid arthritis or similar conditions is allowed.
9. Seizure disorder requiring therapy (such as steroids or anti-epileptics).
10. Any prior therapy with an investigational bispecific antibody targeting CD3 and CD20.
11. Prior treatment with chimeric antigen receptor T-cell (CAR-T) therapy within 30 days prior to first GEN3013 administration.
12. Eligible for curative intensive salvage therapy followed by high dose chemotherapy with HSCT rescue.
13. Autologous HSCT within 100 days prior to first GEN3013 administration, or any prior allogeneic HSCT or solid organ transplantation.
14. Active hepatitis B or hepatitis C. If laboratory evidence for a chronic infection with hepatitis B close monitoring and prophylactic therapy is required.
15. Known human immunodeficiency virus (HIV) infection.
16. Exposed to live or live attenuated vaccine within 4 weeks prior to signing ICF.
17. Pregnancy or breast feeding.
18. Patient has any condition for which, in the opinion of the investigator, participation would not be in the best interest of the patient (e.g., compromise the well-being) or that could prevent, limit, or confound the protocol-specified assessments.
19. Known hypersensitivity to allopurinol or rasburicase.

### Primary Endpoints

The primary endpoints of the dose escalation part are Dose-limiting toxicities (DLTs) and Adverse Effects (AEs). The evaluation of DLTs will be based on the Dose-Determining Set. The information from the DLTs will be used to guide the escalation and de-escalation rules as per the mBOIN design. See Appendix 9 for operational characteristics more details of the mBOIN design.

### Secondary Endpoints

1. Anti-lymphoma activity. Constitutional symptoms will be summarized and listed. Reduction in tumor size (based on percent change of SPD from baseline in CT scan or MRI evaluations) will be listed, summarized and presented in graphs, both over time and as best reduction per patient (waterfall plot). Objective and best response (ORR, PR and CR) will be classified according to the Recommendations for Initial Evaluation, Staging, and Response Assessment of Hodgkin and Non-Hodgkin Lymphoma: The Lugano Classification (Cheson et al., 2014 J Clin Oncol 32, 3059-3068.). Patients with partial response (PR) and complete response (CR) are considered as responders while all other categories, including “not evaluable”, are considered as non-responders. Patients with CR, PR or stable disease (SD) are considered to be in disease control. In the dose escalation part, response evaluation will be performed by the investigator.

2. Duration of response. DOR is defined as the time from the first documentation of response (CR or PR) to the date of PD or death, whichever occurs earlier. DOR will be analyzed using the same statistical methodology as PFS.

3. Progression-free survival. PFS is defined as the time from Day 1 of Cycle 1 to first documented PD or death due to any cause, whichever occurs earlier. PFS will be derived for all patients and presented graphically as well as summarized using survival analysis methods: distribution functions will be estimated using Kaplan-Meier technique. PFS will be censored at the date of the last disease assessment prior to start of subsequent anti-lymphoma therapy for patients who do not have disease progression and are alive at clinical cutoff. If there is no post-baseline tumor assessment for an alive patient, PFS will be censored on first dosing date.

Two definitions of PFS will be used in the analysis. Primary definition of PFS as being detailed in Table 2 will account for subsequent anti-lymphoma therapy and censor PFS at the last evaluable tumor assessment on or prior to the date of subsequent anti-lymphoma therapy. The subsequent anti-lymphoma therapies for PFS censoring in general consist of systemic anti-lymphoma therapy, and curative intent radiotherapy on one and only target lesion.

Secondary definition of PFS is irrespective of subsequent therapy and will not account for subsequent anti-lymphoma therapy. Censoring rules for the secondary definition of PFS is defined in Table 3.

Clinical progression without documented radiographical progression per Lugano or LYRIC criteria will not be considered progression for determination of PFS.

PFS rate at a landmark time T is defined as the probability that a subject has not progressed and is alive at time T following Day 1 of Cycle 1. PFS rates at 6, 9, and 12 months (or later times if available) will be presented along with 95% CI.

Main analysis of PFS will be based on IRC disease assessment per Lugano and LYRIC criteria using primary definition in the full analysis set (FAS).

Similar analyses based on investigator disease assessment in the FAS will also be conducted.

Analyses of PFS will also be performed based on the secondary definition of PFS.

TABLE 2
Censoring Scheme for Primary Definition of PFS

| | Date of Progression or | Censoring |
|---|---|---|
| Situation | Censoring | Rule |
| Incomplete or no baseline | Cycle 1 Day 1 (in lieu of | Censored |
| tumor assessments | randomization) | |
| Progression prior to | Date of PD | Progressed |
| subsequent anti-lymphoma | | |
| therapy | | |
| Death without subsequent | Date of death | Progressed |
| anti-lymphoma therapy | | |
| No progression or death | Date of last evaluable tumor | Censored |
| or new anti-lymphoma | assessment | |
| therapy | | |
| Subsequent anti-lymphoma | Date of last evaluable tumor | Censored |
| therapy started without PD | assessment before start of | |
| | subsequent anti-lymphoma | |
| | therapy | |

TABLE 3
Censoring Scheme for Secondary Definition of PFS

| | Date of Progression or | Censoring |
|---|---|---|
| Situation | Censoring | Rule |
| Incomplete or no baseline | Cycle 1 Day 1 (in lieu of | Censored |
| tumor assessments | randomization) | |
| Progression | Date of PD | Progressed |
| Death without progression | Date of death | Progressed |
| No progression or death | Date of last evaluable tumor | Censored |
| | assessment | |
| No post-baseline tumor | Cycle 1 Day 1 (in lieu of | Censored |
| assessment and no death | randomization) | |

4. Time to next anti-lymphoma therapy. Time to next anti-lymphoma therapy (TTNT) is defined as the time from Day 1 of Cycle 1 to first recorded administration of subsequent anti-lymphoma therapy or death due to any cause, whichever occurs earlier. TTNT will be analyzed using the same statistical methodology as PFS.

5. Overall survival. OS is defined as the time from Day 1 of Cycle 1 to death. OS will be analyzed using the same statistical methodology as PFS. If a patient is not known to have died, then OS will be censored at the latest date the patient was known to be alive.

### Expansion Part

### Inclusion Criteria:

Each potential patient had to satisfy all of the following criteria to be enrolled in the expansion part of trial.
1. Patient must be 18 years of age or older. Note: In countries where the legal age is 21 years of age; only patients 21 years of age or older are eligible.
2. Patient must meet the following entry criteria for the applicable expansion cohort:
   a. For expansion part R/R aNHL cohort:
      i. Documented CD20+ mature B-cell neoplasm according to WHO classification (Swerdlow et al., 2016) or WHO classification 2008 based on representative pathology report
         1. Diffuse large B-cell lymphoma (de novo or transformed from all indolent subtypes including Richter's transformation), including:
         b. Patients with “double-hit” or “triple-hit” DLBCL (technically classified in WHO 2016 as HGBCL, with MYC and BCL2 and/or BCL6 translocations) Note: Other double-/triple-hit lymphomas are not eligible
         2. Other aggressive B-NHL (beginning in Stage 2):
         a. Primary mediastinal (thymic) large B-cell lymphoma (PMBCL)
         b. High-grade B-cell lymphoma
         c. Follicular lymphoma grade 3B (FL 3B)
      ii. Relapsed or refractory disease and previously treated with at least 2 lines of systemic antineoplastic therapy including at least 1 anti-CD20 monoclonal antibody-containing therapy. Note: Relapsed disease is defined as disease that has recurred ≥6 months after completion of therapy. Refractory disease is defined as disease that either progressed during therapy or progressed within 6 months (<6 months) of completion of therapy.
      iii. Either failed prior autologous hematopoietic stem cell transplantation (HSCT), or ineligible for autologous HSCT due to age, ECOG performance status, comorbidities, and/or insufficient response to prior treatment
   b. For expansion part R/R iNHL cohort:
      i. Documented CD20+ mature B-cell neoplasm according to WHO classification (Swerdlow et al., 2016) or WHO classification 2008 based on representative pathology report
         1. Histologic confirmed FL grade 1, 2, or 3A at initial diagnosis without clinical or pathological evidence of transformation
         2. Marginal zone lymphomas (nodal, extranodal, and splenic)
         3. Small lymphocytic lymphoma
      ii. Relapsed or refractory disease and previously treated with at least 2 lines of systemic antineoplastic therapy including at least 1 anti-CD20 monoclonal antibody-containing therapy Note: Relapsed disease is defined as disease that has recurred ≥6 months after completion of therapy. Refractory disease is defined as disease that either progressed during therapy or progressed within 6 months (<6 months) of completion of therapy.
      iii. Previously treated with an alkylating agent or lenalidomide
      iv. Relapsed or refractory to the last prior line therapy. Previous lymphoma therapy is defined as 1 of the following: At least 2 months of single-agent therapy, at least 2 consecutive cycles of combination therapy, autologous HSCT, immunomodulatory therapy, or radioimmunotherapy
   c. For expansion part R/R MCL cohort:
      i. Documented CD20+ MCL according to WHO classification Swerdlow et al., 2016 or WHO classification 2008 based on representative pathology report with either cyclin D1 overexpression or presence of the translocation t (11; 14).
      ii. Stage II-IV with a need for treatment.
      iii. Previously treated with at least 2 prior lines of systemic antineoplastic therapy including at least 1 prior anti-CD20 mAb-containing regimen.
      iv. Previously treated with a BTKi and either progressing (relapsed or refractory) or intolerant to BTKi
      v. Relapsed or refractory to the most recent line of therapy. Note: Relapsed disease is defined as disease that has recurred ≥6 months after completion of therapy. Refractory disease is defined as disease that either progressed during therapy or progressed within 6 months (<6 months) of completion of therapy.
      vi. If bridging therapy with BTKi is administered during the screening period, the patient must be able to undergo a repeat baseline PET-CT (and bone marrow aspirate/biopsy, if applicable) to assess baseline disease status prior to first administration of GEN3013 (if bridging therapy to the start of GEN3013 administration is more than 2 weeks).
3. Measurable disease:
   a. Fluorodeoxyglucose (FDG)-avid lymphomas: Measurable disease with computerized tomography (CT) (or magnetic resonance imaging [MRI]) scan with involvement of 2 or more clearly demarcated lesions/nodes with a long axis >1.5 cm and short axis >1.0 cm (or 1 clearly demarcated lesion/node with a long axis >2.0 cm and short axis ≥1.0 cm) AND FDG positron emission tomography (PET) scan that demonstrates positive lesion(s) compatible with CT (or MRI) defined anatomical tumor sites
   b. FDG-nonavid lymphomas: Measurable disease with CT (or MRI) scan with involvement of 2 or more clearly demarcated lesions/nodes with a long axis >1.5 cm and short axis >1.0 cm or 1 clearly demarcated lesion/node with a long axis >2.0 cm and short axis ≥1.0 cm.
4. ECOG performance status 0, 1, or 2
5. Lymphocyte counts <5×109/L. For MCL: <50×109/L
6. Platelet counts ≥75×109/L or, in the presence of bone marrow involvement or splenomegaly, ≥50×109/L
7. Absolute neutrophil counts ≥1.0×109/L; growth factor support allowed in case of bone marrow involvement
8. Patient must meet the following criteria regarding time since previous anti-neoplastic agent(s):
   a. At least 4 weeks from last dose of non-investigational systemic chemotherapy
   b. At least 4 weeks or 5 half-lives from last dose of other non-investigational antineoplastic agents, whichever is shorter (except any anti-CD20 mAb or BTKi)
   c. At least 5 half-lives from last dose of investigational agents except for prior chimeric antigen receptor T-cell (CAR-T) therapy from which 30 days must pass prior to first GEN3013 administration.
9. Resolution of toxicities from prior therapy to a grade that does not contraindicate trial participation in the opinion of the investigator
10. If receiving glucocorticoid treatment at screening, must be a maximum daily dose of prednisone 10 mg (or equivalent) and a total of no more than 140 mg over the last 14 days prior to the first dose of GEN3013
11. Before the first dose of GEN3013, during the trial and for 12 months after last administration of GEN3013, a woman must be either
   a. Not of childbearing potential *: premenarchal; postmenopausal (>45 years of age with amenorrhea for at least 12 months or any age with amenorrhea for at least 6 months and a serum follicle stimulating hormone [FSH] level >40 IU/L or mIU/mL); permanently sterilized (e.g., bilateral tubal occlusion [which includes tubal ligation procedures as consistent with local regulations], hysterectomy, bilateral salpingectomy, bilateral oophorectomy); or otherwise be incapable of pregnancy
   b. Of childbearing potential and practicing a highly effective method of birth control (as defined by the EU Clinical Trial Facilitation Group) consistent with local regulations regarding the use of birth control methods for patients participating in clinical trials: e.g., established use of oral, injected or implanted combined (estradiol and progesterone containing) hormonal contraception; placement of an intrauterine device (IUD) or intrauterine system (IUS); male partner sterilization (the vasectomized partner should be the sole partner for that patient); true abstinence (when this is in line with the preferred and usual lifestyle of the patient)
      If the childbearing potential changes after start of the trial (e.g., woman who is not heterosexually active becomes active, premenarchal woman experiences menarche) a woman must begin a highly effective method of birth control, as described under 31b.
12. A man who is sexually active with a woman of childbearing potential must agree to use a barrier method of birth control (that is the use of condom) during the trial and for 12 months after receiving the last dose of GEN3013.
13. Women must agree not to donate eggs (ova, oocytes) for the purposes of assisted reproduction during the trial and for 12 months after receiving the last dose of GEN3013. Men must also not donate sperm during the trial and for 12 months after receiving the last dose of GEN3013.
14. The patient understands the purpose of the trial and procedures required for the trial and is capable of giving signed informed consent as which includes compliance with the requirements and restrictions listed in the informed consent form (ICF) and in this protocol
15. The patient must consent to provide sample(s) for evaluation of DNA.

### Exclusion Criteria

### Expansion Exclusion Criteria

Any potential patient who meets any of the following criteria will be excluded from participating in the expansion part of the trial.
1. Primary central nervous system (CNS) lymphoma or CNS involvement by lymphoma at screening as confirmed by mandatory magnetic resonance imaging (MRI)/computed tomography (CT) scan (brain) and, if clinically indicated, by lumbar puncture.
2. Known past or current malignancy other than inclusion diagnosis, except for:
   a. Cervical carcinoma of Stage 1B or less.
   b. Non-invasive basal cell or squamous cell skin carcinoma.
   c. Non-invasive, superficial bladder cancer.
   d. Prostate cancer with a current PSA level <0.1 ng/ml.
   e. Any curable cancer with a complete response (CR) of >2 years duration.
3. AST, and/or ALT >3× upper limit of normal.
4. Total bilirubin >1.5× upper limit of normal, unless bilirubin rise is due to Gilbert's syndrome or of non-hepatic origin.
5. Estimated GFR <45 mL/min/1.73m2.
6. Known clinically significant cardiac disease, including:
   Onset of unstable angina pectoris within 6 months of signing ICF.
   Acute myocardial infarction within 6 months of signing ICF.
   Congestive heart failure (grade III or IV as classified by the New York Heart. Association and/or known decrease ejection fraction of <45%.

### Expansion Exclusion Criteria

Any potential patient who meets any of the following criteria will be excluded from participating in the expansion part of the trial.
1. Primary central nervous system (CNS) lymphoma or CNS involvement by lymphoma at screening as confirmed by mandatory magnetic resonance imaging (MRI)/computed tomography (CT) scan (brain) and, if clinically indicated, by lumbar puncture.
2. Known past or current malignancy other than inclusion diagnosis, except for:
   a. Cervical carcinoma of Stage 1B or less.
   b. Non-invasive basal cell or squamous cell skin carcinoma.
   c. Non-invasive, superficial bladder cancer.
   d. Prostate cancer with a current PSA level <0.1 ng/ml.
   e. Any curable cancer with a complete response (CR) of >2 years duration
3. AST, and/or ALT >3× upper limit of normal
4. Total bilirubin >1.5× upper limit of normal, unless bilirubin rise is due to Gilbert's syndrome or of non-hepatic origin
5. Estimated GFR <45 mL/min/1.73m2 (see Appendix 1)
6. Known clinically significant cardiac disease, including:
   Onset of unstable angina pectoris within 6 months of signing ICF
   Acute myocardial infarction within 6 months of signing ICF.
   Congestive heart failure (grade III or IV as classified by the New York Heart Association (see Appendix 2) and/or known decrease ejection fraction of <45%

### GEN3013 Product Information

GEN3013 is supplied at 5 mg/mL or 60 mg/mL in 30 mM sodium acetate, 150 mM sorbitol, and 0.04% polysorbate, with a pH of 5.5.

GEN3013 is to be administered by SC injection. Injection site is preferably in the lower part of abdomen or the thigh. Change of injection site from left or right side or vice versa is recommended especially in the weekly administration part.

### Endpoints

### Primary Endpoints

The primary endpoint of the expansion part is ORR determined by the Recommendations for Initial Evaluation, Staging, and Response Assessment of Hodgkin and Non-Hodgkin Lymphoma: The Lugano Classification (Cheson et al., 2014). The interim analysis for Stage 1 for each cohort will be based on assessment by the investigator. The primary analysis will be conducted approximately 9 months after the last patient's first dose for the iNHL and aNHL cohorts, and approximately 6 months after the last patient's first dose for the MCL cohort; analysis will be based on assessment by IRC. Analysis of ORR as assessed by investigator will be included as a sensitivity analysis for the primary analysis. The final analysis will be conducted at the end of the trial per protocol. The DLBCL group, overall aNHL population, FL 1-3A group, and overall iNHL population, and the MCL cohort will be analyzed. Other sensitivity analyses will be specified in the Statistical Analysis Plan.

Patients with partial response (PR) and complete response (CR) are considered as responders while all other categories, including “not evaluable”, are considered as non-responders. Patients with CR, PR or stable disease (SD) are considered to be in disease control.

### Secondary Efficacy Endpoints

For DOR, PFS, TTR, DoCR and CR rate, the primary analysis will be based on assessment by IRC, and sensitivity analyses will be based on investigator assessment.
1. Anti-lymphoma activity. See Section 1 in re. dose escalation above for details on endpoint definition and response. In addition to Lugano criteria, response assessment in the expansion part will be determined according to LYRIC criteria (Cheson et al., 2016).
2. Duration of response (DoR). See Section 2 above in re. dose escalation for details on endpoint definition and analysis. In the expansion part, analysis will be based on response assessment by Lugano and LYRIC criteria, respectively.
3. Complete response rate (CR). CR rate is defined as the proportion of patients with CR. Analysis will be based on response assessment by Lugano and LYRIC criteria, respectively.
4. Duration of complete response (DoCR). DoCR is defined as the time from the first documentation of CR to the date of PD or death, whichever occurs earlier. DoCR will be analyzed using the same statistical methodology as PFS. The analysis will be based on response assessment by Lugano and LYRIC criteria, respectively.
5. Progression-free survival. See Section 3 above in re. dose escalation for details on endpoint definition and analysis. In the expansion part, analysis will be based on response assessment by Lugano and LYRIC criteria, respectively.
6. Overall survival. See Section 5 in re dose escalation for details on endpoint definition and analysis.
7. Time to response (TTR). TTR is defined as the time from Day 1 of Cycle 1 to first documentation of objective tumor response (PR or better). It will be derived for all patients achieving PR or CR. Analysis will be based on response assessment by Lugano and LYRIC criteria, respectively.
8. Time to next anti-lymphoma therapy. Please see Section 4 under dose escalation for details on endpoint definition and analysis.
9. Minimal residual disease. The rate of MRD negativity (MRD−) is defined as the proportion of patients with at least one MRD-sample.

MRD will be assessed using a ctDNA next generation sequencing assay (NNGS) (ClonoSEQ™ assay (Adaptive Biotechnologies, Seattle, WA, USA) determining the presence of DNA that encodes the B-cell receptor (BCR) expressed specifically by the cancer cells. After start of treatment, blood samples are requested at fixed timepoints to assess whether the amount of cancer DNA is declining, as a potential measure of (early) response, and to assess MRD at time of Positron emission tomography-computed tomography PET/CT response assessments. When a patient reaches a CR, MRD will be measured in blood to assess whether the cancer is still detectable or not. Additionally, as exploratory analysis, patients having bone marrow involvement documented at screening will have a portion of the aspirate collected to confirm CR used to assess MRD. Duration of MRD− is defined as the number of days from the first documentation of MRD-to the date of MRD status change (not MRD−). This will be analyzed using the same statistical methodology as PFS.

### Status of Trials

In total, approximately 569 subjects have been treated with epcoritamab monotherapy across 3 ongoing trials of in B-NHL: GCT3013-01 (n=359 subjects total; 68 subjects in the dose escalation part, 291 subjects in the expansion part), GCT3013-04 (n=66 subjects; 9 subjects in the dose escalation part, 57 subjects in the expansion part), and GCT3013-05 (n=174 subjects, approximately 87 treated with epcoritamab in this 1:1 randomized study).

Following up dose escalation, in the expansion part patients received treatment with epcoritamab subcutaneously in 28-day cycles, within cycle 1, day 1, 0.16 mg, day 8 0.8 mg, days 15 and 22, 48 mg; cycles 2-3, day 1, 8, 15 and 22, 48 mg; cycles 4-9, day 1, 15, 48 mg; and cycles 10+, 48 mg on day 1. 359 subjects have been enrolled in this trial.

TABLE 4
Doses by Cohort - GCT3013-01 Dose Escalation Part

| | | Number of |
|---|---|---|
| | Cycle 1 Doses (Days 1, 8, | Subjects |
| Cohort | 15, and 22) | (N = 68) |
| 1 | 0.004 mg, 0.0128 mg, 0.0128 | 1 |
| | mg, 0.0128 mg | |
| 2 ^{a} | 0.0128 mg, 0.04 mg, 0.04 mg, | 2 |
| | 0.04 mg | |
| 3 | 0.04 mg, 0.12 mg, 0.12 mg, 0.12 | 4 |
| | mg | |
| 3 ^{b} | 0.04 mg, 0.38 mg, 0.38 mg, 0.38 | 1 |
| | mg | |
| 4 | 0.12 mg, 0.38 mg, 0.38 mg, 0.38 | 1 |
| | mg | |
| 5 | 0.04 mg, 0.76 mg, 0.76 mg, 0.76 | 7 |
| | mg | |
| 6 | 0.04 mg, 0.25 mg, 1.5 mg, 1.5 | 5 |
| | mg | |
| 7 | 0.04 mg, 0.5 mg, 3 mg, 3 mg | 6 |
| 8 | 0.04 mg, 0.5 mg, 6 mg, 6 mg | 7 |
| 8 ^{b} | 0.08 mg, 0.5 mg, 6 mg, 6 mg | 2 |
| 9 | 0.04 mg, 0.8 mg, 12 mg, 12 mg | 3 |
| 9 ^{b} | 0.08 mg, 1.6 mg, 12 mg, 12 mg | 4 |
| 10 | 0.04 mg, 0.8 mg, 24 mg, 24 mg | 6 |
| 10 ^{b} | 0.16 mg, 0.8 mg, 24 mg, 24 mg | 4 |
| 11 | 0.08 mg, 0.8 mg, 48 mg, 48 mg | 3 |
| 11 ^{b} | 0.16 mg, 0.8 mg, 48 mg, 48 mg | 9 |
| 12 | 0.16 mg, 0.8 mg, 60 mg, 60 mg | 3 |

Note:
To bridge the gap between priming and continuously escalating full doses, an intermediate dose of epcoritamab was addedprior to dosing at the 1.5 mg full dose level.
The last dose in Cycle 1 (i.e., full dose) is continued in Cycle 2 and onwards.
^{a} One subject in Cohort 2 received only the priming dose due to discontinuation (disease progression) from the trial prior tocompletion of the dose-limiting toxicity period (i.e., 28 days) and was replaced.
^{b} Cohort intended for parallel evaluation.

In WO 2021/224499 a full dose of 48 mg was recommended for both DLBCL/HGBCL and FL, and for B-NHL, after a total of 61 subjects had been dosed in dose escalation. The recommendation was based on clinical efficacy, safety data and PK/PD modelling. At the end of dose escalation a total of 68 subjects had been treated with epcoritamab in the GCT3013-01 dose escalation part, including 15 subjects (22.1%) who, at the time of data cutoff, were continuing to receive epcoritamab in the 3 to 60 mg dosing groups (2 subjects at 3 mg, 1 subject at 6 mg, 2 subjects at 12 mg, 4 subjects at 24 mg, 4 subjects at 48 mg, and 2 subjects at 60 mg). Of the 53 subjects who had discontinued treatment, 46 subjects discontinued due to disease progression. Of the 68 subjects treated in the GCT3013-01 dose escalation part, the median age was 67.5 years, and the majority of subjects (45, 66.2%) were male. The median time from lymphoma diagnosis to first dose was 29.7 months. The median time since the most recent recurrence, relapse, or progression was 1.6 months. Of the various B-NHL subtypes enrolled, 46 subjects (67.6%) had DLBCL, and 12 subjects (17.6%) had FL. Patients enrolled had the following disease type: Diffuse large B-cell lymphoma, i.e. de novo (28), transformed (17) or unknown (1); high-grade B-cell lymphoma (3), primary mediastinal large B-cell lymphoma (1), follicular lymphoma (12), mantle-cell lymphoma (4), small lymphocytic lymphoma (1) and marginal zone lymphoma (1). Patients receiving the full dose included diffuse large B-cell lymphoma, i.e. de novo (9), Primary mediastinal large B-cell lymphoma (1), Follicular lymphoma (1), and Mantle-cell lymphoma (1).

As of Jan. 31, 2022, 291 subjects have been treated with epcoritamab in the GCT3013-01 expansion part, including 157 subjects in the aggressive B-cell non-Hodgkin lymphoma (aNHL) cohort, 101 subjects in the indolent B-cell non-Hodgkin lymphoma (iNHL) cohort, and 33 subjects in the MCL cohort. Of the 24 subjects (28.2%) who discontinued treatment, 14 subjects discontinued due to disease progression. Of the 291 subjects treated in the GCT3013-01 expansion part, the median age was 68.0 years, and the majority of subjects (60, 70.6%) were male. The median time from lymphoma diagnosis to first dose was 24.9 months. The median time since the most recent refractory or relapse was 1.13 months. Of the various B-NHL subtypes enrolled, 71 (83.5%) subjects had DLBCL, and 13 (15.3%) subjects had FL. In the aggressive NHL cohort, most subjects had DLBCL, i.e. de novo (44), transformed (17), not applicable (5), unknown (3), missing (1); and one patient had high-grade B-cell lymphoma. Of the subjects in the indolent B-cell non-Hodgkin lymphoma cohort, 12 had follicular lymphoma grade 1-3A and one of unknown grade.

TABLE 5
Subject Disposition - GCT3013-01 Expansion Part

| Number (%) | Cohort | | | | | | | |
|---|---|---|---|---|---|---|---|---|
| of subjects^{a} | aNHL | | iNHL | | MCL | | Total | |
| Full Analysis set (FAS) | 157 | (100%) | 101 | (100%) | 33 | (100%) | 291 | (100%) |
| Safety set | 157 | (100%) | 101 | (100%) | 33 | (100%) | 291 | (100%) |
| Treated subjects | | | | | | | | |
| Ongoing trial treatment | 54 | (34.4%) | 69 | (68.3%) | 21 | (63.6%) | 144 | (49.5%) |
| Discontinued from trial | 103 | (65.6%) | 32 | (31.7%) | 12 | (36.4%) | 147 | (50.4%) |
| treatment | | | | | | | | |
| Progressive disease^{b} | 73 | (46.5%) | 20 | (19.8%) | 7 | (21.2% | 100 | (34.4%) |
| Adverse event | 9 | (5.7%) | 1 | (1.0%) | 1 | (3.0%) | 11 | (3.8%) |
| Withdrawal by subject | 1 | (0.6%) | 3 | (3.0%) | 1 | (3.0) | 5 | (1.7%) |
| Decision to proceed with | 5 | (3.2%) | 4 | (4.0%) | 1 | (3.0%) | 10 | (3.4%) |
| transplant | | | | | | | | |
| Death | 2 | (1.3%) | 2 | (2.0%) | 0 | | 4 | (1.4%) |
| Other | 2 | (1.3%) | 1 | (1.0%) | 1 | (3.0%) | 4 | (1.4%) |
| Discontinued from trial | 68 | (43.3%) | 13 | (12.9%) | 8 | (24.2%) | 89 | (30.6%) |
| Death | 51 | (32.5%) | 10 | (9.9%) | 5 | (15.2%) | 66 | (22.7%) |
| Lost to follow-up | 0 | | 0 | | 0 | | 0 | |
| Withdrawal by subject | 7 | (4.5%) | 0 | | 2 | (6.1%) | 9 | (3.1%) |
| Other | 10 | (6.4%) | 3 | (3.0%) | 1 | (3.0%) | 14 | (4.8%) |

Note:
Percentages calculated based on number of subjects in FAS.
aNHL = aggressive B-cell non-Hodgkin lymphoma; FAS = Full Analysis set; iNHL = indolent B-cell non-Hodgkin lymphoma.
^{a}FAS and safety set include all subjects exposed to epcoritamab.
^{b}Progressive disease includes both clinical progression and documented radiographic disease progression.

### Pharmacodynamics

Epcoritamab induced rapid and sustained depletion of circulating B-cells (in the subset of patients with detectable B-cells, which are absent in most subjects due to prior anti-CD20 therapy). B-cells were detected with an antibody against CD19. Subsequent dosing induced expansion of circulating T-cells from baseline and moderate elevations of circulating IFNγ, IL-6, and TNFα (by serum immunoassay) at doses greater than 12 mg. Transient decrease in peripheral CD4+ and CD8+ T-cells was observed within 6 hours of first SC dose, which is consistent with T-cell margination seen with other bispecifics. Importantly, subsequent dosing induced expansion of T-cells from baseline. Step-up dosing and SC administration of epcoritamab were implemented to mitigate CRS. SC administration of epcoritamab resulted in moderate IFNγ, IL-6, and TNFα elevations.

### Efficacy

In the dose escalation part, evaluations of response to treatment were based on investigator assessments according to Lugano criteria (Cheson et al., 2014). Using the Full Analysis set, 18/46 subjects (39.1%) with DLBCL achieved a response (subjects with partial response or complete response are considered as responders). In subjects with FL, 9/12 (75.0%) achieved a response (Table 6).

TABLE 6
Best Overall Response by Disease, Lugano Classification by Investigator
Assessment - GCT3013-01 Dose Escalation Part - DLBCL and FL Subjects

| | Full | | | | | | | | |
|---|---|---|---|---|---|---|---|---|---|
| | Dose | | | | | | | | |
| Number (%) of Subjects | <0.76 | ≥0.76 | <12 | ≥12 | 12 | 24 | 48 | 48-60 | Total |
| | mg | mg | mg | mg | mg | mg | mg | mg | |
| Diffuse large B-cell lymphoma | N = 7 | N = 39 | N = 23 | N = 23 | N = 5 | N = 6 | N = 9 | N = 12 | N = 46 |
| Best overall response | | | | | | | | | |
| Complete response (CR) | 1 | 12 | 3 | 10 | 3 | 1 | 3 | 6 | 13 |
| | (14.3%) | (30.8%) | (13.0%) | (43.5%) | (60.0%) | (16.7%) | (33.3%) | (50.0%) | (28.3%) |
| Partial response (PR) | 0 | 5 | 0 | 5 | 1 | 0 | 4 | 4 | 5 |
| | | (12.8%) | | (21.7%) | (20.0%) | | (44.4%) | (33.3%) | (10.9%) |
| Stable disease (SD) | 0 | 4 | 3 | 1 | 0 | 1 | 0 | 0 | 4 |
| | 0 | (10.3%) | (13.0%) | (4.3%) | | (16.7%) | | | (8.7%) |
| Progressive disease | 6 | 16 | 17 | 5 | 1 | 4 | 0 | 0 | 22 |
| | (85.7%) | (41.0%) | (73.9%) | (21.7%) | (20.0%) | (66.7%) | | | (47.8%) |
| Not evaluable | 0 | 0 | 0 | 0 | 0 | 0 | 0 | 0 | 0 |
| No assessments^{a} | 0 | 2 | 0 | 2 | 0 | 0 | 2 | 2 | 2 |
| | | (5.1%) | | (8.7%) | | | (22.2%) | (16.7%) | (4.3%) |
| Overall response rate | 1 | 17 | 3 | 15 | 4 | 1 | 7 | 10 | 18 |
| (CR + PR) | (14.3%) | (43.6%) | (13.0%) | (65.2%) | (80.0%) | (16.7%) | (77.8%) | (83.3%) | (39.1%) |
| (95% CI)^{b} | (0.4%, | (27.8%, | (2.8%, | (42.7%, | (28.4%, | (0.4%, | (40.0%, | (51.6%, | (25.1%, |
| | 57.9%) | 60.4%) | 33.6%) | 83.6%) | 99.5%) | 64.1%) | 97.2%) | 97.9%) | 54.6%) |
| Complete response rate | 1 | 12 | 3 | 10 | 3 | 1 | 3 | 6 | 13 |
| (95% CI)^{b} | (14.3%) | (30.8%) | (13.0%) | (43.5%) | (60.0%) | (16.7%) | (33.3%) | (50.0%) | (28.3%) |
| | (0.4%, | (17.0%, | (2.8%, | (23.2%, | (14.7%, | (0.4%, | (7.5%, | (21.1%, | (16.0%, |
| | 57.9%) | 47.6%) | 33.6%) | 65.5%) | 94.7%) | 64.1%) | 70.1%) | 78.9%) | 43.5%) |
| Disease control rate (CR + PR + | 1 | 21 | 6 | 16 | 4 | 2 | 7 | 10 | 22 |
| SD | (14.3%) | (53.8%) | (26.1%) | (69.6%) | (80.0%) | (33.3%) | (77.8%) | (83.3%) | (47.8%) |
| (95% CI)^{b} | (0.4%, | (37.2%, | (10.2%, | (47.1%, | (28.4%, | (4.3%, | (40.0%, | (51.6%, | (32.9%, |
| | 57.9%) | 69.9%) | 48.4%) | 86.8%) | 99.5%) | 77.7%) | 97.2%) | 97.9%) | 63.1%) |
| Follicular lymphoma | N = 1 | N = 11 | N = 7 | N = 5 | N = 1 | N = 3 | N = 1 | N = 1 | N = 12 |
| Best overall response | | | | | | | | | |
| Complete response (CR) | 0 | 5 | 2 | 3 | 1 | 2 | 0 | 0 | 5 |
| | | (45.5%) | (28.6%) | (60.0%) | (100%) | (66.7%) | | | (41.7%) |
| Partial response (PR) | 0 | 4 | 3 | 1 | 0 | 1 | 0 | 0 | 4 |
| | | (36.4%) | (42.9%) | (20.0%) | | (33.3%) | | | (33.3%) |
| Stable disease (SD) | 1 | 0 | 1 | 0 | 0 | 0 | 0 | 0 | 1 |
| | (100%) | | (14.3%) | | | | | | (8.3%) |
| Progressive disease | 0 | 2 | 1 | 1 | 0 | 0 | 1 | 1 | 2 |
| | | (18.2%) | (14.3%) | (20.0%) | | | (100%) | (100%) | (16.7%) |
| Not evaluable | 0 | 0 | 0 | 0 | 0 | 0 | 0 | 0 | 0 |
| No assessments^{a} | 0 | 0 | 0 | 0 | 0 | 0 | 0 | 0 | 0 |
| Overall response rate (CR + PR) | 0 | 9 | 5 | 4 | 1 | 3 | 0 | 0 | 9 |
| (95% CI)^{b} | (0.0%, | (81.8%) | (71.4%) | (80.0%) | (100%) | (100%) | (0.0%, | (0.0%, | (75.0%) |
| | 97.5%) | (48.2%, | (29.0%, | (28.4%, | (2.5%, | (29.2%, | 97.5%) | 97.5%) | (42.8%, |
| | | 97.7%) | 96.3%) | 99.5%) | 100%) | 100%) | | | 94.5%) |
| Complete response rate | 0 | 5 | 2 | 3 | 1 | 2 | 0 | 0 | 5 |
| (95% CI)^{b} | (0.0%, | (45.5%) | (28.6%) | (60.0%) | (100%) | (66.7%) | (0.0%, | (0.0%, | (41.7%) |
| | 97.5%) | (16.7%, | (3.7%) | (14.7%, | (2.5%, | (9.4%, | 97.5%) | 97.5%) | (15.2%, |
| | | 76.6%) | 71.0%) | 94.7%) | 100%) | 99.2%) | | | 72.3%) |
| Disease control rate (CR + PR + | 1 | 9 | 6 | 4 | 1 | 3 | 0 | 0 | 10 |
| SD) | (100%) | (81.8%) | (85.7%) | (80.0%) | (100%) | (100%) | (0.0%, | (0.0%, | (83.3%) |
| (95% CI)^{b} | (2.5%, | (48.2%, | (42.1%, | (28.4%, | (2.5%, | (29.2%, | 97.5%) | 97.5%) | (51.6%, |
| | 100%) | 97.7%) | 99.6%) | 99.5%) | 100%) | 100%) | | | 97.9%) |

Full Analysis set for DLBCL and FL subjects. Of the 4 subjects enrolled with mantle-cell lymphoma, 2 responded (1 CR, 1 PR). The remaining subjects had other B-cell non-Hodgkin lymphoma histologies.
^{a}Subjects died without any post-baseline assessment.
^{b}Based on the Clopper and Pearson method.

In the expansion part, 38.9% of the heavily treated and high-risk patient population received prior CAR-T therapy, and 38.9% had received 4 or more lines of prior therapies. 61.1% of the population were primary refractory and 82.5% were refractory to the last therapy.

As of Jan. 31, 2022, the objective response rate (ORR (CR+PR) based on IRC assessment was 63.1% (95% CI: 55%, 70.6%), with 38.9% complete response (CR) and 24.2% partial response (PR)

In the following subgroups, based on IRC, ORR and CR rates were as follows:
OR rate of 54% (95% CI: 42%, 67%) and 34.4 CR rate in subjects with prior CAR-T experience (N=61).
OR rate 69% (95% CI: 59%, 78%) and 41.7% CR rate in subjects without prior CAR-T experience (N=96).

By IRC, median duration of response (mDOR) was 12 months. The estimated percentage of subjects remaining in response at 6 months was 62.2% (all responders) and was 60.8% at 9 months (all responders). For subjects achieving CR, the estimated percentage of subjects remaining in response at 6 months and 9 months was 88.7% and 79.1%, respectively. Median progression-free survival was 4.4 months (95CI; 3, 7.9).

Median overall survival was not reached. The estimated percentage of subjects alive at 9 months was 63.9% (95% CI: 55.6%, 71.1%).

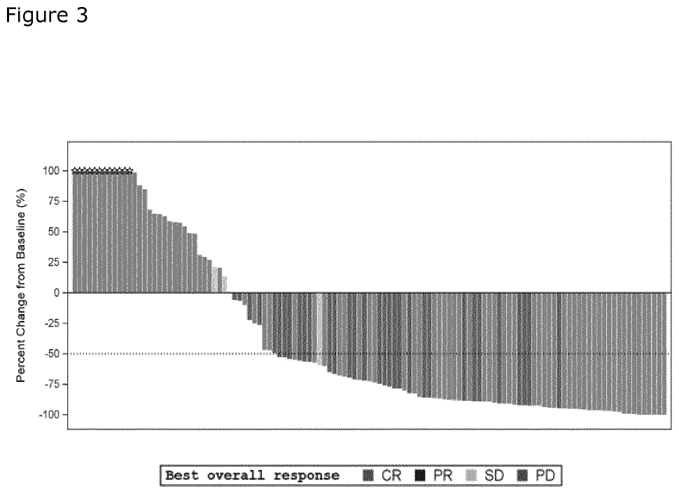

FIG. 3 shows a waterfall plot of best reduction in sum of the products of diameters based on IRC assessment according to Lugano criteria in the expansion aNHL cohort.

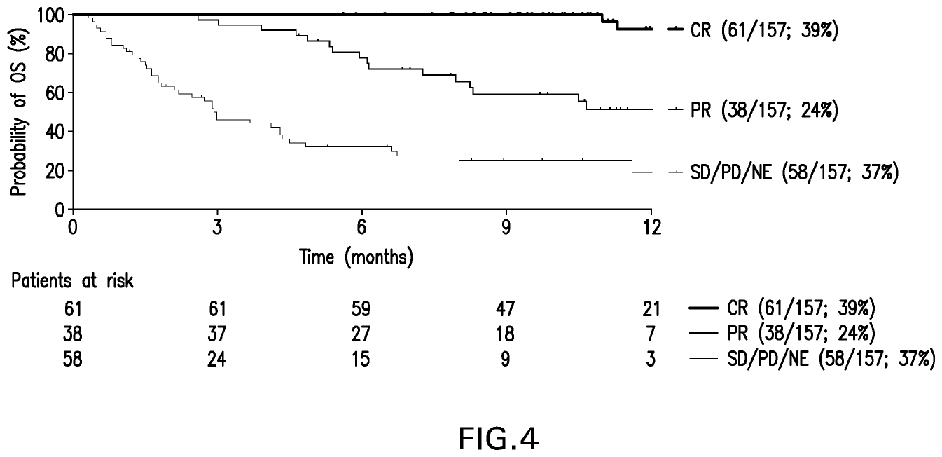

FIG. 4 shows Overall Survival by Best Overall Response. Median overall survival was not reached; CR patients had robust survival.

Data from further subgroup analyses are shown in Table 7.

TABLE 7
Responses across subgroups in GCT3013-01

| | | | | | | | Non- |
|---|---|---|---|---|---|---|---|
| | Overall | Primary | CAR-T | CAR-T | | Non- | DLBCL |
| | LBCL | refractory | naïve | exposed | DH/TH | DH/TH | subtypes^{a} |
| | (n = 157 | (n = 96) | (n = 96) | (n = 61) | (n = 12) | (n = 76) | (n = 30) |
| ORR % | 63 | 55 | 69 | 54 | 50 | 63 | 63 |
| CR Rate | 39 | 30 | 42 | 34 | 33 | 41 | 37 |
| % | | | | | | | |
| mDOR, | 12.0 | NR | 12.0 | 9.7 | 12.0 | NR | 12.0 |
| mo | | | | | | | |
| mDOR in | NR | NR | NR | NR | 12.0 | NR | 12.0 |
| CR pts, | | | | | | | |
| mo | | | | | | | |
| MRD | 45.4 | 40.3 | 44.6 | 46.7 | 36.4 | 50.8 | 36.0 |
| negativity | (54/119 | (29.72 | (33/74) | (21/45) | (4/11) | (32/63 | (9/25) |
| rate,^{b} % | | | | | | | |

^{a}includes HGBCL, PMBCL, and FL grade 3B.
^{b}MRD negativity rate is of June 2022
DH/TH = Double/Triple hit lymphoma

Minimal residual disease (MRD) negativity was correlated with improved progression free survival (PFS) (FIG. 7). MRD-positive patients had similar PFS across subgroups.

As of a data cutoff on Jun. 30, 2022, an estimated 61.2% and 55.1% of responders remained in response at 9 and 12 months, respectively. An estimated 88.7% and 79.1% of complete responders remained in response at 9 and 12 months, respectively.

A cohort of patients with DLBCL from the dose-escalation portion of EPCORE NHL-1 had extended follow-up: median, 21.1 months; longest follow-up, 26.7 months. Six patients remained on Q4W treatment (≥12 mg) and in CR. The median duration of CR was not reached.

### Safety and Tolerability

All 68 subjects in the GCT3013-01 dose escalation part had experienced at least 1 treatment-emergent adverse event (TEAE). The 4 most common TEAEs were pyrexia (69.1%), CRS (58.8%), injection site reaction (47.1%), and fatigue (44.1%). In total, 80.9% of subjects had at least one grade 3 or higher TEAE. There were 13 subjects with TEAEs leading to death (11 subjects with malignant neoplasm progression, 1 with euthanasia [also in the context of progressive disease], and 1 with COVID-19 [coronavirus disease 2019] pneumonia). None was considered related to epcoritamab. Serious adverse events (SAEs) were reported for 46 subjects (67.6%) of subjects. The most common SAE considered related to trial drug was pyrexia (19 subjects, 27.9%), which was reported as a symptom of CRS (see Section 4.6.2.2). The occurrence of TEAEs leading to permanent treatment discontinuation was low (13.2% of subjects). Adverse events of special interest (AESIs) included the following: 40 subjects (58.8%) experienced CRS, 4 subjects (5.9%) experienced neurological symptoms considered by the investigator to be immune mediated, and 1 subject (1.5%) experienced clinical tumor lysis syndrome. AESIs were captured on a separate AESI page in the eCRF. Symptoms relating to the AESI that met the seriousness criteria were reported as SAEs with the overall diagnosis as the main event. 46 of the 68 subjects (67.6%) treated in the GCT3013-01 dose escalation part had experienced at least 1 SAE. A total of 24 subjects (35.3%) experienced SAEs that were considered related to epcoritamab. The most common SAE considered related to trial drug was pyrexia (19 subjects, 27.9%), which was reported as a symptom of CRS.

286 of the 291 subjects (98.3%) in the GCT3013-01 expansion part had experienced at least 1 TEAE (Table 8). The 3 most common TEAEs were CRS (56.0%), fatigue (25.1%), and pyrexia (24.1%) (Table 9). In total, 59.8% of subjects had at least one grade 3 or higher TEAE (Table 10). There were 15 subjects with TEAEs leading to death. One subject experienced a TEAE leading to death that was considered related to epcoritamab by the investigator (immune effector cell-associated neurotoxicity syndrome [ICANS]). SAEs were reported for 60.5% of subjects. The most common SAE considered related to trial drug was CRS (56.0%). The occurrence of TEAEs leading to permanent treatment discontinuation was low (5.8% of subjects). AESIs included the following: 163 subjects (56.0%) experienced CRS, 21 subjects (7.2%) experienced ICANS, and 4 subject (1.4%) experienced clinical tumor lysis syndrome.

As said, 15 of the 291 subjects (5.2%) treated in the GCT3013-01 expansion part had experienced TEAEs leading to death. The one subject experienced a TEAE leading to death that was considered related to epcoritamab by the investigator. This subject was a 72-year-old female with stage IV non-GCB DLBCL and medical history of diabetes, hypertension, hyperlipidemia, as well as right upper lobectomy due to lung cancer. At screening, the subject had pancreatic, splenic, and para-aortic lymphoma involvement. The subject experienced continuous abdominal pain 2 days after priming dose of 0.16 mg of epcoritamab and was later diagnosed with grade 3 pancreatitis, treated with multiple repeated doses of morphine, and progressive lymphoma was determined based on imaging.

Four days after intermediate dose 0.8 mg of epcoritamab, grade 2 ICANS was reported, which later worsened to grade 4. In addition, grade 1 cerebral ischemia was observed at brain imaging. The study drug was withdrawn. The subject's condition deteriorated, and the subject expired 17 days after intermediate dose 0.8 mg of epcoritamab, with death reported to be due to ICANS. However, metabolic encephalopathy caused by hyperammonemia in a subject with long history of diabetes, hypertension, hyperlipidemia, possible microangiopathy (suggested by new multifocal cerebral infarcts, splenic infarct, laboratory evidence of coagulopathy, and renal dysfunction), as well as an accumulation of active metabolites of morphine use (which are dependent on functioning kidneys for clearance), are very likely confounding factors for the neurotoxicity. Unwarranted administration of tocilizumab to treat neurologic symptoms in the absence of CRS is known to increase the potential for neurotoxicity by increasing the circulating levels of IL-6, and likely contributed to the worsening of neurotoxicity in this case.

TABLE 8
Summary of Treatment-Emergent Adverse Events - GCT3013-01 Expansion Part

| Cohort | | | |
|---|---|---|---|
| aNHL | iNHL | MCL | Total |
| (N = 157) | (N = 101) | (N = 33) | (N = 291) |

Number (%) of subjects
with
at least 1

| TEAE | 155 | (98.7%) | 99 | (98.0%) | 32 | (97.0%) | 286 | (98.3%) |
|---|---|---|---|---|---|---|---|---|
| Related TEAE | 129 | (82.2%) | 94 | (93.1%) | 26 | (78.8%) | 249 | (85.3%) |
| Grade 3 and higher | 91 | (58.0%) | 60 | (59.4%) | 23 | (69.7%) | 174 | (59.8%) |
| TEAE | | | | | | | | |
| Grade 3 and higher | 43 | (27.4%) | 32 | (31.7%) | 13 | (39.4%) | 88 | (30.2%) |
| related TEAE | | | | | | | | |
| TEAE by worst toxicity | | | | | | | | |
| grade | | | | | | | | |
| Grade 1 | 21 | (13.4%) | 9 | (8.9%) | 2 | (6.1%) | 32 | (11.0%) |
| Grade 2 | 43 | (27.4%) | 30 | (29.7%) | 7 | (21.2%) | 80 | (27.5%) |
| Grade 3 | 54 | (34.4%) | 41 | (40.6%) | (15 | (45.5%) | 110 | (37.8%) |
| Grade 4 | 28 | (17.8%) | 15 | (14.9%) | 7 | (21.2%) | 50 | (17.2%) |
| Grade 5 | 9 | (5.7%) | 4 | (4.0%) | 1 | (3.0%) | 14 | (4.8%) |
| Serious TEAE | 88 | (56.1%) | 66 | (65.3%) | 22 | (66.7%) | 176 | (60.5%) |
| Serious related TEAE | 55 | (35.0%) | 54 | (53.5%) | 13 | (39.4%) | 122 | (41.9%) |
| TEAE leading to | 13 | (8.3%) | 2 | (2.0%) | 2 | (6.1%) | 17 | (5.89%) |
| treatment | | | | | | | | |
| discontinuation | | | | | | | | |
| TEAE leading to dose | 53 | (33.8%) | 52 | (51.5%) | 13 | (39.4%) | 118 | (40.5%) |
| modification | | | | | | | | |
| Fatal TEAE | 9 | (5.7%) | 5 | (5.0%) | 1 3.0%) | | 15 | (15.2%) |
| Adverse event of | | | | | | | | |
| special interest | | | | | | | | |
| Cytokine release | 77 | (49.0%) | 68 | (67.3%) | 18 | (54.5%) | 163 | (56.0%) |
| syndrome | | | | | | | | |
| ICANS | 9 | (5.7%) | 8 | (7.9%) | 4 | (12.1%) | 21 | (7.2%) |

| | | | | | | | |
|---|---|---|---|---|---|---|---|
| Clinical tumor lysis | 2 | (1.3%) | 0 | 2 | (6.1%) | 4 | (1.4%) |

syndrome

Note:
Percentages calculated based on number of subjects in the safety set.
Adverse events are classified using the Medical Dictionary for Regulatory Activities (MedDRA) v23.1 and Common Terminology Criteria for Adverse Events (CTCAE) v5.0, and are counted only once per category. Cytokine release syndrome is graded per (Lee et al., 2019), and clinical tumor lysis syndrome according to Cairo-Bishop (Coiffier et al., 2008).
aNHL = aggressive B-cell non-Hodgkin lymphoma; ICANS = immune effector cell-associated neurotoxicity syndrome; iNHL = indolent B-cell non-Hodgkin lymphoma; TEAE = treatment-emergent adverse event.

TABLE 9
Treatment-Emergent Adverse Events Occurring in >10% of Subjects - GCT3013-01 Expansion Part

| | Cohort | | | |
|---|---|---|---|---|
| | aNHL | iNHL | MCL | Total |
| Preferred Term | (N = 157) | (N = 101) | (N = 33) | (N = 291) |
| Number of subjects with at least one TEAE | 155 (98.7%) | 99 (98.0%) | 32 (97.0%) | 286 (98.3%) |
| Cytokine release syndrome | 77 (49.0%) | 68 (67.3%) | 18 (54.5%) | 163 (56.0%) |
| Fatigue | 36 (22.9%) | 30 (29.7%) | 7 (21.2%) | 73 (25.1%) |
| Pyrexia | 37 (23.6%) | 27 (26.7%) | 6 (18.2%) | 70 (24.1%) |
| Injection site reaction | 30 (19.1%) | 38 (37.6%) | 1 (3.0%) | 69 (23.7%) |
| Neutropenia | 31 (19.7%) | 20 (19.8%) | 7 (21.2%) | 58 (19.9%) |
| Diarrhoea | 32 (20.4%) | 19 (18.8%) | 2 (6.1%) | 53 (18.2%) |
| Nausea | 31 (19.7%) | 14 (13.9%) | 5 (15.2%) | 50 (17.2%) |
| Anaemia | 27 (17.2%) | 12 (11.9%) | 6 (18.2%) | 45 (15.5%) |
| Headache | 21 (13.4%) | 15 (14.9%) | 2 (6.1%) | 38 (13.1%) |
| Constipation | 19 (12.1%) | 11 (10.9%) | 5 (15.2%) | 35 (12.0%) |
| Thrombocytopenia | 20 (12.7%) | 8 (7.9%) | 3 (9.1%) | 31 (10.7%) |

Note:
Percentages calculated based on number of subjects in Safety Set.
Note:
Adverse events are classified using MedDRA 24.1, and are counted at most one time per preferred term.
Snapshot date: 31 JAN. 2022; Data Cutoff: 31 JAN. 2022.

TABLE 10
Treatment-Emergent Adverse Events of Grade 3 or Higher, by System
Organ Class and Preferred Term - GCT3013-01 Expansion Part

| | Cohort | | | |
|---|---|---|---|---|
| System Organ Class/ | aNHL | iNHL | MCL | Total |
| Preferred Term | (N = 157) | (N = 101) | (N = 33) | (N = 291) |
| Number of subjects with at least one grade 3 or higher TEAE | 91 (58.0%) | 60 (59.4%) | 23 (69.7%) | 174 (59.8%) |
| Blood and lymphatic system disorders | 42 (26.8%) | 25 (24.8%) | 12 (36.4%) | 79 (27.1%) |
| Neutropenia | 20 (12.7%) | 17 (16.8%) | 7 (21.2%) | 44 (15.1%) |
| Anaemia | 15 (9.6%) | 5 (5.0%) | 4 (12.1%) | 24 (8.2%) |
| Thrombocytopenia | 9 (5.7%) | 5 (5.0%) | 3 (9.1%) | 17 (5.8%) |
| Lymphopenia | 6 (3.8%) | 1 (1.0%) | 0 | 7 (2.4%) |
| Febrile neutropenia | 4 (2.5%) | 0 | 0 | 4 (1.4%) |
| Leukopenia | 1 (0.6%) | 0 | 2 (6.1%) | 3 (1.0%) |
| Lymph node pain | 0 | 1 (1.0%) | 0 | 1 (0.3%) |
| Lymphocytosis | 1 (0.6%) | 0 | 0 | 1 (0.3%) |
| Infections and infestations | 24 (15.3%) | 22 (21.8%) | 7 (21.2%) | 53 (18.2%) |
| COVID-19 | 5 (3.2%) | 6 (5.9%) | 0 | 11 (3.8%) |
| Pneumonia | 1 (0.6%) | 4 (4.0%) | 1 (3.0%) | 6 (2.1%) |
| Sepsis | 4 (2.5%) | 0 | 1 (3.0%) | 5 (1.7%) |
| Cellulitis | 3 (1.9%) | 0 | 1 (3.0%) | 4 (1.4%) |
| Pneumocystis jirovecii pneumonia | 0 | 3 (3.0%) | 0 | 3 (1.0%) |
| Septic shock | 2 (1.3%) | 1 (1.0%) | 0 | 3 (1.0%) |
| Urinary tract infection | 0 | 3 (3.0%) | 0 | 3 (1.0%) |
| Cytomegalovirus infection | 1 (0.6%) | 1 (1.0%) | 0 | 2 (0.7%) |
| Herpes zoster | 0 | 1 (1.0%) | 1 (3.0%) | 2 (0.7%) |
| Progressive multifocal leukoencephalopathy | 2 (1.3%) | 0 | 0 | 2 (0.7%) |
| Pyelonephritis | 1 (0.6%) | 1 (1.0%) | 0 | 2 (0.7%) |
| Upper respiratory tract infection | 2 (1.3%) | 0 | 0 | 2 (0.7%) |
| Infections and infestations (Cont.) | 24 (15.3%) | 22 (21.8%) | 7 (21.2%) | 53 (18.2%) |
| Bacteraemia | 1 (0.6%) | 0 | 0 | 1 (0.3%) |
| Bronchitis | 0 | 0 | 1 (3.0%) | 1 (0.3%) |
| Cytomegalovirus infection reactivation | 0 | 1 (1.0%) | 0 | 1 (0.3%) |
| Device related infection | 1 (0.6%) | 0 | 0 | 1 (0.3%) |
| Enterococcal infection | 1 (0.6%) | 0 | 0 | 1 (0.3%) |
| Escherichia infection | 0 | 0 | 1 (3.0%) | 1 (0.3%) |
| Escherichia urinary tract infection | 1 (0.6%) | 0 | 0 | 1 (0.3%) |
| Gastroenteritis viral | 0 | 1 (1.0%) | 0 | 1 (0.3%) |
| Infectious pleural effusion | 0 | 1 (1.0%) | 0 | 1 (0.3%) |
| Infective exacerbation of bronchiectasis | 0 | 1 (1.0%) | 0 | 1 (0.3%) |
| Klebsiella bacteraemia | 0 | 0 | 1 (3.0%) | 1 (0.3%) |
| Localised infection | 0 | 1 (1.0%) | 0 | 1 (0.3%) |
| Meningitis | 0 | 1 (1.0%) | 0 | 1 (0.3%) |
| Necrotising fasciitis | 0 | 1 (1.0%) | 0 | 1 (0.3%) |
| Pharyngitis | 0 | 1 (1.0%) | 0 | 1 (0.3%) |
| Pneumonia aspiration | 0 | 0 | 1 (3.0% | 1 (0.3%) |
| Respiratory tract infection | 1 (0.6%) | 0 | 0 | 1 (0.3%) |
| Rhinovirus infection | 0 | 0 | 1 (1.0%) | 1 (0.3%) |
| Skin infection | 1 (0.6%) | 0 | 0 | 1 (0.3%) |
| Staphylococcal bacteraemia | 1 (0.6%) | 0 | 0 | 1 (0.3%) |
| Staphylococcal infection | 0 | 1 (1.0%) | 0 | 1 (0.3%) |
| Urinary tract infection enterococcal | 0 | 1 (1.0%) | 0 | 1 (0.3%) |
| Urinary tract infection pseudomonal | 1 (0.6%) | 0 | 0 | 1 (0.3%) |
| Infections and infestations (Cont.) | 24 (15.3%) | 22 (21.8%) | 7 (21.2%) | 53 (18.2%) |
| Urosepsis | 1 (0.6%) | 0 | 0 | 1 (0.3%) |
| Varicella zoster virus infection | 0 | 1 (1.0%) | 0 | 1 (0.3%) |
| Investigations | 19 (12.1%) | 15 (14.9%) | 4 (12.1%) | 38 (13.1%) |
| Neutrophil count decreased | 10 (6.4%) | 5 (5.0%) | 1 (3.0%) | 16 (5.5%) |
| Platelet count decreased | 2 (1.3%) | 1 (1.0%) | 3 (9.1%) | 6 (2.1%) |
| Alamine aminotransferase increased | 1 (0.6%) | 2 (2.0%) | 0 | 3 (1.0%) |
| Aspartate aminotransferase increased | 2 (1.3%) | 1 (1.0%) | 0 | 3 (1.0%) |
| C-reactive protein increased | 1 (0.6%) | 2 (2.0%) | 0 | 3 (1.0%) |
| Lymphocyte count decreased | 1 (0.6%) | 1 (1.0%) | 0 | 2 (0.7%) |
| Blood bilirubin increased | 0 | 1 (1.0%) | 0 | 1 (0.3%) |
| Blood glucose increased | 1 (0.6%) | 0 | 0 | 1 (0.3%) |
| Blood lactate dehydrogenase increased | 1 (0.6%) | 0 | 0 | 1 (0.3%) |
| Blood sodium decreased | 1 (0.6%) | 0 | 0 | 1 (0.3%) |
| CD4 lymphocytes decreased | 0 | 1 (1.0%) | 0 | 1 (0.3%) |
| Electrocardiogram OT prolonged | 0 | 1 (1.0%) | 0 | 1 (0.3%) |
| Gamma-glutamyltransferase decreased | 0 | 1 (1.0%) | 0 | 1 (0.3%) |
| Gamma-glutamyltransferase increased | 1 (0.6%) | 0 | 0 | 1 (0.3%) |
| Transaminases increased | 0 | 1 (1.0%) | 0 | 1 (0.3%) |
| Metabolism and nutrition disorders | 11 (7.0%) | 12 (11.9%) | 5 (15.2%) | 28 (9.6%) |
| Hyperglycaemia | 3 (1.9%) | 4 (4.0%) | 1 (3.0%) | 8 (2.7%) |
| Type 2 diabetes mellitus | 0 | 4 (4.0%) | 0 | 4 (1.4%) |
| Tumour lysis syndrome | 2 (1.3%) | 0 | 1 (3.0%) | 3 (1.0%) |
| Decreased appetite | 1 (0.6%) | 0 | 1 (3.0%) | 2 (0.7%) |
| Metabolism and nutrition disorders (Cont.) | 11 (7.0%) | 12 (11.9%) | 5 (15.2%) | 28 (9.6%) |
| Hypercalcaemia | 1 (0.6%) | 1 (1.0%) | 0 | 2 (0.7%) |
| Hypokalaemia | 1 (0.6%) | 0 | 1 (3.0%) | 2 (0.7%) |
| Hypophosphataemia | 2 (1.3%) | 0 | 0 | 2 (0.7%) |
| Steroid diabetes | 2 (1.3%) | 0 | 0 | 2 (0.7%) |
| Dehydration | 0 | 1 (1.0%) | 0 | 1 (0.3%) |
| Hyperuricaemia | 0 | 0 | 1 (3.0%) | 1 (0.3%) |
| Hypomagnesaemia | 0 | 1 (1.0%) | 0 | 1 (0.3%) |
| Hyponatraemia | 0 | 0 | 1 (3.0%) | 1 (0.3%) |
| Malnutrition | 0 | 1 (1.0%) | 0 | 1 (0.3%) |
| Metabolic acidosis | 1 (0.6%) | 0 | 0 | 1 (0.3%) |
| Respiratory, thoracic and mediastinal disorders | 11 (7.0%) | 6 (5.9%) | 2 (6.1%) | 19 (6.5%) |
| Pleural effusion | 6 (3.8%) | 0 | 2 (6.1%) | 8 (2.7%) |
| Dyspnoea | 4 (2.5%) | 1 (1.0%) | 0 | 5 (1.7%) |
| Pulmonary embolism | 1 (0.6%) | 3 (3.0%) | 0 | 4 (1.4%) |
| Acute respiratory failure | 0 | 1 (1.0%) | 0 | 1 (0.3%) |
| Lung opacity | 0 | 1 (1.0%) | 0 | 1 (0.3%) |
| Respiratory failure | 1 (0.6%) | 0 | 0 | 1 (0.3%) |
| General disorders and administration site conditions | 7 (4.5%) | 6 (5.9%) | 4 (12.1%) | 17 (5.8%) |
| Fatigue | 3 (1.9%) | 2 (2.0%) | 1 (3.0%) | 6 (2.1%) |
| Generalised oedema | 1 (0.6%) | 0 | 1 (3.0%) | 2 (0.7%) |
| Malaise | 1 (0.6%) | 1 (1.0%) | 0 | 2 (0.7%) |
| Oedema | 2 (1.3%) | 0 | 0 | 2 (0.7%) |
| Pyrexia | 0 | 2 (2.0%) | 0 | 2 (0.7%) |
| General disorders and administration site conditions (Cont.) | 7 (4.5%) | 6 (5.9%) | 4 (12.1%) | 17 (5.8%) |
| Asthenia | 0 | 1 (1.0%) | 0 | 1 (0.3%) |
| Facial pain | 0 | 0 | 1 (3.0%) | 1 (0.3%) |
| General physical health deterioration | 1 (0.6%) | 0 | 0 | 1 (0.3%) |
| Multiple organ dysfunction syndrome | 0 | 0 | 1 (3.0%) | 1 (0.3%) |
| Pain | 0 | 0 | 1 (3.0%) | 1 (0.3%) |
| Gastrointestinal disorders | 8 (5.1%) | 2 (2.0%) | 4 (12.1%) | 14 (4.8%) |
| Abdominal pain | 4 (2.5%) | 1 (1.0%) | 1 (3.0%) | 6 (2.1%) |
| Nausea | 2 (1.3%) | 0 | 0 | 2 (0.7%) |
| Abdominal mass | 0 | 0 | 1 (3.0%) | 1 (0.3%) |
| Diarrhoea | 0 | 0 | 1 (3.0%) | 1 (0.3%) |
| Enteritis | 0 | 1 (1.0%) | 0 | 1 (0.3%) |
| Pancreatitis | 1 (0.6%) | 0 | 0 | 1 (0.3%) |
| Upper gastrointestinal haemorrhage | 0 | 0 | 1 (3.0%) | 1 (0.3%) |
| Vomiting | 1 (0.6%) | 0 | 0 | 1 (0.3%) |
| Nervous system disorders | 8 (5.1%) | 2 (2.0%) | 1 (3.0%) | 11 (3.8%) |
| Headache | 1 (0.6%) | 0 | 1 (3.0%) | 2 (0.7%) |
| Acute polyneuropathy | 1 (0.6%) | 0 | 0 | 1 (0.3%) |
| Bell's palsy | 0 | 1 (1.0%) | 0 | 1 (0.3%) |
| Cerebral haemorrhage | 1 (0.6%) | 0 | 0 | 1 (0.3%) |
| Chronic lymphocytic inflammation with pontine perivascular | 1 (0.6%) | 0 | 0 | 1 (0.3%) |
| enhancement responsive to steroids | | | | |
| Facial paralysis | 1 (0.6%) | 0 | 0 | 1 (0.3%) |
| Hydrocephalus | 1 (0.6%) | 0 | 0 | 1 (0.3%) |
| Immune effector cell-associated neurotoxicity syndrome | 1 (0.6%) | 0 | 0 | 1 (0.3%) |
| Nervous system disorders (Cont.) | 8 (5.1%) | 2 (2.0%) | 1 (3.0%) | 11 (3.8%) |
| Spinal cord compression | 1 (0.6%) | 0 | 0 | 1 (0.3%) |
| Syncope | 0 | 1 (1.0%) | 0 | 1 (0.3%) |
| Immune system disorders | 3 (1.9%) | 3 (3.0%) | 4 (12.1%) | 10 (3.4%) |
| Cytokine release syndrome | 3 (1.9%) | 3 (3.0%) | 4 (12.1%) | 10 (3.4%) |
| Cardiac disorders | 6 (3.8%) | 1 (1.0%) | 2 (6.1%) | 9 (3.1%) |
| Atrial fibrillation | 0 | 1 (1.0%) | 1 (3.0%) | 2 (0.7%) |
| Cardiac failure | 1 (0.6%) | 0 | 0 | 1 (0.3%) |
| Cardiac failure chronic | 1 (0.6%) | 0 | 0 | 1 (0.3%) |
| Cardiac tamponade | 1 (0.6%) | 0 | 0 | 1 (0.3%) |
| Myocardial infarction | 1 (0.6%) | 0 | 0 | 1 (0.3%) |
| Pericardial effusion | 1 (0.6%) | 0 | 0 | 1 (0.3%) |
| Sinus node dysfunction | 0 | 0 | 1 (3.0%) | 1 (0.3%) |
| Sinus tachycardia | 1 (0.6%) | 0 | 0 | 1 (0.3%) |
| Injury, poisoning and procedural complications | 4 (2.5%) | 2 (2.0%) | 1 (3.0%) | 7 (2.4%) |
| Fall | 0 | 1 (1.0%) | 1 (3.0%) | 2 (0.7%) |
| Femoral neck fracture | 1 (0.6%) | 0 | 0 | 1 (0.3%) |
| Infusion related reaction | 0 | 1 (1.0%) | 0 | 1 (0.3%) |
| Recall phenomenon | 1 (0.6%) | 0 | 0 | 1 (0.3%) |
| Spinal shock | 1 (0.6%) | 0 | 0 | 1 (0.3%) |
| Subdural haematoma | 1 (0.6%) | 0 | 0 | 1 (0.3%) |
| Neoplasms benign, malignant and unspecified (incl cysts and polyps) | 5 (3.2%) | 2 (2.0%) | 0 | 7 (2.4%) |
| Infected neoplasm | 1 (0.6%) | 0 | 0 | 1 (0.3%) |
| Lung neoplasm | 1 (0.6%) | 0 | 0 | 1 (0.3%) |
| Neoplasms benign, malignant and unspecified (incl cysts and polyps) | 5 (3.2%) | 2 (2.0%) | 0 | 7 (2.4%) |
| (Cont.) | | | | |
| Lymphoma transformation | 0 | 1 (1.0%) | 0 | 1 (0.3%) |
| Myelodysplastic syndrome | 1 (0.6%) | 0 | 0 | 1 (0.3%) |
| Refractory cytopenia with multilineage dysplasia | 1 (0.6%) | 0 | 0 | 1 (0.3%) |
| Squamous cell carcinoma | 0 | 1 (1.0%) | 0 | 1 (0.3%) |
| Tumour pain | 1 (0.6%) | 0 | 0 | 1 (0.3%) |
| Musculoskeletal and connective tissue disorders | 6 (3.8%) | 0 | 0 | 6 (2.1%) |
| Arthralgia | 1 (0.6%) | 0 | 0 | 1 (0.3%) |
| Back pain | 1 (0.6%) | 0 | 0 | 1 (0.3%) |
| Fistula | 1 (0.6%) | 0 | 0 | 1 (0.3%) |
| Groin pain | 1 (0.6%) | 0 | 0 | 1 (0.3%) |
| Muscular weakness | 1 (0.6%) | 0 | 0 | 1 (0.3%) |
| Pain in extremity | 1 (0.6%) | 0 | 0 | 1 (0.3%) |
| Vascular disorders | 5 (3.2%) | 0 | 1 (3.0%) | 6 (2.1%) |
| Hypotension | 2 (1.3%) | 0 | 0 | 2 (0.7%) |
| Haematoma | 1 (0.6%) | 0 | 0 | 1 (0.3%) |
| Hypertension | 1 (0.6%) | 0 | 0 | 1 (0.3%) |
| Hypovolaemic shock | 0 | 0 | 1 (3.0%) | 1 (0.3%) |
| Thrombophlebitis | 1 (0.6%) | 0 | 0 | 1 (0.3%) |
| \|Renal and urinary disorders | 2 (1.3%) | 2 (2.0%) | 1 (3.0%) | 5 (1.7%) |
| Acute kidney injury | 2 (1.3%) | 2 (2.0%) | 1 (3.0%) | 5 (1.7%) |
| Hepatobiliary disorders | 2 (1.3%) | 1 (1.0%) | 0 | 3 (1.0%) |
| Cholestasis | 0 | 1 (1.0%) | 0 | 1 (0.3%) |
| Hepatotoxicity | 1 (0.6%) | 0 | 0 | 1 (0.3%) |
| Liver injury | 1 (0.6%) | 0 | 0 | 1 (0.3%) |
| Psychiatric disorders | 2 (1.3%) | 0 | 1 (3.0%) | 3 (1.0%) |
| Anhedonia | 0 | 0 | 1 (3.0%) | 1 (0.3%) |
| Delirium | 1 (0.6%) | 0 | 0 | 1 (0.3%) |
| Emotional distress | 0 | 0 | 1 (3.0%) | 1 (0.3%) |
| Insomnia | 1 (0.6%) | 0 | 0 | 1 (0.3%) |
| Skin and subcutaneous tissue disorders | 1 (0.6%) | 1 (1.0%) | 1 (3.0%) | 3 (1.0%) |
| Pruritus | 0 | 0 | 1 (3.0%) | 1 (0.3%) |
| Rash | 0 | 1 (1.0%) | 0 | 1 (0.3%) |
| Skin lesion | 1 (0.6%) | 0 | 0 | 1 (0.3%) |
| Ear and labyrinth disorders | 0 | 1 (1.0%) | 0 | 1 (0.3%) |
| Deafness | 0 | 1 (1.0%) | 0 | 1 (0.3%) |
| Eye disorders | 1 (0.6%) | 0 | 0 | 1 (0.3%) |
| Blindness | 1 (0.6%) | 0 | 0 | 1 (0.3%) |
| Uncoded | 2 (1.3%) | 1 (1.0%) | 0 | 3 (1.0%) |
| Uncoded (Febrile neutrophenia due to bacteremia (staphylococcus | 0 | 1 (1.0%) | 0 | 1 (0.3%) |
| epidermidis)) | | | | |
| Uncoded (Luekopenia worsening) | 1 (0.6%) | 0 | 0 | 1 (0.3%) |
| Uncoded (Reduced neutrophil count) | 1 (0.6%) | 0 | 0 | 1 (0.3%) |

Note:
Percentages calculated based on number of subjects in Safety Set.
Note:
Adverse events are classified using MedDRA 24.1 and are counted at most one time per system organ class and at most one time per preferred term.
Snapshot date: 31 JAN. 2022; Data Cutoff: 31 JAN. 2022.

174 of the 291 subjects (59.8%) treated in the GCT3013-01 expansion part had experienced at least 1 SAE (Table 10). A total of 122 subjects (41.9%) experienced SAEs considered related to epcoritamab. The most common SAE considered related to trial drug was CRS (108 subjects, 37.1%) (Table 11).

TABLE 11
Serious Treatment-Emergent Adverse Events Considered Related to Epcoritamab,
by System Organ Class and Preferred Term - GCT3013-01 Expansion Part

| | Cohort | | | |
|---|---|---|---|---|
| System Organ Class/ | aNHL | iNHL | MCL | Total |
| Preferred Term | (N = 157) | (N = 101) | (N = 33) | (N = 291) |
| Number of subjects with at least one drug related serious TEAE | 55 (35.0%) | 54 (53.5%) | 13 (39.4%) | 122 (41.9%) |
| Immune system disorders | 46 (29.3%) | 51 (50.5%) | 11 (33.3%) | 108 (37.1%) |
| Cytokine release syndrome | 46 (29.3%) | 51 (50.5%) | 11 (33.3%) | 108 (37.1%) |
| Nervous system disorders | 5 (3.2%) | 5 (5.0%) | 2 (6.1%) | 12 (4.1%) |
| Immune effector cell-associated neurotoxicity syndrome | 4 (2.5%) | 3 (3.0%) | 1 (3.0%) | 8 (2.7%) |
| Headache | 0 | 1 (1.0%) | 1 (3.0%) | 2 (0.7%) |
| Chronic lymphocytic inflammation with pontine perivascular | 1 (0.6%) | 0 | 0 | 1 (0.3%) |
| enhancement responsive to steroids | | | | |
| Dizziness | 0 | 1 (1.0%) | 0 | 1 (0.3%) |
| Infections and infestations | 2 (1.3%) | 6 (5.9%) | 1 (3.0%) | 9 (3.1%) |
| Pneumonia | 0 | 2 (2.0%) | 0 | 2 (0.7%) |
| Sepsis | 1 (0.6%) | 0 | 1 (3.0%) | 2 (0.7%) |
| Cytomegalovirus infection reactivation | 0 | 1 (1.0%) | 0 | 1 (0.3%) |
| Infectious pleural effusion | 0 | 1 (1.0%) | 0 | 1 (0.3%) |
| Meningitis | 0 | 1 (1.0%) | 0 | 1 (0.3%) |
| Oral herpes | 1 (0.6%) | 0 | 0 | 1 (0.3%) |
| Pneumocystis jirovecii pneumonia | 0 | 1 (1.0%) | 0 | 1 (0.3%) |
| Upper respiratory tract infection | 1 (0.6%) | 0 | 0 | 1 (0.3%) |
| General disorders and administration site conditions | 0 | 5 (5.0%) | 1 (3.0%) | 6 (2.1%) |
| Fatigue | 0 | 2 (2.0%) | 0 | 2 (0.7%) |
| Pyrexia | 0 | 2 (2.0%) | 0 | 2 (0.7%) |
| Malaise | 0 | 1 (1.0%) | 0 | 1 (0.3%) |
| Multiple organ dysfunction syndrome | 0 | 0 | 1 (3.0%) | 1 (0.3%) |
| Blood and lymphatic system disorders | 3 (1.9%) | 0 | 1 (3.0%) | 1 (1.4%) |
| Thrombocytopenia | 1 (0.6%) | 0 | 1 (3.0%) | 2 (0.7%) |
| Febrile neutropenia | 1 (0.6%) | 0 | 0 | 1 (0.3%) |
| Lymphadenopathy | 1 (0.6%) | 0 | 0 | 1 (0.3%) |
| Gastrointestinal disorders | 2 (1.3%) | 1 (1.0%) | 0 | 1 (0.3%) |
| Abdominal pain | 0 | 1 (1.0%) | 0 | 1 (0.3%) |
| Nausea | 1 (0.6%) | 0 | 0 | 1 (0.3%) |
| Pancreatitis | 1 (0.6%) | 0 | 0 | 1 (0.3%) |
| Neoplasms benign, malignant and unspecified (incl cysts and polyps) | 3 (1.9%) | 0 | 0 | 3 (1.0%) |
| Tumour pain | 2 (1.3%) | 0 | 0 | 2 (0.7%) |
| Tumour flare | 1 (0.6%) | 0 | 0 | 1 (0.3%) |
| Respiratory, thoracic and mediastinal disorders | 2 (1.3%) | 1 (1.0%) | 0 | 3 (1.0%) |
| Pleural effusion | 1 (0.6%) | 1 (1.0%) | 0 | 2 (0.7%) |
| Dyspnoea | 1 (0.6%) | 0 | 0 | 1 (0.3%) |
| Investigations | 1 (0.6%) | 1 (1.0%) | 0 | 2 (0.7%) |
| C-reactive protein increased | 1 (0.6%) | 1 (1.0%) | 0 | 2 (0.7%) |
| Metabolism and nutrition disorders | 1 (0.6%) | 1 (1.0%) | 0 | 2 (0.7%) |
| Decreased appetite | 0 | 1 (1.0%) | 0 | 1 (0.3%) |
| Hypoalbuminaemia | 1 (0.6%) | 0 | 0 | 1 (0.3%) |
| Renal and urinary disorders | 0 | 1 (1.0%) | 1 (3.0%) | 2 (0.7%) |
| Acute kidney injury | 0 | 1 (1.0%) | 1 (3.0%) | 2 (0.7%) |
| Vascular disorders | 1 (0.6%) | 0 | 0 | 1 (0.3%) |
| Hypotension | 1 (0.6%) | 0 | 0 | 1 (0.3%) |

Note:
Percentages calculated based on number of subjects in Safety Set.
Note:
Adverse events are classified using MedDRA 24.1 and are counted at most one time per system organ class and at most one time per preferred term.
Snapshot date: 31 JAN. 2022; Data Cutoff: 31 JAN. 2022.

A total of 17 of the 291 subjects (5.8%) treated in the GCT3013-01 expansion part had experienced TEAEs leading to permanent discontinuation of trial drug: 2 subjects each experienced COVID-19 and cytokine release syndrome, 1 subject each experienced Pneumocystis jirovecii pneumonia, progressive multifocal leukoencephalopathy, sepsis, infected neoplasm, lung neoplasm, myelodysplastic syndrome, refractory cytopenia with multilineage dysplasia, fatigue, general physical health deterioration, multiple organ dysfunction syndrome, chronic lymphocytic inflammation with pontine perivascular enhancement responsive to steroids, immune effector cell-associated neurotoxicity syndrome, pleural effusion, respiratory failure, deafness, and hypercalcemia (Table 12).

TABLE 12
Treatment-Emergent Adverse Events Leading to Permanent Discontinuation of Treatment,
by System Organ Class and Preferred Term - GCT3013-01 Expansion Part.

| | Cohort | | | |
|---|---|---|---|---|
| System Organ Class/ | aNHL | iNHL | MCL | Total |
| Preferred Term | (N = 157) | (N = 101) | (N = 33) | (N = 291) |
| Number of subjects with at least one TEAE leading to permanent | 13 (8.3%) | 2 (2.0%) | 2 (6.1%) | 17 (5.8%) |
| treatment discontinuation | | | | |
| Infections and infestations | 3 (1.9%) | 1 (1.0%) | 1 (3.0%) | 5 (1.7%) |
| COVID-19 | 2 (1.3%) | 0 | 0 | 2 (0.7%) |
| Pneumocystis jirovecii pneumonia | 0 | 1 (1.0%) | 0 | 1 (0.3%) |
| Progressive multifocal leukoencephalopathy | 1 (0.6%) | 0 | 0 | 1 (0.3%) |
| Sepsis | 0 | 0 | 1 (3.0%) | 1 (0.3%) |
| Neoplasms benign, malignant and unspecified (incl cysts and polyps) | 4 (2.5%) | 0 | 0 | 4 (1.4%) |
| Infected neoplasm | 1 (0.6%) | 0 | 0 | 1 (0.3%) |
| Lung neoplasm | 1 (0.6%) | 0 | 0 | 1 (0.3%) |
| Myelodysplastic syndrome | 1 (0.6%) | 0 | 0 | 1 (0.3%) |
| Refractory cytopenia with multilineage dysplasia | 1 (0.6%) | 0 | 0 | 1 (0.3%) |
| General disorders and administration site conditions | 2 (1.3%) | 0 | 1 (3.0%) | 3 (1.0%) |
| Fatigue | 1 (0.6%) | 0 | 0 | 1 (0.3%) |
| General physical health deterioration | 1 (0.6%) | 0 | 0 | 1 (0.3%) |
| Multiple organ dysfunction syndrome | 0 | 0 | 1 (3.0%) | 1 (0.3%) |
| Immune system disorders | 1 (0.6%) | 0 | 1 (3.0%) | 2 (0.7%) |
| Cytokine release syndrome | 1 (0.6%) | 0 | 1 (3.0%) | 2 (0.7%) |
| Nervous system disorders | 2 (1.3%) | 0 | 0 | 2 (0.7%) |
| Chronic lymphocytic inflammation with pontine perivascular | 1 (0.6%) | 0 | 0 | 1 (0.3%) |
| enhancement responsive to steroids | | | | |
| Immune effector cell-associated neurotoxicity syndrome | 1 (0.6%) | 0 | 0 | 1 (0.3%) |
| Respiratory, thoracic and mediastinal disorders | 2 (1.3%) | 0 | 0 | 2 (0.7%) |
| Pleural effusion | 1 (0.6%) | 0 | 0 | 1 (0.3%) |
| Respiratory failure | 1 (0.6%) | 0 | 0 | 1 (0.3%) |

### Adverse Events of Special Interest (AESIs)

The following is a summary of epcoritamab AESIs:

Cytokine release syndrome, was reported in 209 subjects, including 40 subjects in the GCT3013-01 dose escalation part, 163 subjects in the GCT3013-01 expansion part, and 6 subjects in the GCT3013-04 dose escalation part. CRS was graded according to (Lee et al., 2019). All CRS events were grade 1 or 2, except for 2 events in the GCT3013-01 expansion part and 1 event in the GCT3013-04 dose escalation part that were grade 3.

Neurotoxicity is listed in the epcoritamab protocols as an AESI. In the dose escalation part of GCT3013-01 trial, neurological assessment was conducted according to CARTOX-10 scale (Neelapu et al., 2017). Suspected immune-mediated neurologic symptoms consistent with ICANS were captured as separate AESIs. In the expansion part of the GCT3013-01 trial and the GCT3013-04 trial, the ICANS cases are reported using the exact term of ICANS. Neurological symptoms reported as AESIs were reported in 29 subjects in GCT3013-01, including 4 subjects in the dose escalation part, and 25 subjects in the expansion part.

Clinical tumor lysis syndrome was reported in 5 subjects, including 1 subject in the GCT3013-01 dose escalation part, 4 subjects in the GCT3013-01 expansion part, and no subjects in the GCT3013-04 dose escalation part. Clinical tumor lysis syndrome was graded according to Cairo-Bishop (Coiffier et al., 2008).

### GCT3013-04 (NCT04542824)

GCT3013-04 is a phase I/II Open-Label trial cohort investigating the safety and preliminary efficacy of epcoritamab in Japanese Subjects with relapsed or refractory (R/R) B-NHL. Patients have been treated in the dose escalation and expansion parts, wherein patients receive treatment with epcoritamab subcutaneously in 28-day cycles, with in cycle 1; day 1, 0.16 mg, day 8 0.8 mg, days 15 and 22, 48 mg; cycles 2-3, day 1, 8, 15 and 22, 48 mg; cycles 4-9, day 1 and 15, 48 mg; and cycles 10+, 48 mg on day 1.

### Inclusion Criteria—Dose Escalation and Expansion

Each potential subject must fulfill all of the following criteria to be enrolled in the trial.
1. Subject (or their legally acceptable representative) must sign an ICF indicating that he or she understands the purpose of and procedures required for the trial and are willing to participate in the trial prior to any other trial related assessments or procedures.
2. Must be at least 20 years of age, inclusive.
3. Asian race and Japanese ethnicity.
4. Documented mature B-cell neoplasm according to the 2008 WHO classification or the 2016 WHO revision, depending on time of diagnosis.
   a. Part 1—dose escalation:
      DLBCL (de novo or histologically transformed).
      HGBCL.
      PMBCL.
      FL.
      MZL (nodal, extranodal of mucosa-associated lymphoid tissue, or splenic).
      SLL.
   b. Part 2—dose expansion:
      DLBCL, NOS (according to the WHO 2016 classification) including histologically transformed from indolent lymphomas except chronic lymphocytic leukemia (CLL) and Waldenstrom macroglobulinemia (WM).
      Subjects with “double-hit” or “triple-hit” DLBCL (technically classified in WHO 2016 as HGBCL, with MYC and BCL2 and/or BCL6 translocations) are included.
      Note: other double-/triple-hit lymphomas are not eligible.
      FL grade 1-3A at initial diagnosis with no evidence of clinical or histological transformation.
5. Relapsed or refractory disease and previously treated with at least 2 lines of systemic antineoplastic therapy including at least 1 anti-CD20 mAb-containing therapy. Note: Relapsed disease is defined as disease that has recurred >6 months after completion of therapy. Refractory disease is defined as disease that either progressed during therapy or progressed within 6 months (<6 months) of completion of therapy.
6. For DLBCL subjects not eligible for HDT with ASCT, the decision must have been based on age, ECOG performance status, comorbidities, and/or insufficient response to prior treatment.
7. Subjects with indolent lymphoma (FL grade 1-3A, MZL, SLL) must have active disease with a need for treatment initiation based on symptoms and/or disease burden.
8. CD20 positivity at representative (previous or current) tumor biopsy based on the pathology report.
9. Measurable disease:
   a. FDG-avid lymphomas: measurable disease with CT (or MRI) scan with involvement of 2 or more clearly demarcated lesions/nodes with a long axis >1.5 cm and short axis >1.0 cm (or 1 clearly demarcated lesion/node with a long axis >2.0 cm and short axis ≥1.0 cm) and FDG PET scan that demonstrates positive lesion(s) compatible with CT (or MRI) defined anatomical tumor sites.
b. FDG-nonavid lymphomas: Measurable disease with CT (or MRI) scan with involvement of 2 or more clearly demarcated lesions/nodes with a long axis >1.5 cm and short axis >1.0 cm or 1 clearly demarcated lesion/node with a long axis >2.0 cm and short axis ≥1.0 cm.
10. Criterion modified per Amendment 2. 10.1 ECOG PS score ≤2.
11. Lymphocyte counts <5×109/L.
12. Platelet counts ≥50×109/L.
13. Absolute neutrophil counts ≥1.0×109/L; growth factor support allowed in case of bone marrow involvement.
14. Subject must meet the following criteria regarding time since previous antineoplastic agent(s):
   a. At least 4 weeks from last dose of non-investigational systemic chemotherapy.
   b. At least 4 weeks or 5 half-lives from last dose of other non-investigational antineoplastic agents, whichever is shorter (except any anti-CD20 mAb).
   c. At least 5 half-lives from last dose of investigational agents except for prior (CAR-T) therapy from which 100 days must pass prior to first GEN3013 administration.
15. Resolution of toxicities from prior therapy to a grade that does not contraindicate trial participation in the opinion of the investigator.
16. Before the first dose of epcoritamab, during the trial, and for 12 months after last administration of epcoritamab, a woman must either be not of reproductive potential or of reproductive potential and practicing a birth control, as defined by the Investigator and consistent with local regulations.
17. A man who is sexually active with a woman of reproductive potential must agree to practice birth control, as defined by the Investigator and consistent with local regulations, during the trial and for 12 months after receiving the last dose of epcoritamab.
18. A female subject is eligible to participate if she is not pregnant, not breastfeeding, discontinues breastfeeding prior to first dose (and does not breastfeed for the duration of the trial or at least 12 months after the last trial treatment administration) and not expecting to conceive children within the projected duration of the trial and for at least 12 months after the last trial treatment administration and one of the following conditions applies:
   a. Not a woman of reproductive potential.
   b. A woman of reproductive potential and agrees to use at least one adequate form of contraception during and for 12 months after the last dose of epcoritamab.
20. Women must agree not to donate eggs (ova, oocytes) for the purposes of assisted reproduction during the trial and for 12 months after receiving the last dose of epcoritamab. Men must also not donate sperm during the trial and for 12 months after receiving the last dose of epcoritamab.
21. Subject must be willing and able to adhere to the prohibitions and restrictions specified in this protocol.
22. Life expectancy >3 months on SOC treatment.

### Exclusion Criteria—Dose Escalation and Expansion

Any potential subject who meets any of the following criteria will be excluded from participating in the trial.
1. Primary CNS lymphoma or CNS involvement by lymphoma on brain MRI/CT at screening.
2. Subjects not eligible for HDT-ASCT due to personal choice, social issues, or similar.
3. Known past or current malignancy other than inclusion diagnosis, except for:
   a. Cervical carcinoma of Stage 1B or less.
   b. Non-invasive basal cell or squamous cell skin carcinoma.
   c. Non-invasive, superficial bladder cancer.
   d. Prostate cancer with a current prostate-stimulating antigen level <0.1 ng/ml.
   e. Any curable cancer with a complete response (CR) of >2 years duration.
4. History of confirmed progressive multifocal leukoencephalopathy (PML).
5. AST and/or ALT >3 times the upper limit of normal (xULN).
6. Total bilirubin >1.5×ULN (unless due to Gilbert syndrome).
7. Estimated GFR <45 mL/min/1.73 m2 (Appendix 1).
8. Known clinically significant cardiac disease, including:
   a. Onset of unstable angina pectoris within 6 months of signing ICF.
   b. Acute myocardial infarction, intracranial bleed, or stroke within 6 months of signing ICF. c. Congestive heart failure (grade III or IV as classified by the NYHA and/or known decrease ejection fraction of <45%).
   d. Screening 12-lead ECG showing a baseline QT interval as corrected by Fridericia's formula (QTcF)>470 msec.
9. Acute bacterial, viral, or fungal infection at baseline, confirmed by a positive blood culture within 72 hours before first epcoritamab administration or by clinical judgment in the absence of a positive blood culture.
10. Confirmed history or current autoimmune disease or other diseases resulting in permanent immunosuppression or requiring permanent immunosuppressive therapy (except low-dose corticosteroid for rheumatoid arthritis).
11. Seizure disorder requiring therapy (such as steroids or anti-epileptics).
12. Prior ASCT within 100 days before first epcoritamab administration.
13. Prior allogeneic stem cell transplantation.
14. Prior solid organ transplantation.
15. Prior therapy with an investigational bispecific antibody targeting CD3 and CD20.
16. Prior treatment with CAR-T cell therapy within 100 days before first epcoritamab administration.
17. Chronic ongoing infectious diseases requiring treatment (excluding prophylactic treatment) at the time of enrolment or within the previous 2 weeks prior to the first dose of epcoritamab.
18. Active hepatitis B (HepB DNA positive) or hepatitis C (HepC RNA positive). If laboratory evidence for a chronic infection with hepatitis B, close monitoring and prophylactic therapy is required.
19. Known history of seropositivity for HIV infection.
20. Prior exposure to live or live attenuated vaccine within 4 weeks before the first dose of epcoritamab or anticipation that such a live attenuated vaccine will be required during the study. Note: Seasonal influenza vaccines are generally killed virus vaccines and are permitted however, intranasal influenza vaccines are live attenuated and are not allowed.
21. Contraindication to all uric acid lowering agents.
22. Any condition for which, in the opinion of the investigator, participation would not be in the best interest of the subject (e.g., compromise the well-being) or that could prevent, limit, or confound the protocol-specified assessments.
23. Subject has known allergies, hypersensitivity, or intolerance to epcoritamab or its excipients.
24. History of severe allergic or anaphylactic reactions to monoclonal antibody therapy.
25. Subject has taken any disallowed therapies, Concomitant Medications and Therapies before the planned first dose of epcoritamab.
26. Subject has received an investigational non-antineoplastic drug (including investigational vaccines) or used an invasive investigational medical device within 4 weeks (or 5 half-lives, whichever is shorter) before the planned first dose of epcoritamab (or is currently enrolled in an investigational trial).
27. Positive test results for HTLV-1 (only for expansion).
28. Receiving immunosuppressive therapy, including more than the equivalent of 20 mg of prednisone* daily, unless for underlying disease control. * Not approved in Japan.
29. Receiving immunostimulatory agent.

### Summary of Clinical Data as of Jan. 31, 2022

This summarizes data for GCT3013-01 (safety for the dose-escalation and expansion parts, efficacy for the dose escalation part, and PK for the dose escalation part) and safety data for GCT3013-04 dose escalation part. No DLT has occurred, the MTD has not been reached, and the RP2D was declared to be a full dose of 48 mg. All 68 subjects in the GCT3013-01 dose escalation part experienced at least 1 TEAE; 80.9% of subjects had at least one grade 3 or higher TEAE. The most common TEAEs were pyrexia, CRS, injection site reaction, and fatigue. There were 13 subjects with TEAEs leading to death (11 subjects with malignant neoplasm progression, 1 with euthanasia [also in the context of progressive disease], and 1 with COVID-19 pneumonia). SAEs were reported for 67.6% of subjects. The most common SAE considered related to trial drug was pyrexia, which was reported as a symptom of CRS. TEAEs leading to permanent treatment discontinuation were reported in 13.2% of subjects. AESIs included the following: 40 subjects experienced CRS, 4 subjects experienced neurological symptoms considered by the investigator to be immune mediated, and 1 subject experienced clinical tumor lysis syndrome.

A total of 286 subjects (98.3%) in the GCT3013-01 expansion part experienced at least 1 TEAE; 59.8% of subjects had at least one grade 3 or higher TEAE. The most common TEAEs were CRS, fatigue, and pyrexia. There were 15 subjects with TEAEs leading to death (2 subjects with COVID-19, 1 subject each with necrotizing fasciitis, pneumonia, aspiration pneumonia, progressive multifocal leukencephalopathy, septic shock, fatigue, general physical health deterioration, cerebral hemorrhage, immune effector cell-associated neurotoxicity syndrome, lung opacity, pulmonary embolism, myocardial infraction, hepatotoxicity). SAEs were reported for 60.5% of subjects. The most common SAE considered related to trial drug was CRS. TEAEs leading to permanent treatment discontinuation were reported in 5.8% of subjects. AESIs included the following: 163 subjects experienced CRS, 21 subjects experienced ICANS, and 4 subject experienced clinical tumor lysis syndrome.

All 7 subjects in the GCT3013-04 dose escalation part experienced at least 1 TEAE; 71.4% of subjects had at least one grade 3 or higher TEAE. The most common TEAEs were CRS and injection site erythema. No subjects experienced a TEAE leading to treatment discontinuation, an SAE, or a TEAE leading to death. Two subjects experienced TEAEs leading to dose modification. AESIs of CRS occurred in 6 subjects (85.7%).

Epcoritamab administration induced rapid and sustained depletion of circulating B-cells (in the subset of subjects with detectable B-cells, which are absent in most patients due to prior anti-CD20 therapy) and increases in peripheral T-cells and circulating IFNγ. No significant ADA (titers ≥1) against epcoritamab have been observed in patients. In the GCT3013-01 dose escalation part, for all dose levels combined, the ORR was 44.1%; for the RP2D level (48 mg), the ORR was 66.7%.

### Class Effects

Adverse reactions reported for the class of compounds that epcoritamab belongs to, i.e., bispecific T-cell engagers, include CRS, neurological symptoms, and infection.

B-cell depletion may lead to increased risk for infections. Infections should be managed according to best medical practice. Monitoring of latent viral infection, e.g, hepatitis B or cytomegalovirus, for subjects at risk should be performed during and after eporitamab treatment. A total of 181 subjects in the GCT3013-01 trial and GCT3013-04 trial escalation part experienced infections.

CRS has been reported with epcoritamab, and other compounds or drugs targeting CD3 and chimeric antigen receptor T-cells. Close monitoring of vital signs, in particular temperature, blood pressure, and oxygen saturation, as well as laboratory assessments of hematology, liver and kidney parameters is important to secure a timely start of supportive care as needed. Supportive care based on (Lee et al., 2019) and (Neelapu et al., 2017) can include, but is not limited to:
Infusion of saline
Systemic glucocorticosteroids, antihistamines, antipyrexia
Support for blood pressure
Support for ventilation
Monoclonal antibody against IL-6R, IL-6, or IL-1 (e.g, tocilizumab, siltuximab, and/or anakinra)

Additional CRS risk reducing measures were implemented including prophylactic corticosteroid administration for 4 consecutive days in relation to epcoritamab administration in Cycle 1. The majority of CRS events have been reported as grade 1 and 2; only 9 subjects reported grade 3 CRS and only 1 subject (MCL cohort) reported grade 4 CRS.

In the GCT3013-01 study, 203 subjects reported at least 1 CRS event. The majority of cases were of grade 1 or grade 2 CRS, whereas grade 3 CRS occurred in only 9 patients and grade 4 in only 1 patient, which indicates grade 1 or grade 2 CRS is a very common event, whereas grade 3 appears to be common, and grade 4 uncommon. Of those, 40 (58.8%) subjects (20 subjects-grade 1; 20 subjects-grade 2) were in the GCT3013-01 dose escalation part, 163 (56.0%) subjects (95 subjects-grade 1; 58 subjects-grade 2; 9 subjects-grade 3; 1 subject-grade 4) in the GCT3013-01 expansion part. All cases of CRS were assessed as related to epcoritamab and recovered/resolved.

Neurological symptoms ranging from confusion to fatal cerebral edema have been reported with other compounds or drugs targeting CD3. Neurological assessment should be performed according to ICANS evaluation (Lee et al., 2019). The grading of ICANS requires assessment of the 10-point immune effector cell-associated encephalopathy score as well as evaluation of 4 other neurological domains: level of consciousness, seizures, motor symptoms, and signs of raised intracranial pressure/cerebral edema, which may occur with or without encephalopathy (refer to the protocol). Close monitoring of mental status during treatment is important to secure a timely start of supportive care as needed. Supportive care can include, but is not limited to:
Initiation of IV hydration.
Withhold oral intake
Avoid medications that cause central nervous system depression
Initiation of corticosteroids
Anti-cytokine therapy
Anti-convulsive therapy

A total of 25 subjects experienced adverse events related to neurological symptoms (AESIs). Twenty-one subjects in the GCT3013-01 expansion part developed ICANS, which was fatal in 1 subject. Four of the 25 subjects were from the GCT3013-01 dose escalation part and experienced 3 serious AESIs of grade 3 depressed level of consciousness, grade 3 hypersomnia, grade 1 partial seizure, and 2 non-serious AESIs of grade 1 dysgraphia and grade 1 agraphia.

### Efficacy

The objective response rate (ORR) (CR+PR) based on IRC assessment was 55.6% (95% CI: 38.1%, 72.1%) with 44.4% CR and 11.1% PR.

By IRC, median duration of response was not reached. The estimated percentage of subjects remaining in response at 6 months was 69.3% (all responders) and at 9 months was 59.4% (all responders), for subjects achieving CR the estimated percentage of subjects remaining in response at 6 and 9 months was 61.9%.

The median progression free survival was 4.1 months (95% CI: 1.2, NE).

Median overall survival was NR. The estimated percentage of subjects alive at 9 months was 59.8% (95% CI: 38.5%, 75.8%).

### Nov. 18, 2022 Data Cut.

As of Nov. 18, 2022, of 157 patients (median age, 64 y; 20-83) with LBCL (including DLBCL [n=139], HGBCL [n=9], PMBCL [n=4], and FL grade 3B [n=5]), 36 remained on treatment. Patients had a median of 1.6 year from initial diagnosis to first dose and a median of 3 (2-11) prior treatment lines; 61% of the patients had primary refractory disease, and 39% had prior CAR T, of whom 75% progressed within 6 months of treatment. Patients received a median of 5 treatments Cs. Median follow-up was 20 months (0.3+ to 28.2). Overall response and CR rates were 63.1% and 39.5%, respectively, and were consistent for DLBCL (61.9% and 39.6%). The mean duration of CR was 20.8 months (95% CI, 15.8—NR). Median time to CR was 2.7 months; 8 patients converted from partial response to CR at ≥36 wk. Median Overall Survival (mOS) was 18.5 months (95% CI, 11.7—NR) with an estimated 58% of patients alive at 12 mo. Median OS was not reached (NR) in patients who achieved CR. Additional outcomes for patients with CR are shown in Table 13. Most common treatment emergent adverse events (TEAEs) of any grade (G) were CRS (51%), neutropenia (24%), pyrexia (24%), fatigue (23%), nausea (22%), and diarrhea (21%). Nine patients (6%) had G1-2 ICANS, and 1 patient had a G5 event with confounding factors. Fatal TEAEs were reported in 15 patients; 2 were considered related (COVID-19 and ICANS). CRS was predominantly low grade (G1-2: 48%; G3: 3%) and occurred following the first full dose (C1D15). One patient discontinued treatment due to CRS (G1). No new safety signals were observed.

Data on Best Overall Response based on IRC Assessment, Lugano Criteria, Duration of Complete Response based on Investigator Assessment, Lugano Criteria, Progression-Free Survival based on IRC Assessment, Lugano Criteria, Primary Definition, Overall Survival from Full Analysis Set are shown in Tables 14, 15, 16 and 17, respectively. Event-Level CRS Summary data and Summary of AESI are shown in Tables 18 and 19, respectively.

Conclusion: These long-term data reaffirm single agent subcutaneous (SC) epcoritamab induces impressive and durable CRs and a manageable safety profile in hard-to-treat Resistant/Relapsed DLBCL/LBCL patients. These results are highly encouraging and support the additional exploration of epcoritamab in ongoing phase 3 studies, across lines of therapy and in various combinations.

TABLE 13
Outcomes for patients who achieved a CR (n = 62)

| | DOR* in CR | | PFS* at | | OS* at |
|---|---|---|---|---|---|
| mDoCR | pts at 9, 12, | mPFS | 9, 12, | mOS | 9, 12, |
| mo | 15 mo % | mo | 15 mo % | mo | 15 mo % |
| 20.8 | 88.3, | NR | 91.1, | NR | 98.3, |
| 95% CI, | 77.3, | 95% CI, | 87.2, | 95% CI, | 95.0, |
| 15.8-NR | 67.9 | 18.5-NR | 81.3 | NR-NR | 88.3 |

*Kaplan-Meier estimate

TABLE 14
Best Overall Response based on IRC Assessment, Lugano Criteria - GCT3013-01
Expansion Part, subjects in aNHL Cohort - Full Analysis Set

| | aNHL Cohort | | | | | |
|---|---|---|---|---|---|---|
| | DLBCL | | Other | | LBCL | |
| Best Overall Response | (N = 139) | | (N = 18) | | (N = 157) | |
| Complete Response (CR) | 55 | (39.6%) | 7 | (38.9%) | 62 | (39.5%) |
| Partial Response (PR) | 31 | (22.3%) | 6 | (33.3%) | 37 | (23.6%) |
| Stable Disease (SD) | 4 | (2.9%) | 1 | (5.6%) | 5 | (3.2%) |
| Progressive Disease (PD) | 33 | (23.7%) | 4 | (22.2%) | 37 | (23.6%) |
| Not Evaluable (NE) | 16 | (11.5%) | 0 | | 16 | (10.2%) |
| Overall Response Rate (ORR) [a] | 86 | (61.9%) | 13 | (72.2%) | 99 | (63.1%) |
| (95% CI) [b] | (53.3%, 70.0%) | | (46.5%, 90.3%) | | (55.0%, 70.6%) | |
| Complete Response Rate (CRR) | 55 | (39.6%) | 7 | (38.9%) | 62 | (39.5%) |
| (95% CI) [b] | (31.4%, 48.2%) | | (17.3%, 64.3%) | | (31.8%, 47.6%) | |
| Disease Control Rate (DCR) [c] | 90 | (64.7%) | 14 | (77.8%) | 104 | (66.2%) |

| | | | |
|---|---|---|---|
| (95% CI) [b] | (56.2%, 72.7%) | (52.4%, 93.6%) | (58.3%, 73.6%) |

Note:
Percentages calculated based on number of subjects in FAS.
[a] CR + PR
[b] based on the Clopper and Pearson method
[c] CR + PR + SD
Tigermed\GEN3013-01\t_asco_9.sas\T_asco_9
Snapshot date: 8 DEC. 2022; Data Cutoff: 18 NOV. 2022; Date Generated: 29 JAN. 2023 03:52

| | aNHL Cohort | | |
|---|---|---|---|
| | DLBCL | Other | LBCL |
| | (N = 139) | (N = 18) | (N = 157) |
| Number of Responders | 86 | 13 | 99 |
| Number of Events | 43 (50%) | 5 (38.5%) | 48 (48.5%) |
| Number of Censored | 43 (50%) | 8 (61.5%) | 51 (51.5%) |
| Reason for censoring | | | |
| Clinical cutoff | 34 (79.1%) | 4 (50.0%) | 38 (74.5%) |
| New anti-lymphoma therapy | 9 (20.9%) | 3 (37.5%) | 12 (23.5%) |
| Lost to follow-up | 0 | 0 | 0 |
| Subject withdrew consent | 0 | 1 (12.5%) | 1 (2.0%) |
| DOR (months) | | | |
| Min, Max | 0.0+, 22.7+ | 0.0+, 15.4+ | 0.0+, 22.7+ |
| Median follow-up (95% CI)[a] | 15.6 (15.2, 18.0) | 9.5 (3.1, 14.5) | 15.4 (15.1, 16.1) |
| 25% quartile (95% CI)[b] | 3.1 (1.6, 6.6) | 1.8 (1.2, NR) | 2.9 (1.7, 5.6) |
| Median (95% CI)[b] | 15.5 (9.7, 20.8) | NR (1.5, NR) | 15.5 (9.7, 20.8) |
| 75% quartile (95% CI)[b] | NR (20.8, NR) | NR (NR, NR) | NR (20.8, NR) |

Note:
Symbol ‘+’ indicated a censored value.
NR = Not reached.
[a]based on reverse Kaplan-Meier estimate,
[b]based on Kaplan-Meier estimate.
Tigermed\GEN3013-01\t_asco_10.sas\t_asco_10
Snapshot date: 8 DEC. 2022; Data Cutoff: 18 NOV. 2022; Date Generated: 29 JAN. 2023 03:49

TABLE 15
Duration of Complete Response based on Investigator Assessment, Lugano Criteria -
GCT3013-01 Expansion Part, subjects in aNHL Cohort - Full Analysis Set

| | aNHL Cohort | | |
|---|---|---|---|
| | DLBCL | Other | LBCL |
| | (N = 139) | (N = 18) | (N = 157) |
| Number of Responders | 57 | 7 | 64 |
| Number of Events | 17 (29.8%) | 1 (14.3%) | 18 (28.1%) |
| Number of Censored | 40 (70.2%) | 6 (85.7%) | 46 (71.9%) |
| Reason for censoring | | | |
| Clinical cutoff | 35 (87.5%) | 4 (66.7% | 39 (84.8%) |
| New anti-lymphoma therapy | 5 (12.5%) | 2 (33.3%) | 7 (15.2%) |
| Lost to follow-up | 0 | 0 | 0 |
| Subject withdrew consent | 0 | 0 | 0 |
| DoCR (months) | | | |
| Min, Max | 0.0+, 21.8+ | 0.5+, 15.9+ | 0.0+, 21.8+ |
| Median follow-up (95% CI)[a] | 15.2 (14.6, 15.6) | 8.3 (0.5, NR) | 15.1 (14.0, 15.4) |
| 25% quartile (95% CI)[b] | 13.1 (9.7, 19.3) | NR (1.4, NR) | 13.1 (9.7, 19.3) |
| Median (95% CI)[b] | NR (15.8, NR) | NR (1.4, NR) | NR (15.8, NR) |
| 75% quartile (95% CI)[b] | NR (NR, NR) | NR (NR, NR) | NR (NR, NR) |
| Estimate percentage of patients remaining | | | |
| in complete response (95% CI)[b] | | | |
| 3-month | 94.3% (83.2%, 98.1%) | 83.3% (27.3%, 97.5%) | 93.2% (82.8%, 97.4%) |
| 6-month | 90.3% (78.3%, 95.9%) | 83.3% (27.3%, 97.5%) | 89.6% (78.2%, 95.2%) |
| 9-month | 88.4% (75.9%, 94.6%) | 83.3% (27.3%, 97.5%) | 87.7% (76.0%, 94.0%) |
| 12-month | 79.3% (64.9%, 88.4%) | 83.3% (27.3%, 97.5%) | 79.2% (65.4%, 88.0%) |
| 15-month | 69.3% (53.5%, 80.7%) | 83.3% (27.3%, 97.5%) | 69.5% (54.2%, 80.6%) |
| 18-month | 64.4% (46.5%, 77.7%) | NR (NR, NR) | 64.9% (47.7%, 77.7%) |

Note:
Symbol ‘+’ indicated a censored value.
NR = Not reached.
[a]based on reverse Kaplan-Meier estimate.
[b]based on Kaplan-Meier estimate.
Tigermed\GEN3013-01\t_asco_12.sas\t_asco_11
Snapshot date: 8 DEC. 2022; Data Cutoff: 18 NOV. 2022; Date Generated: 29 JAN. 2023 03:49

TABLE 16
Progression-Free Survival based on IRC Assessment, Lugano Criteria, Primary Definition -
GCT3013-01 Expansion Part, subjects in aNHL Cohort - Full Analysis Set

| | aNHL Cohort | | |
|---|---|---|---|
| | DLBCL | Other | LBCL |
| | (N = 139) | (N = 18) | (N = 157) |
| Number of Events | 92 (66.2%) | 10 (55.6%) | 102 (65.0%) |
| Number of Censored | 47 (33.8%) | 8 (44.4%) | 55 (35.0%) |
| Reason for censoring | | | |
| Clinical cutoff | 34 (72.3%) | 4 (50.0%) | 38 (69.1%) |
| New anti-lymphoma therapy | 11 (23.4%) | 3 (37.5%) | 14 (25.5%) |
| Lost to follow-up | 0 | 0 | 0 |
| Subject withdrew consent | 0 | 1 (12.5%) | 1 (1.8%) |
| No baseline assessments | 0 | 0 | 0 |
| No post-baseline assessments | 2 (4.3%) | 0 | 2 (3.6%) |
| PFS (months) | | | |
| Min, Max | 0.0+, 23.6+ | 0.6, 17.1+ | 0.0+, 23.6+ |
| Median follow-up (95% CI)[a] | 17.1 (16.6, 19.2) | 10.9 (4.2, 16.8) | 16.9 (16.6, 17.5) |
| 25% quartile (95% CI)[b] | 1.4 (1.2, 1.8) | 2.8 (0.6, 3.0) | 1.4 (1.2, 2.5) |
| Median (95% CI)[b] | 4.4 (3.0, 8.8) | 3.8 (2.8, NR) | 4.4 (3.0, 8.8) |
| 75% quartile (95% CI)[b] | 20.5 (16.7, NR) | NR (4.6, NR) | 20.5 (16.7, NR) |
| Estimate percentage of patients remaining | | | |
| in progression free (95% CI)[b] | | | |
| 6-month | 45.7% (37.0%, 54.0%) | 43.8% (20.8%, 64.7%) | 45.4% (37.3%, 53.2%) |
| 9-month | 41.4% (32.8%, 49.8%) | 43.8% (20.8%, 64.7%) | 41.5% (33.4%, 49.3%) |
| 12-month | 39.7% (31.2%, 48.0%) | 43.8% (20.8%, 64.7%) | 39.8% (31.8%, 47.7%) |

Note:
Symbol ‘+’ indicated a censored value.
NR = Not reached.
[a]based on reverse Kaplan-Meier estimate.
[b]based on Kaplan-Meier estimate.
Tigermed\GEN3013-01\t_asco_12.sas\t_asco_12
Snapshot date: 8 DEC. 2022; Data Cutoff: 18 NOV. 2022; Date Generated: 29 JAN. 2023 03:49

TABLE 17
Overall Survival - GCT3013-01 Expansion Part, subjects in aNHL Cohort - Full Analysis Set

| | aNHL Cohort | | |
|---|---|---|---|
| | DLBCL | Other | LBCL |
| | (N = 139) | (N = 18) | (N = 157) |
| Number of Events | 70 (50.4%) | 10 (55.6%) | 80 (51.0%) |
| Number of Censored | 69 (49.6%) | 8 (44.4%) | 77 (49.0%) |
| OS (months) | | | |
| Min, Max | 0.3, 28.2+ | 0.7, 20.5+ | 0.3, 28.2+ |
| Median follow-up (95% CI)[a] | 20.0 (19.4, 21.0) | 18.2 (15.4, 20.1) | 20.0 (19.2, 20.5) |
| 25% quartile (95% CI)[b] | 4.5 (2.9, 6.7) | 4.3 (0.7, 9.3) | 4.3 (2.9, 6.1) |
| Median (95% CI)[b] | 19.4 (11.7, NR) | 13.2 (4.3, NR) | 18.5 (11.7, NR) |
| 75% quartile (95% CI)[b] | NR (NR, NR) | NR (13.2, NR) | NR (NR, NR) |
| Estimate percentage of patients | | | |
| remaining alive (95% CI)[b] | | | |
| 6-month | 70.6% (62.2%, 77.5%) | 60.6% (34.6%, 79.0%) | 69.5% (61.5%, 76.1%) |
| 9-month | 63.0% (54.3%, 70.6%) | 60.6% (34.6%, 79.0%) | 62.7% (54.5%, 69.9%) |
| 12-month | 58.4% (49.6%, 66.2%) | 54.5% (29.2%, 74.2%) | 58.0% (49.7%, 65.4%) |
| 15-month | 52.9% (44.1%, 61.0%) | 42.4% (19.5%, 63.8%) | 51.7% (43.4%, 59.4%) |
| 18-month | 51.3% (42.5%, 59.5%) | 42.4% (19.5%, 63.8%) | 50.3% (42.0%, 58.0%) |

Tigermed\GEN3013-01\t_asco_13.sas\t_asco_13
Snapshot date: 8 DEC. 2022; Data Cutoff: 18 NOV. 2022; Data Generated: 29 JAN. 2023 03:49

TABLE 18
Event-Level CRS Summary - GCT3013-01 Expansion Part,
subjects in aNHL Cohort - Full Analysis Set

| | aNHL Cohort | | | | | |
|---|---|---|---|---|---|---|
| | DLBCL | | Other | | LBCL | |
| | (N = 139) | | (N = 18) | | (N = 157) | |
| Subjects with at least one CRS event | 69 | | 11 | | 80 | |
| Number of episodes per subject [a] | | | | | | |
| 1 event | 47 | (68.1%) | 6 | (54.5%) | 53 | (66.3%) |
| 2 events | 16 | (23.2%) | 5 | (45.5%) | 21 | (26.3%) |
| 3 events | 4 | (5.8%) | 0 | | 4 | (5.0%) |
| 4 events | 1 | (1.4%) | 0 | | 1 | (1.3%) |
| 5 events | 1 | (1.4%) | 0 | | 1 | (1.3%) |
| Number of CRS events | 100 | | 16 | | 116 | |
| Grade 1 | 67 | (67.0%) | 12 | (75.0%) | 79 | (68.1%) |
| Grade 2 | 28 | (28.0%) | 4 | (25.0%) | 32 | (27.6%) |
| Grade 3 | 5 | (5.0%) | 0 | | 5 | (4.3%) |
| Grade 4 | 0 | | 0 | | 0 | |
| Grade 5 | 0 | | 0 | | 0 | |
| Occurrence of any CRS Signs and Symptoms | 100 | (100%) | 16 | (100%) | 116 | (100%) |
| Fever | 98 | (98.0%) | 16 | (100%) | 114 | (98.3%) |
| Hypotension | 24 | (24.0%) | 3 | (18.8%) | 27 | (23.3%) |
| Hypoxia | 17 | (17.0%) | 1 | (6.3%) | 18 | (15.5%) |
| Other | 17 | (17.0%) | 1 | (6.3%) | 18 | (15.5%) |
| CRS event | | | | | | |
| Treated with anti-cytokine therapy | 25 | (25.0%) | 3 | (18.8%) | 28 | (24.1%) |
| Tocilizumab | 25 | (25.0%) | 3 | (18.8%) | 28 | (24.1%) |
| Number of tocilizumab doses taken | | | | | | |
| 1 | 23 | (23.0%) | 3 | (18.8%) | 26 | (22.4%) |
| 2 | 1 | (1.0%) | 0 | | 1 | (0.9%) |
| 3 | 0 | | 0 | | 0 | |
| >=4 | 1 | (1.0%) | 0 | | 1 | (0.9%) |
| Other anti-cytokine | 0 | | 0 | | 0 | |
| Treated with corticosteroid for CRS | 15 | (15.0%) | 5 | (31.3%) | 20 | (17.2%) |
| Leading to dose delay/interruption | 8 | (8.0%) | 5 | (31.3%) | 13 | (11.2%) |
| Leading to treatment discontinuation | 1 | (1.0%) | 0 | | 1 | (0.9%) |
| Time to CRS onset from most recent dosing (days) | | | | | | |
| n | 100 | | 16 | | 116 | |
| Mean (Std Dev) | 3.0 | (1.93) | 3.8 | (1.91) | 3.1 | (1.93) |
| Median | 2.0 | | 3.5 | | 2.0 | |
| Min, Max | 1, 11 | | 1, 6 | | 1, 11 | |
| Time to CRS resolution (days) | | | | | | |
| Resolved CRS | 98 | (98.0%) | 16 | (100%) | 114 | (98.3%) |
| Mean (STD Dev) | 2.8 | (2.21) | 4.1 | (6.80) | 3.0 | (3.24) |

| | | | |
|---|---|---|---|
| Median | 2.0 | 1.0 | 2.0 |
| Min, Max | 1, 15 | 1, 27 | 1, 27 |

Note:
CRS events are graded according to Lee et al 2019.
[a] Percentage calculated based on number of subjects with at least one CRS event.
Other percentages are calculated based on number of CRS events in the analysis group.
Tigermed\GEN3013-01\t_asco_26.sas\t_asco_26
Snapshot date: 8 DEC. 2022; Data Cutoff: 18 NOV. 2022; Date Generated: 29 JAN. 2023 03:51

TABLE 19
Summary of AESI: Immune Effector Cell-Associated Neurotoxicity Syndrome (ICANS) -
GCT3013-01 Expansion Part, subjects in aNHL Cohort - Full Analysis Set

| | aNHL Cohort | | |
|---|---|---|---|
| | DLBCL | Other | LBCL |
| | (N = 139) | (N = 18) | (N = 157) |
| Subjects with at least one ICANS event | 9 (6.5%) | 1 (5.6%) | 10 (6.4%) |
| Grade 1 | 6 (4.3%) | 1 (5.6%) | 7 (4.5%) |
| Grade 2 | 2 (1.4%) | 0 | 2 (1.3%) |
| Grade 3 | 0 | 0 | 0 |
| Grade 4 | 0 | 0 | 0 |
| Grade 5 | 1 (0.7%) | 0 | 1 (0.6%) |
| Worst On-treatment ICE score | | | |
| 10 | 2 (1.4%) | 1 (5.6%) | 3 (1.9%) |
| 7-9 | 5 (3.6%) | 0 | 5 (3.2%) |
| 3-6 | 1 (0.7%) | 0 | 1 (0.6%) |
| 0-2 | 0 | 0 | 0 |
| Missing | 1 (0.7%) | 0 | 1 (0.6%) |
| Subject with ICANS event | | | |
| Leading to dose delay/interruption | 3 (2.2%) | 0 | 3 (1.9%) |
| Leading to treatment discontinuation | 1 (0.7%) | 0 | 1 (0.6%) |

Note:
ICANS events are graded according to Lee et al 2019.
[a] Percentage calculated based on subjects with at least 1 ICANS event.
[b] Based on longest recorded CRS duration in subjects with >1 ICANS event.

1. A method of treating a B-cell Non-Hodgkin Lymphoma (B-NHL) in a human subject, the method comprising administering subcutaneously to a human subject in need thereof, a bispecific antibody at a dose of at least 24 mg, said bispecific antibody being a full-length antibody, that comprises
i. a first binding arm comprising a first antigen-binding region binding to human CD3s (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to provide a response in said subject, the response having a duration of 6 months or longer, and wherein the response comprises at least a partial response (PR).

2-4. (canceled)

5. The method in accordance with claim 1, wherein said response comprises a complete response, and the response has a duration of 8 months or longer.

6-8. (canceled)

9. A method of achieving negative minimal residual disease (MRD) status/MRD negativity in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
i. a first binding arm comprising a first antigen-binding region binding to human CD3s (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to said subject for a time and/or a number of treatment cycles sufficient to provide a negative minimal residual disease (MRD) status/MRD negativity.

10. (canceled)

11. A method of decreasing a risk of relapse and/or disease progression in a human subject having B-cell Non-Hodgkin Lymphoma (B-NHL), the method comprising administering subcutaneously to said subject, a bispecific antibody at a dose of at least 24 mg, said bispecific antibody being a full-length antibody, that comprises
i. a first binding arm comprising a first antigen-binding region binding to human CD3s (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein said bispecific antibody is administered to provide a negative minimal residual disease (MRD) status/MRD negativity, wherein the negative MRD status/MRD negativity is indicative of a decreased risk of relapse and/or disease progression.

12. The method in accordance with claim 1, wherein the said B-NHL is selected from a large B-cell lymphoma (LBCL), diffuse large B-cell lymphoma (DLBCL), high-grade B-cell lymphoma (HGBCL), primary mediastinal large B-cell lymphoma (PMBCL), mantle cell lymphoma, follicular lymphoma (FL), marginal-zone lymphoma, small lymphocytic lymphoma or relapsed or refractory large B-cell lymphoma.

13-19. (canceled)

20. The method in accordance with claim 1, wherein said human subject has relapsed or refractory large B-cell lymphoma and has received 1, 2, 3, 4, 5 or 6 prior lines of treatment of said B-NHL.

21. The method in accordance with claim 20, wherein said subject has received a prior line of treatment which is
(a) chimeric antigen receptor T (CAR-T) cell therapy; or
(b) a CD20 monospecific antibody therapy.

22-28. (canceled)

29. A method of treating relapsed/refractory large B-cell lymphoma in a human subject, the method comprising administering to said subject, a bispecific antibody at a dose of at least 24 mg, said bispecific antibody being a full-length antibody, that comprises
i. a first binding arm comprising a first antigen-binding region binding to human CD3s (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein the subject achieves a complete response and a minimal residual disease (MRD) negativity when the antibody has been administered for 6 months or more.

30-32. (canceled)

33. The method in accordance with claim 29, wherein the subject (a) is CAR T-naïve ; (b) has primary refractory disease; (c) is refractory to prior Car T treatment; and/or (d) has DH/TH rearrangements.

34-38. (canceled)

39. The method in accordance with claim 1, wherein said bispecific antibody is administered (a) at a dose in the range of between 40 mg to 100 mg; (b) at a dose of at least 48 mg; (c) at a dose of at least 60 mg; (d) at a dose of 48 mg; or (e) at a dose of 60 mg.

40-43. (canceled)

44. The method in accordance with claim 1, wherein said dose is administered weekly at least 4 times.

45. (canceled)

46. The method in accordance with claim 44, wherein after said weekly administration, said antibody is administered once every two weeks at least 6 times.

47. (canceled)

48. The method in accordance with claim 46, wherein after said administration once every two weeks, said antibody is administered once every four weeks.

49. The method in accordance with claim 44, wherein prior to administering said weekly dose, a priming dose of said bispecific antibody is administered prior to administering the first dose of said weekly dose, wherein said priming dose is in the range of 50-1000 μg.

50-53. (canceled)

54. The method in accordance with claim 49, wherein after administering said priming dose and prior to administering said weekly dose, an intermediate dose of said bispecific antibody is administered, wherein said intermediate dose is in the range of 600-5000 μg.

55-59. (canceled)

60. The method in accordance with claim 54, wherein the method of treatment comprises administering the bispecific antibody subcutaneously in 28-day cycles, wherein on:
a) Day 1, 8, 15 and 22 of the first cycle, a priming dose is administered at day 1, an intermediate dose at day 8, and a dose of 48 mg at days 15, 22; for the first cycle; on
b) Day 1, 8, 15 and 22 of cycles 2-3, a dose of 48 mg is administered; on
c) Day 1, 15 of cycles 4-9, a dose of 48 mg is administered; and on
d) Day 1, of further subsequent cycles, a dose of 48 mg is administered.

61. The method in accordance with claim 54, wherein the method of treatment comprises administering the bispecific antibody subcutaneously in 28-day cycles, wherein on:
a) Day 1, 8, 15 and 22 the first cycle, a priming dose of 160 pg is administered at day 1, an intermediate dose of 800 pg at day 8, and a dose of 48 mg at days 15, 22; for the first cycle; on
b) Day 1, 8, 15 and 22 of cycles 2-3, a dose of 48 mg is administered; on
c) Day 1, 15 of cycles 4-9, a dose of 48 mg is administered; and on
d) Day 1, of further subsequent cycles, a dose of 48 mg is administered.

62. The method in accordance with claim 54-59, wherein the method of treatment comprises administering the bispecific antibody subcutaneously in 28-day cycles, wherein on:
a) Day 1, 8, 15 and 22 the first cycle, a priming dose is administered at day 1, an intermediate dose at day 8, and a dose of 60 mg at days 15, 22; for the first cycle; on
b) Day 1, 8, 15 and 22 of cycles 2-3, a dose of 60 mg is administered; on
c) Day 1, 15 of cycles 4-9, a dose of 60 mg is administered; and on
d) Day 1, of further subsequent cycles, a dose of 60 mg is administered.

63. The method in accordance with claim 54, wherein the method of treatment comprises administering the bispecific antibody subcutaneously in 28-day cycles, wherein on:
a) Day 1, 8, 15 and 22 the first cycle, a priming dose of 160 pg is administered at day 1, an intermediate dose of 800 pg at day 8, and a dose of 60 mg at days 15, 22; for the first cycle; on
b) Day 1, 8, 15 and 22 of cycles 2-3, a dose of 60 mg is administered;
c) Day 1, 15 of cycles 4-9, a dose of 60 mg is administered;
d) Day 1, of further subsequent cycles, a dose of 60 mg is administered.

64-66. (canceled)

67. The method in accordance with claim 1, wherein said subject is treated with prophylaxis for cytokine release syndrome (CRS), wherein said prophylaxis includes administration of a corticosteroid.

68-71. (canceled)

72. The method in accordance with claim 67, wherein said corticosteroid is prednisolone, administered at an intravenous dose of 100 mg, or equivalent thereof, including oral dose.

73. The method in accordance with claim 1, wherein said human subject is treated with premedication to reduce reactions to injections, wherein said premedication includes (a) antihistamines and/or (b) antipyretics.

74-82. (canceled)

83. The method in accordance with claim 73, wherein said premedication is administered during the first 28 day cycle, and prophylaxis is continued during a subsequent cycle, when in the last administration of the bispecific antibody of the previous cycle, the human subject experiences CRS greater than grade 1.

84-85. (canceled)

86. The method in accordance with claim 1, wherein the bispecific antibody comprises:
i. a first binding arm comprising a first antigen-binding region binding to human CD3s (epsilon), wherein said first antigen-binding region comprises a heavy chain variable (VH) region CDR1, CDR2, and CDR3 having the sequences as set forth in SEQ ID NOs: 1, 2, and 3, respectively, and a light chain variable (VL) region CDR1, CDR2, and CDR3 having the sequences as set forth in SEQ ID NO: 4, the sequence GTN, and the sequence as set forth in SEQ ID NO: 5, respectively, and
ii. a second binding arm comprising a second antigen-binding region binding to human CD20, wherein said second antigen-binding region comprises a heavy chain variable (VH) region CDR1, CDR2, and CDR3 having the sequences as set forth in SEQ ID NOs: 8, 9, and 10, respectively, and a light chain variable (VL) region CDR1, CDR2, and CDR3 having the sequences as set forth in SEQ ID NO: 11, the sequence DAS, and the sequence as set forth in SEQ ID NO: 12, respectively.

87. The method in accordance with claim 1, wherein the bispecific antibody comprises:
i. a first binding arm comprising a first antigen-binding region binding to human CD3s (epsilon), comprising a variable heavy chain region and a variable light chain region wherein the variable heavy chain region comprises SEQ ID NO: 6; and wherein the variable light chain region comprises SEQ ID NO: 7; and
ii. a second binding arm comprising a second antigen-binding region binding to human CD20, comprising a variable heavy chain region and a variable light chain region wherein the variable heavy chain region comprises SEQ ID NO: 13; and wherein the variable light chain region comprises SEQ ID NO: 14.

88-96. (canceled)

97. The method in accordance with claim 1, wherein the bispecific antibody comprises constant regions as defined in SEQ ID NOs: 19 and 20.

98. The method in accordance with claim 1, wherein the bispecific antibody comprises a heavy chain and a light chain as defined in SEQ ID NOs: 24 and 25, respectively, and a heavy chain and a light chain as defined in SEQ ID NOs: 26 and 27.

99. The method in accordance with claim 1, the bispecific antibody consists of a heavy chain and a light chain as defined in SEQ ID NOs: 24 and 25, respectively, and a heavy chain and a light chain as defined in SEQ ID NOs: 26 and 27.

100. The method in accordance with claim 1, wherein the bispecific antibody is epcoritamab, or a biosimilar thereof.

101. A method of predicting a likelihood of relapse and/or disease progression in a subject having B-cell Non-Hodgkin Lymphoma (B-NHL), the method comprising:
Measuring a minimal residual disease (MRD) status in the subject, wherein the subject receives or has received a bispecific antibody at a dose of at least 24 mg, said bispecific antibody being a full-length antibody, that comprises
i. a first binding arm comprising a first antigen-binding region binding to human CD3s (epsilon), comprising a variable heavy chain region and a variable light chain region wherein the variable heavy chain region comprises the CDR1, CDR2 and CDR3 sequences of SEQ ID NO: 6; and wherein the variable light chain region comprises the CDR1, CDR2 and CDR3 sequences of SEQ ID NO: 7; and
ii. a second binding arm comprising a second antigen-binding region binding to human CD20, comprising a variable heavy chain region and a variable light chain region wherein the variable heavy chain region comprises the CDR1, CDR2 and CDR3 sequences of SEQ ID NO: 13; and wherein the variable light chain region comprises the CDR1, CDR2 and CDR3 sequences of SEQ ID NO: 14;
wherein a positive MRD status is indicative of a likelihood of relapse and/or disease progression.

102-107. (canceled)


SEQUENCE LISTING (29 sequences)

SEQ ID NO: 1 - protein, 8 aa, synthetic construct
  REGION 1..8: note = CDR sequence
GFTFNTYA

SEQ ID NO: 2 - protein, 10 aa, synthetic construct
  REGION 1..10: note = CDR sequence
IRSKYNNYAT

SEQ ID NO: 3 - protein, 16 aa, synthetic construct
  REGION 1..16: note = CDR sequence
VRHGNFGNSYVSWFAY

SEQ ID NO: 4 - protein, 9 aa, synthetic construct
  REGION 1..9: note = CDR sequence
TGAVTTSNY

SEQ ID NO: 5 - protein, 9 aa, synthetic construct
  REGION 1..9: note = CDR sequence
ALWYSNLWV

SEQ ID NO: 6 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, synthetic construct
  REGION 1..8: note = CDR sequence
GFTFHDYA

SEQ ID NO: 9 - protein, 8 aa, synthetic construct
  REGION 1..8: note = CDR sequence
ISWNSGTI

SEQ ID NO: 10 - protein, 15 aa, synthetic construct
  REGION 1..15: note = CDR sequence
AKDIQYGNYYYGMDV

SEQ ID NO: 11 - protein, 6 aa, synthetic construct
  REGION 1..6: note = CDR sequence
QSVSSY

SEQ ID NO: 12 - protein, 9 aa, synthetic construct
  REGION 1..9: note = CDR sequence
QQRSNWPIT

SEQ ID NO: 13 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 18 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 19 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 20 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 25 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 27 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP